(19) United States
(12) Patent Application Publication
(10) Pub. No.: US 2024/0279320 A1
(43) Pub. Date: Aug. 22, 2024

(54) DOSAGE AND ADMINISTRATION OF ANTI-C5 ANTIBODIES FOR TREATING DERMATOMYOSITIS (DM)

(71) Applicant: Alexion Pharmaceuticals, Inc., Boston, MA (US)

(72) Inventors: Laura Marie Gault, Hingham, MA (US); Adrian Markus Kielhorn, Norwell, MA (US); Sanjay Nandkumar Rakhade, Winchester, MA (US); Usharbudh Shivraj Sohur, Milton, MA (US)

(73) Assignee: Alexion Pharmaceuticals, Inc., Boston, MA (US)

(21) Appl. No.: 18/568,011

(22) PCT Filed: Jun. 9, 2022
(86) PCT No.: PCT/US2022/032885

§ 371 Date: Dec. 7, 2023

Related U.S. Application Data
(60) Provisional application No. 63/210,280, filed on Jun. 14, 2021.

(51) Int. Cl. C07K 16/18; A61K 39/00; A61P 17/00; A61P 21/00; C07K 16/28

(52) U.S. Cl. CPC C07K 16/18; A61P 17/00; A61P 21/00; C07K 16/283; A61K 2039/505; A61K 2039/545

(57) ABSTRACT

Provided are dosages and methods for clinical treatment of dermatomyositis (DM), particularly severe and/or refractory DM, in human patients using an anti C5 antibody, or antigen binding fragment thereof (e.g., such as ravulizumab (ULTOMIRIS®)).

### CROSS REFERENCE TO RELATED APPLICATION

This application claims the benefit of U.S. Provisional Application No. 63/210,280, filed Jun. 14, 2021, which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Apr. 22, 2022, is named 0662WO_SL.txt and is 54,103 bytes in size.

### BACKGROUND

Complement-mediated injury has been implicated in the pathology of dermatomyositis (DM), a disorder characterized by distinct skin rashes and muscle weakness (see, e.g., Dalakas M C, N. Engl. J Med. 2015; 372(18):1734-1747; Isak V, et a., J. Dermatolog. Treat. 2018; 29(5):450-459; and Mahil S. et al., Br. J. Hosp. Med. (Lond). 2012; 73(2): C18-22). Patients with DM typically present with proximal muscle weakness and cutaneous manifestations that develop over weeks to months (see, e.g., Selva-O'Callaghan A, et al., The Lancet Neurology, 2018; 17(9):816-828). Muscle biopsies from patients with DM reveal the presence of membrane attack complex (MAC) on the endothelial cells of the endomysial capillaries (see, e.g., Dalakas M C, et al., Nat. Rev. Neurol. 2020; 16(11):601-617 and Kissel J T, et al., N. Engl. J. Med. 1986; 314(6):329-334). Currently, there are only 3 therapies approved for the treatment of DM: azathioprine, corticotropin injections, and glucocorticoids. Despite therapy, however, about a about a third of patients with DM are left with mild to severe disability (see, e.g., Dalakas M C, et al., Lancet. 2003; 362(9388):971-982) and half of patients considered to be stable do not return to previous levels of work (see, e.g., Marie I, et al., J. Rheumatol. 2001; 28(10):2230-2237). Accordingly, it is an object of the present disclosure to provide improved methods for treating patients with DM.

### SUMMARY

The instant disclosure is based, in part, on the insight that treating patients with ravulizumab (ALXN1210; ULTOMIRIS) will attain inhibition of terminal complement (C5) and concomitantly reduce terminal complement-mediated damage of skin tissues. The inventors' insight is based, in part, on their knowledge of pharmacodynamic (PD) effects of ravulizumab on terminal complement in vivo and identification of the involvement of membrane attack complex (MAC) in the pathophysiology of dermatomyositis (DM). The disclosure specifically relates to a new therapeutic role of ravulizumab in improving the clinical manifestations of DM in patients, particularly, the effectiveness of ravulizumab in reducing muscle weakness and/or cutaneous manifestations in severe and/or refractory DM patients.

Provided herein are compositions and methods for treating dermatomyositis (DM), in a human patient (e.g., an adult patient), comprising administering to the patient an anti-C5 antibody, or antigen binding fragment thereof, wherein the anti-C5 antibody, or antigen binding fragment thereof, is administered (or is for administration) according to a particular clinical dosage regimen (e.g., at a particular dose amount and according to a specific dosing schedule). The compositions and methods of the present disclosure are particularly useful in treating severe and/or refractory dermatomyositis.

An exemplary anti-C5 antibody is ravulizumab (ULTOMIRIS®) comprising the heavy and light chains having the sequences shown in SEQ ID NOs:14 and 11, respectively, or antigen binding fragments and variants thereof. In other embodiments, the antibody comprises the heavy and light chain complementarity determining regions (CDRs) or variable regions (VRs) of ravulizumab. Accordingly, in one embodiment, the antibody comprises the CDR1, CDR2 and CDR3 domains of the heavy chain variable (VH) region of ravulizumab having the sequence shown in SEQ ID NO:12, and the CDR1, CDR2 and CDR3 domains of the light chain variable (VL) region of ravulizumab having the sequence shown in SEQ ID NO:8. In another embodiment, the antibody comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively. In another embodiment, the antibody comprises VH and VL regions having the amino acid sequences set forth in SEQ ID NO:12 and SEQ ID NO:8, respectively. In another embodiment, the antibody comprises a heavy chain constant region as set forth in SEQ ID NO:13.

In another embodiment, the antibody comprises a variant human Fc constant region that binds to human neonatal Fc receptor (FcRn), wherein the variant human Fc CH3 constant region comprises Met429Leu and Asn435Ser substitutions at residues corresponding to methionine 428 and asparagine 434 of a native human IgG Fc constant region, each according to the EU numbering convention.

In another embodiment, the antibody comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a variant human Fc constant region that binds to human neonatal Fc receptor (FcRn), wherein the variant human Fc CH3 constant region comprises Met429Leu and Asn435Ser substitutions at residues corresponding to methionine 428 and asparagine 434 of a native human IgG Fc constant region, each according to the EU numbering convention.

In another embodiment, the anti-C5 antibody comprises the heavy and light chain CDRs or variable regions of the 7086 antibody (see U.S. Pat. Nos. 8,241,628 and 8,883,158). In another embodiment, the anti-C5 antibody comprises the heavy and light chain CDRs or variable regions of the 305LO5 antibody (see U.S. Pat. No. 9,765,135). In another embodiment, the anti-C5 antibody comprises the heavy and light chain CDRs or variable regions of the SKY59 antibody (Crovalimab). In another embodiment, the anti-C5 antibody comprises the heavy and light chain CDRs or variable regions of the REGN3918 antibody (Pozelimab). In another embodiment, the anti-C5 antibody comprises the heavy and light chain CDRs or variable regions of the LFG316 antibody (Tesidolumab). In another embodiment, the anti-C5 antibody comprises the heavy and light chain CDRs or variable regions of eculizumab or a biosimilar antibody thereof (e.g., ABP 959; SB12 or Elizaria).

In one embodiment, the dose of the anti-C5 antibody, or antigen binding fragment thereof, is based on the weight of the patient. In one embodiment, for example, 900 mg, 1200 mg, 2400 mg, 2700 mg, 3000 mg, 3300 mg, or 3600 mg of the anti-C5 antibody, or antigen binding fragment thereof, is administered based on the weight of the patient. In another embodiment, 900 mg and/or 2100 mg of the anti-C5 antibody, or antigen binding fragment thereof, is administered to a patient weighing <30 kg. In another embodiment, 1200 mg and/or 2700 mg of the anti-C5 antibody, or antigen binding fragment thereof, is administered to a patient weighing ≥30 to <40 kg. In another embodiment, 2400 mg and/or 3000 mg of the anti-C5 antibody, or antigen binding fragment thereof, is administered to a patient weighing ≥40 to <60 kg. In another embodiment, 2700 mg and/or 3300 mg of the anti-C5 antibody, or antigen binding fragment thereof, is administered to a patient weighing ≥60 to <100 kg. In another embodiment, 3000 mg and/or 3600 mg of the anti-C5 antibody, or antigen binding fragment thereof, is administered to a patient weighing ≥100 kg. In certain embodiments, dosage regimens are adjusted to provide the optimum desired response (e.g., an effective response).

In another embodiment, a method of treating a human patient with DM is provided, the method comprising administering to the patient an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody or antigen binding fragment thereof is administered:
(a) at a loading dose of 900 mg, followed by a maintenance dose of 2100 mg two weeks later and then once every eight weeks thereafter to a patient weighing <30 kg;
(b) at a loading dose of 1200 mg, followed by a maintenance dose of 2700 mg two weeks later and then once every eight weeks thereafter to a patient weighing ≥30 to <40 kg;
(c) at a loading dose of 2400 mg, followed by a maintenance dose of 3000 mg two weeks later and then once every eight weeks thereafter to a patient weighing ≥40 to <60 kg;
(d) at a loading dose of 2700 mg, followed by a maintenance dose of 3300 mg two weeks later and then once every eight weeks thereafter to a patient weighing ≥60 to <100 kg; or
(e) at a loading dose of 3000 mg, followed by a maintenance dose of 3600 mg two weeks later and then once every eight weeks thereafter to a patient weighing ≥100.

In another embodiment, a method of treating a human patient with DM is provided, the method comprising administering to the patient an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody or antigen binding fragment thereof is administered:
(a) at a loading dose of 900 mg on Day 1, followed by a maintenance dose of 2100 mg on Day 15 and then once every eight weeks thereafter to a patient weighing <30 kg;
(b) at a loading dose of 1200 mg on Day 1, followed by a maintenance dose of 2700 mg on Day 15 and then once every eight weeks thereafter to a patient weighing ≥30 to <40 kg;
(c) at a loading dose of 2400 mg on Day 1, followed by a maintenance dose of 3000 mg on Day 15 and then once every eight weeks thereafter to a patient weighing ≥40 to <60 kg;
(d) at a loading dose of 2700 mg on Day 1, followed by a maintenance dose of 3300 mg on Day 15 and then once every eight weeks thereafter to a patient weighing ≥60 to <100 kg; or
(e) at a loading dose of 3000 mg on Day 1, followed by a maintenance dose of 3600 mg on Day 15 and then once every eight weeks thereafter to a patient weighing ≥100.

In another embodiment, a method of treating a human patient with DM is provided, the method comprising administering to the patient an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering, wherein the anti-C5 antibody or antigen binding fragment thereof is administered:
(a) at a loading dose of 900 mg, followed by a maintenance dose of 2100 mg two weeks later and then once every eight weeks thereafter to a patient weighing <30 kg;
(b) at a loading dose of 1200 mg, followed by a maintenance dose of 2700 mg two weeks later and then once every eight weeks thereafter to a patient weighing ≥30 to <40 kg;
(c) at a loading dose of 2400 mg, followed by a maintenance dose of 3000 mg two weeks later and then once every eight weeks thereafter to a patient weighing ≥40 to <60 kg;
(d) at a loading dose of 2700 mg, followed by a maintenance dose of 3300 mg two weeks later and then once every eight weeks thereafter to a patient weighing ≥60 to <100 kg; or
(e) at a loading dose of 3000 mg, followed by a maintenance dose of 3600 mg two weeks later and then once every eight weeks thereafter to a patient weighing ≥100.

In another embodiment, a method of treating a human patient with DM is provided, the method comprising administering to the patient an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering, wherein the anti-C5 antibody or antigen binding fragment thereof is administered:
(a) at a loading dose of 900 mg on Day 1, followed by a maintenance dose of 2100 mg on Day 15 and then once every eight weeks thereafter to a patient weighing <30 kg;
(b) at a loading dose of 1200 mg on Day 1, followed by a maintenance dose of 2700 mg on Day 15 and then once every eight weeks thereafter to a patient weighing ≥30 to <40 kg;
(c) at a loading dose of 2400 mg on Day 1, followed by a maintenance dose of 3000 mg on Day 15 and then once every eight weeks thereafter to a patient weighing ≥40 to <60 kg;
(d) at a loading dose of 2700 mg on Day 1, followed by a maintenance dose of 3300 mg on Day 15 and then once every eight weeks thereafter to a patient weighing ≥60 to <100 kg; or
(e) at a loading dose of 3000 mg on Day 1, followed by a maintenance dose of 3600 mg on Day 15 and then once every eight weeks thereafter to a patient weighing ≥100.

In another embodiment, a method of treating a human patient with DM is provided, wherein the anti-C5 antibody is administered to a patient weighing <30 kg at a loading dose of 900 mg, followed by a maintenance dose of 2100 mg two weeks later and then once every eight weeks thereafter.

In another embodiment, a method of treating a human patient with DM is provided, wherein the anti-C5 antibody is administered to a patient weighing ≥30 to <40 kg at a loading dose of 1200 mg, followed by a maintenance dose of 2700 mg two weeks later and then once every eight weeks thereafter.

In another embodiment, a method of treating a human patient with DM is provided, wherein the anti-C5 antibody is administered to a patient weighing ≥40 to <60 kg at a loading dose of 2400 mg, followed by a maintenance dose of 3000 mg two weeks later and then once every eight weeks thereafter.

In another embodiment, a method of treating a human patient with DM is provided, wherein the anti-C5 antibody is administered to a patient weighing ≥60 to <100 kg at a loading dose of 2700 mg, followed by a maintenance dose of 3300 mg two weeks later and then once every eight weeks thereafter.

In another embodiment, a method of treating a human patient with DM is provided, wherein the anti-C5 antibody is administered to a patient weighing ≥100 kg at a loading dose of 3000 mg on Day 1, followed by a maintenance dose of 3600 mg on Day 15 and then once every eight weeks thereafter.

In another embodiment, a method of treating a human patient with DM is provided, wherein the anti-C5 antibody is administered to a patient weighing <30 kg at a loading dose of 900 mg on Day 1, followed by a maintenance dose of 2100 mg on Day 15 and then once every eight weeks thereafter.

In another embodiment, a method of treating a human patient with DM is provided, wherein the anti-C5 antibody is administered to a patient weighing ≥30 to <40 kg at a loading dose of 1200 mg on Day 1, followed by a maintenance dose of 2700 mg on Day 15 and then once every eight weeks thereafter.

In another embodiment, a method of treating a human patient with DM is provided, wherein the anti-C5 antibody is administered to a patient weighing ≥40 to <60 kg at a loading dose of 2400 mg on Day 1, followed by a maintenance dose of 3000 mg on Day 15 and then once every eight weeks thereafter.

In another embodiment, a method of treating a human patient with DM is provided, wherein the anti-C5 antibody is administered to a patient weighing ≥60 to <100 kg at a loading dose of 2700 mg on Day 1, followed by a maintenance dose of 3300 mg on Day 15 and then and then once every eight weeks thereafter.

In another embodiment, a method of treating a human patient with DM is provided, wherein the anti-C5 antibody is administered to a patient weighing ≥100 kg at a loading dose of 3000 mg, followed by a maintenance dose of 3600 mg on Day 15 and then once every eight weeks thereafter.

In one embodiment, the patient has not previously been treated with eculizumab. In another embodiment, the patient has previously been treated with eculizumab.

In another aspect, the treatment regimens described are sufficient to maintain particular serum trough concentrations of the anti-C5 antibody or antigen binding fragment thereof. In one embodiment, for example, the treatment regimen maintains a serum trough concentration of the anti-C5 antibody or antigen binding fragment thereof of 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395 or 400 μg/mL or greater. In one embodiment, the treatment regimen maintains a serum trough concentration of the anti-C5 antibody or antigen binding fragment thereof of 100 μg/mL or greater, 150 μg/mL or greater, 200 μg/mL or greater, 250 μg/mL or greater, or 300 μg/mL or greater. In another embodiment, the treatment maintains a serum trough concentration of the anti-C5 antibody or antigen binding fragment thereof of between 100 μg/mL and 200 μg/mL. In another embodiment, the treatment maintains a serum trough concentration of the anti-C5 antibody or antigen binding fragment thereof of about 175 μg/mL.

In another embodiment, to obtain an effective response, the anti-C5 antibody is administered to the patient in an amount and with a frequency to maintain at least 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg or 260 μg of antibody per milliliter of the patient's blood. In another embodiment, the anti-C5 antibody is administered to the patient in an amount and with a frequency to maintain between 50 μg and 250 μg of antibody per milliliter of the patient's blood. In another embodiment, the anti-C5 antibody is administered to the patient in an amount and with a frequency to maintain between 100 μg and 200 μg of antibody per milliliter of the patient's blood. In another embodiment, the anti-C5 antibody is administered to the patient in an amount and with a frequency to maintain about 175 μg of antibody per milliliter of the patient's blood.

In another embodiment, to obtain an effective response, the anti-C5 antibody is administered to the patient in an amount and with a frequency to maintain a minimum free C5 concentration. In one embodiment, for example, the anti-C5 antibody is administered to the patient in an amount and with a frequency to maintain a free C5 concentration of 0.5 μg/mL or less (e.g., 0.4 μg/mL, 0.3 μg/mL, 0.2 μg/mL, or 0.1 μg/mL or less).

The anti-C5 antibodies, or antigen binding fragments thereof, can be administered to a patient by any suitable means. In one embodiment, the antibodies are formulated for intravenous administration.

The efficacy of the treatment methods provided herein can be assessed using any suitable means. In one embodiment, the treatment results in a shift towards normal levels of soluble C5b-9.

In another embodiment, the treatment results in a shift towards normal levels of myositis-specific autoantibodies (e.g., anti-melanoma differentiation-associated protein 5 antibodies (anti-MDA5 antibodies), anti-nuclear matrix protein 2 antibodies (anti-NXP2/MJ antibodies), and/or anti-synthetase/Jo 1 antibodies).

In another embodiment, the treatment results in a shift towards normal levels of muscle enzymes (e.g., creatine kinase (CK), aldolase, lactate dehydrogenase (LDH), alanine aminotransferase (ALT), and aspartate aminotransferase (AST)).

In another embodiment, the treatment results in an improvement in the patient as assessed by a International Myositis Assessment and Clinical Studies Total Improvement Scale (IMACS-TIS), compared to baseline. In one embodiment, the treatment results in an at least ≥20, 25, 30, 35, 40, 45, 50, 55, or 60-point Total Improvement Score (TIS) as assessed by an IMACS-TIS score, compared to baseline.

In another embodiment, the treatment results in an improvement in the patient as assessed by a Cutaneous Dermatomyositis Disease Area and Severity Index (CDASI), compared to baseline.

In another embodiment, the treatment results in an ≥7, 8, 9, 10, 11, or 12-point improvement in the patient as assessed by a CDASI, compared to baseline.

In another embodiment, the treatment results in an improvement in the patient as assessed by a European Quality of Life Health 5-item questionnaire dimensions 5 level (EQ-5D-5L) score, compared to baseline.

In another embodiment, the treatment results in an improvement in the patient as assessed by a Patient-reported Outcomes Measurement Information System (PROMIS), compared to baseline.

In another embodiment, the treatment results in an improvement in the patient as assessed by a Short Form Health Survey (36 questions version) (SF 36), compared to baseline.

In another embodiment, the treatment results in an improvement in the patient as assessed by a Dermatomyositis Disease Symptom Questionnaire (DM-DSQ), compared to baseline.

In another embodiment, the treatment results in an improvement in the patient as assessed by a 30-Second Chair Stand Test (30s CST), compared to baseline.

In another embodiment, the treatment results in a decrease in the patient's detectable rash as assessed by photographic analysis, compared to baseline. In one embodiment, the treatment results in a 25%, 30%, 40%, 45%, 50%, 55%, 60%, 65%, 70%, 75%, 80%, 85%, 90%, 95%, 98%, 99%, or 100% decrease in the patient's detectable rash as assessed by photographic analysis, compared to baseline.

In another embodiment, the treatment results in an improvement in the patient as assessed by a 5D-itch scale, compared to baseline.

In another embodiment, the treatment results in an improvement in the patient as assessed by a handheld dynamometry performance analysis, compared to baseline.

In another embodiment, the treatment results in an improvement in the patient as assessed by a Functional Assessment of Chronic Therapy (FACIT)-Fatigue score, compared to baseline.

In another embodiment, the treatment results in an improvement in the patient as assessed by a manual muscle testing subset of 8 muscles (MMT8), compared to baseline.

In another embodiment, the treatment results in an improvement in the patient as assessed by a Myositis Disease Activity Assessment Tool (MDAAT), compared to baseline.

In another embodiment, the treatment results in an improvement in the patient as assessed by a Physician Global Activity Assessment, compared to baseline.

In another embodiment, the treatment results in an improvement in the patient as assessed by a Patient Global Activity Assessment, compared to baseline.

In another embodiment, the treatment results in an improvement in the patient as assessed by a Health Assessment Questionnaire (HAQ), compared to baseline.

In another embodiment, the treatment results in an improvement in the patient as assessed by a Cutaneous Dermatomyositis Activity Physician's Global Assessment, compared to baseline.

In another embodiment, the treatment results in a reduction or cessation in the patient's rash and/or muscle weakness.

In another embodiment, the treatment results in terminal complement inhibition.

In another embodiment, the treatment results in a reduction in adverse events.

In another embodiment, the patient has an MMT-8 of ≤142/150 and two or more of the following prior to treatment:
a) a Patient Global Activity assessment of ≥2.0 cm on a 10 cm VAS
b) a Physician Global Activity assessment of 2.0 cm on a 10 cm VAS
c) a HAQ disability index with 0.25
d) elevation of at least one muscle enzyme at 1.3 times the upper limit of normal (ULN); and/or
e) a global extramuscular disease activity score with 2.0 cm on a 10 cm visual analog scale (VAS).

In one embodiment, the global extramuscular disease activity score is based on assessments of activity scores on the constitutional, cutaneous, skeletal, gastrointestinal, pulmonary, and cardiac scales of the MDAAT.

In another embodiment, the patient has one or more of the following prior to treatment:
a) muscle or skin biopsy with evidence of active pathological findings of DM within last 6 months prior to or upon initiating treatment;
b) electromyography evidence of active myositis within the last 6 months prior to or upon initiating treatment;
c) magnetic resonance imaging (MRI) muscle evidence of active myositis within the last 6 months prior to or upon initiating treatment;
d) at least one muscle enzyme (e.g., CK, aldolase, LDH, ALT, and/or AST) in an IMACS panel ≥2 times ULN prior to or upon initiating treatment; and/or
e) active DM skin rash characterized by inflammatory changes (CDASI Activity Score ≥7) prior to or upon initiating treatment.

In some embodiments, the anti-C5 antibody, or antigen binding fragment thereof, is administered alone. In other embodiments, the anti-C5 antibody, or antigen binding fragment thereof, is administered in combination with one or more additional therapeutic agents (e.g., simultaneously or separately). In one embodiment, the treatment further comprises administering Azathioprine. In another embodiment, the treatment further comprises administering Cyclosporine. In another embodiment, the treatment further comprises administering Glucocorticoid. In another embodiment, the treatment further comprises administering intramuscular glucocorticoids. In another embodiment, the treatment further comprises administering Hydroxychloroquine. In another embodiment, the treatment further comprises administering Leflunomide. In another embodiment, the treatment further comprises administering Methotrexate. In another embodiment, the treatment further comprises administering Mycophenolate mofetil/mycophenolic acid. In another embodiment, the treatment further comprises administering Sulfasalazine. In another embodiment, the treatment further comprises administering an antihistamine. In another embodiment, the treatment further comprises administering ibuprofen. In another embodiment, the treatment further comprises administering acetaminophen. In another embodiment, the treatment further comprises administering anti-pruritics. In another embodiment, the treatment further comprises administering a topical steroid. In another embodiment, the treatment further comprises administering vitamin B12. In another embodiment, the treatment further comprises administering vitamin E. In another embodiment, the treatment further comprises administering creatine. In another embodiment, the treatment further comprises administering coenzyme Q10. In another embodiment, the treatment further comprises administering a biotin supplement.

In one embodiment, the patient has not previously taken or is not taking any of the following upon initiating or during treatment: intravenous immunoglobulin (IVIg), subcutaneous immunoglobulin (SCIg), IV glucocorticoids, corticotropin injection, cyclophosphamide, rituximab, infliximab, adalimumab, etanercept, tofacitinib, ruxolitinib, or anakinra.

Further provided are kits that include a pharmaceutical composition containing an anti-C5 antibody, or antigen binding fragment thereof, such as ravulizumab, and a pharmaceutically acceptable carrier, in a therapeutically effective amount adapted for use in the methods described herein, optionally together with background therapy. In one embodiment, the kit comprises: (a) a dose of an anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and (b) instructions for using the anti-C5 antibody, or antigen binding fragment thereof, in any of the methods described herein.

Also provided herein are anti-C5 antibodies, or antigen binding fragments thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8, wherein the anti-C5 antibody, or antigen binding fragment thereof, is administered:
(a) at a loading dose of 900 mg, followed by a maintenance dose of 2100 mg two weeks later and then once every eight weeks thereafter to a patient weighing <30 kg;
(b) at a loading dose of 1200 mg, followed by a maintenance dose of 2700 mg two weeks later and then once every eight weeks thereafter to a patient weighing ≥30 to <40 kg;
(c) at a loading dose of 2400 mg, followed by a maintenance dose of 3000 mg two weeks later and then once every eight weeks thereafter to a patient weighing ≥40 to <60 kg;
(d) at a loading dose of 2700 mg, followed by a maintenance dose of 3300 mg two weeks later and then once every eight weeks thereafter to a patient weighing ≥60 to <100 kg; or
(e) at a loading dose of 3000 mg, followed by a maintenance dose of 3600 mg two weeks later and then once every eight weeks thereafter to a patient weighing ≥100.

In some embodiments, the disclosure relates to a composition, e.g., pharmaceutical composition or a medicament, comprising an effective amount of an anti-C5 antibody or an antigen binding fragment thereof, comprising heavy chain complementarity determining regions (HCDRs) comprising HCDR1, HCDR2 and HCDR3 sequences as set forth in SEQ ID NOs:19, 18 and 3, respectively, and light chain complementarity determining regions (LCDRs) comprising LCDR1, LCDR2 and LCDR3 sequences as set forth in SEQ ID NOs:4, and 6, respectively, for use in the treatment of DM in a human patient, wherein the composition optionally comprises background therapy for treating DM. Specifically, provided herein are compositions comprising effective amounts of ravulizumab (ULTOMIRIS®) or the antigen-binding fragment thereof, for treatment of DM in a human patient. In some embodiments, the effective amount comprises use of the dosages and scheduling described herein for the anti-C5 antibody, e.g., ravulizumab.

In further embodiments, the disclosure relates to use of an effective amount of an anti-C5 antibody, or antigen binding fragment thereof, comprising heavy chain complementarity determining regions (HCDRs) comprising HCDR1, HCDR2 and HCDR3 sequences as set forth in SEQ ID NOs:19, 18 and 3, respectively, and light chain complementarity determining regions (LCDRs) comprising LCDR1, LCDR2 and LCDR3 sequences as set forth in SEQ ID NOs:4, 5 and 6, respectively, in the manufacture of a composition, e.g., pharmaceutical composition or a medicament, for treating DM in a human patient, wherein the composition optionally comprises background therapy for the treatment of DM. Specifically, provided herein are use of an effective amount of ravulizumab (ULTOMIRIS®) or the antigen-binding fragment thereof, in the manufacture of a composition, e.g., pharmaceutical composition or a medicament, for treating DM in a human patient. In some embodiments, the effective amount comprises use of the dosages and scheduling described herein for the anti-C5 antibody, e.g., ravulizumab, optionally together with dosages and scheduling of the background therapy.

### BRIEF DESCRIPTION OF THE DRAWINGS

FIG. 1 is a schematic depicting the overall study design.

FIG. 2 is a sample Physician Global Activity Assessment (IMACS Form 02).

FIG. 3 is a sample Patient/Parent Global Activity Assessment (IMACS Form 03).

FIG. 4 sets forth a sample Functional Assessment of Chronic Illness Therapy-Fatigue (FACIT-Fatigue) Questionnaire.

FIG. 5 sets forth a sample Dermatomyositis Disease Symptoms Questionnaire (DM-DSQ).

FIGS. 6A-6F set forth the Schedule of Activities for Part A (screening through end of the randomized controlled period).

FIGS. 7A-7F set forth the Schedule of Activities for Part B (screening through end of the randomized controlled period).

FIGS. 8A-8G set forth the Schedule of Activities for the Open-Label Extension Period for Parts A and B.

### DETAILED DESCRIPTION

### I. Definitions

As used herein, the term “subject” or “patient” is a human patient (e.g., a patient having DM).

As used herein, the term “pediatric” patient is a human patient that has been classified by a physician or caretaker as belonging to a non-adult category and can include, e.g., newborn (both preterm and of term), infants, children, and adolescents. Typically, pediatric patients are patients under 18 years of age (<18 years of age).

As used herein, the term “adult” patient is a human patient that has been classified by a physician or caretaker as such, e.g., one who is not a newborn, infant, child or adolescent, e.g., based on age, developmental status, physiological features, etc. Typically, adult patients are patients who are 18 years of age or older (>18 years of age).

As used herein, Dermatomyositis (DM) is an autoimmune idiopathic inflammatory disease characterized by distinct skin rashes and muscle weakness (see, e.g., Bendewald M J, et al., Archives of dermatology. 2010; 146(1):26-30; Bohan A, et al., N. Engl. J Med. 1975; 292(7):344-347; Dalakas M C, N. Engl. J. Med. 2015; 372(18):1734-1747; Isak V. et al., J. Dermatolog. Treat. 2018; 29(5):450-459; and Mahil S, et al., Br. J. Hosp. Med. (Lond). 2012; 73(2):C18-22). Patients with DM typically present with proximal muscle weakness and cutaneous manifestations that develop over weeks to months (see, e.g., Selva-O'Callaghan A, et al., The Lancet Neurology. 2018; 17(9):816-828). DM affects both children (juvenile DM) and adults, and women more than men (see, e.g., Dalakas M C, et al., Lancet; 2003; 362(9388):971-982). In the US, the incidence of DM has been estimated as 9.63 per million with a bimodal distribution peaking between the ages of 5 to 15 and 50 to 60 years (see, e.g., Bendewald M J, et al., Archives of dermatology. 2010; 146(1):26-30); Bohan A, Peter J B., N. Engl. J Med. 1975; 292(7):344-347; Isak V, et al., J Dermatolog. Treat. 2018; 29(5):450-459). Patients with DM generally develop a slow weakening of muscles that make everyday tasks like buttoning or holding objects, combing their hair, rising from a chair, climbing steps, or lifting objects difficult (see, e.g., Dalakas M C., N. Engl. J Med. 2015; 372(18):1734-1747; and Dalakas M C, Lancet. 2003; 362(9388):971-982).

Organ systems, such as vascular, pulmonary, gastrointestinal, and cardiac systems may be involved, with rare cases of interstitial lung disease potentially being fatal (see, e.g., Ernste F C, et al., Mayo. Clin. Proc. 2013; 88(1):83-105). Adult patients with DM have a 9% to 32% increased risk of cancer during the first 3 to 5 years after DM diagnosis (see, e.g., Dalakas M C., N. Engl. J. Med. 2015; 372(18):1734-1747). The most common cancers in women with DM are breast and ovarian cancers; whereas, lung and prostate cancers are the most common in men with DM (see, e.g., Ernste F C, et al., Mayo. Clin. Proc. 2013; 88(1):83-105).

Currently, azathioprine has been approved for the treatment of DM in the EU. In the US, glucocorticoids and corticotropin injections (Acthar® Gel) are the only available medications approved for the treatment of DM (see, e.g., Isak V, et al., J Dermatolog Treat. 2018; 29(5):450-459). Management of DM consists of glucocorticoids alone or in combination with immunosuppressive/immunomodulatory therapies (ISTs), including rituximab and IV immunoglobulin (IVIg) (see, e.g., Dalakas M C., N. Engl. J Med. 2015; 372(18):1734-1747). Long-term use of corticosteroids can lead to deleterious complications such as osteoporosis, compression fractures, and avascular necrosis, and use of ISTs can increase the risk of serious infections and are associated with tolerability issues as well as hepatotoxicity (see, e.g., Chandra T, et al., Expert Opin. Emerg. Drugs. 2020:1-16; and Ernste F C, et al., Mayo. Clin. Proc. 2013; 88(1):83-105). Furthermore, despite therapy, about a third of patients with DM are left with mild to severe disability (see, e.g., Dalakas M C, et al., Lancet. 2003; 362(9388):971-982) and half of patients considered to be stable do not return to previous levels of work (see, e.g., Marie I, et al., J Rheumatol. 2001; 28(10):2230-2237). With available therapies, 5-year mortality has been reduced, but is still around 25% (see, e.g., Rider L G, et al., JAMA. 2011a; 305(2):183-190).

In summary, as there are currently a limited number of treatment options available for patients with DM (especially patients with severe and/or refractory DM), many clinical treatment decisions are made empirically and without adequate clinical data. Therefore, there still exists a high unmet medical need for efficacious and safe treatment options for this disease.

Chronic activation of the immune system by environmental risk factors in individuals with a genetic predisposition is thought to be the underlying cause of DM (see, e.g., Rider L G, et al., JAMA. 2011a; 305(2):183-190). Environmental exposures, including streptococcus, echovirus, human growth hormone, interferon α, interferon γ, bovine collagen implants, physical exertion, and ultraviolet radiation, have been implicated in the development of DM. Genetic risk factors associated with susceptibility of individuals to DM include gene polymorphisms known to regulate late responses to environmental agents (e.g., polymorphisms of human leukocyte antigen [HLA], and cytokine and immunoglobulin genes).

The pathogenesis of DM is thought to be triggered when autoantibodies directed against the endothelium of endomysial capillaries activate complement component 3 (C3) disability (see, e.g., Dalakas M C, et al., Lancet. 2003; 362(9388):971-982) leading to complement-mediated injury against endothelial cells in the muscle disability (see, e.g., Dalakas M C, et al., Lancet. 2003; 362(9388):971-982; and Mahil S, et al., Br. J. Hosp. Med. (Lond). 2012; 73(2):C18-22), as well as MAC deposition in skin and muscle tissue (see, e.g., Magro C M, et al., J Cutan. Pathol. 1997; 24(9):543-552). This hypothesis is supported by activation of the complement system as an early disease manifestation of DM (see, e.g., Pinal-Fernandez I, et al., J Rheumatol. 2015; 42(8):1448-1454).

The complement pathways (classical, alternative, and lectin) all converge at C5. The activation of C5 cleaves the protein into complement components 5a and 5b (C5a and C5b); ravulizumab robustly blocks this activation. C5a is one of the strongest chemoattractants and pro inflammatory modulators (see, e.g., Harris C L, et al., Mol. Immunol. 2018; 102:89-119), recruiting B and T cells, macrophages and other immune cells to the inflammation site. These inappropriately activated immune cells release cytokines, which further potentiate the abnormal immune response with autoantibody formation and further tissue damage. In DM, activated complement C5a has been shown to up-regulate adhesion molecule expression after binding to endothelial cells, which may lead to cytokine induction (see, e.g., Sallum A M, et al., Autoimmun. Rev. 2006; 5(2):93-100).

C5b participates in the formation of the terminal complement (C5b-9) MAC (see, e.g., Tegla C A, et al., Immunol. Res. 2011; 51(1):45-60). The MACs are deposited on capillaries and induce perivascular inflammation in muscle fibers (see, e.g., Dalakas M C., N. Engl. J. Med. 2015; 372(18):1734-1747; and Dalakas M C, et al., Lancet. 2003; 362(9388):971-982). The MAC in DM causes necrosis, reduction of the density of endomysial capillaries, ischemia, and muscle fiber destruction (see, e.g., Dalakas M C., N. Engl. J. Med. 2015; 372(18):1734-1747; and Pinal-Femandez I, et al., J Rheumatol. 2015; 42(8):1448-1454). Microscopic lesions in muscle fibers lead to reduced blood flow and perifascicular atrophy (see, e.g., Dalakas M C., N. Engl. J Med. 2015; 372(18):1734-1747) which is a highly specific feature of muscle biopsies in patients with DM (specificity >90%) (see, e.g., Suarez-Calvet X, et al., Arthritis. Res. Ther. 2017; 19(1):174)

As used herein, “effective treatment” refers to treatment producing a beneficial effect, e.g., amelioration of at least one symptom of a disease or disorder. A beneficial effect can take the form of an improvement over baseline, e.g., an improvement over a measurement or observation made prior to initiation of therapy according to the method. Effective treatment may refer to alleviation of at least one symptom of DM (e.g., a violet-colored or dusky red rash develops, most commonly on the face, eyelids, knuckles, elbows, knees, chest and back) and/or muscle weakness (e.g., progressive muscle weakness in the muscles closest to the trunk, such as those in the hips, thighs, shoulders, upper arms and neck). Effective treatment may refer to alleviation of at least one symptom of DM (e.g., distinctive rash and/or muscle weakness).

The term “effective amount” refers to an amount of an agent that provides the desired biological, therapeutic and/or prophylactic result. That result can be reduction, amelioration, palliation, lessening, delaying and/or alleviation of one or more of the signs, symptoms or causes of a disease, or any other desired alteration of a biological system. In one example, an “effective amount” is the amount of anti-C5 antibody, or antigen binding fragment thereof, clinically proven to alleviate at least one symptom of DM (e.g., distinctive rash and/or muscle weakness). An effective amount can be administered in one or more administrations.

As used herein, the term “loading dose” refers to the first dose administered (e.g., during an administration cycle).

As used herein, the terms “maintenance” and “maintenance phase” are used interchangeably and refer to the second phase of treatment. In certain embodiments, treatment is continued as long as clinical benefit is observed or until unmanageable toxicity or disease progression occurs.

As used herein, the term “serum trough level” refers to the lowest level that the agent (e.g., the anti-C5 antibody, or antigen binding fragment thereof) or medicine is present in the serum. In contrast, a “peak serum level,” refers to the highest level of the agent in the serum. The “average serum level,” refers to the mean level of the agent in the serum over time.

The term “antibody” describes a polypeptide comprising at least one antibody-derived antigen binding site (e.g., VH/VL region or Fv, or CDR). Antibodies include known forms of antibodies, e.g., the antibody can be a human antibody, a humanized antibody, a bispecific antibody or a chimeric antibody. The antibody also can be a Fab, Fab′2, ScFv, SMIP, Affibody®, nanobody or a single-domain antibody. The antibody also can be of any of the following isotypes: IgG1, IgG2, IgG3, IgG4, IgM, IgA1, IgA2, IgAsec, IgD, IgE or combinations thereof. The antibody can be a naturally occurring antibody or an antibody that has been altered by a protein engineering technique (e.g., by mutation, deletion, substitution, conjugation to a non-antibody moiety). An antibody can include, for example, one or more variant amino acids (compared to a naturally occurring antibody) that change a property (e.g., a functional property) of the antibody. Numerous such alterations are known in the art that affect, e.g., half-life, effector function, and/or immune responses to the antibody in a patient. The term antibody also includes artificial or engineered polypeptide constructs that comprise at least one antibody-derived antigen binding site.

### II. Anti-C5 Antibodies

Anti-C5 antibodies described herein bind to complement component C5 (e.g., human C5) and inhibit the cleavage of C5 into fragments C5a and C5b. As described above, such antibodies also have, for example, improved pharmacokinetic properties relative to other anti-C5 antibodies (e.g., eculizumab) used for therapeutic purposes.

Anti-C5 antibodies (or VH/VL domains derived therefrom) suitable for use in the methods described herein can be generated using methods known in the art. Alternatively, art recognized anti-C5 antibodies can be used. Antibodies that compete for binding to C5 with any of these art recognized antibodies or antibodies described herein can also be used.

An exemplary anti-C5 antibody is ravulizumab comprising heavy and light chains having the sequences shown in SEQ ID NOs:14 and 11, respectively, or antigen binding fragments and variants thereof. Ravulizumab (also known as ULTOMIRIS®, BNJ441 and ALXN1210) is described in WO2015134894 and U.S. Pat. No. 9,079,949, the entire teachings of which are hereby incorporated by reference. The terms ravulizumab, BNJ441, and ALXN1210 may be used interchangeably throughout this document, but all refer to the same antibody. Ravulizumab selectively binds to human complement protein C5, inhibiting its cleavage to C5a and C5b during complement activation. This inhibition prevents the release of the proinflammatory mediator C5a and the formation of the cytolytic pore-forming membrane attack complex (MAC) C5b-9 while preserving the proximal or early components of complement activation (e.g., C3 and C3b) essential for the opsonization of microorganisms and clearance of immune complexes.

In other embodiments, the antibody comprises the heavy and light chain CDRs or heavy and light chain variable regions (VH/VL) of ravulizumab. Accordingly, in one embodiment, the antibody comprises the CDR1, CDR2 and CDR3 domains of the VH region of ravulizumab having the sequence set forth in SEQ ID NO:12, and the CDR1, CDR2 and CDR3 domains of the VL region of ravulizumab having the sequence set forth in SEQ ID NO:8. In another embodiment, the antibody comprises heavy chain CDR1, CDR2 and CDR3 domains having the sequences set forth in SEQ ID NOs:19, 18 and 3, respectively, and light chain CDR1, CDR2 and CDR3 domains having the sequences set forth in SEQ ID NOs:4, 5 and 6, respectively. In another embodiment, the antibody comprises VH and VL regions having the amino acid sequences set forth in SEQ ID NO: 12 and SEQ ID NO:8, respectively. In another embodiment, the antibody comprises a heavy chain having the amino acid sequence set forth in SEQ ID: 14. In another embodiment, the antibody comprises a heavy chain constant region having the amino acid sequence set forth in SEQ ID: 15. In another embodiment, the antibody comprises a heavy chain constant region having the amino acid sequence set forth in SEQ ID: 16.

Another exemplary anti-C5 antibody is antibody BNJ421 comprising heavy and light chains having the sequences shown in SEQ ID NOs:20 and 11, respectively, or antigen binding fragments and variants thereof. BNJ421 (also known as ALXN1211) is described in WO2015134894 and U.S. Pat. No. 9,079,949, the entire teachings of which are hereby incorporated by reference.

In other embodiments, the antibody comprises the heavy and light chain CDRs or variable regions of BNJ421. Accordingly, in one embodiment, the antibody comprises the CDR1, CDR2 and CDR3 domains of the VH region of BNJ421 having the sequence set forth in SEQ ID NO:12, and the CDR1, CDR2 and CDR3 domains of the VL region of BNJ421 having the sequence set forth in SEQ ID NO:8. In another embodiment, the antibody comprises heavy chain CDR1, CDR2 and CDR3 domains having the sequences set forth in SEQ ID NOs:19, 18 and 3, respectively, and light chain CDR1, CDR2 and CDR3 domains having the sequences set forth in SEQ ID NOs:4, 5 and 6, respectively. In another embodiment, the antibody comprises VH and VL regions having the amino acid sequences set forth in SEQ ID NO:12 and SEQ ID NO:8, respectively. In another embodiment, the antibody comprises a heavy chain constant region having the amino acid sequence set forth in SEQ ID: 9.

In another embodiment, the antibody comprises a heavy chain having the amino acid sequence set forth in SEQ ID: 10.

Another exemplary anti-C5 antibody is the 7086 antibody described in U.S. Pat. Nos. 8,241,628 and 8,883,158. In one embodiment, the antibody comprises the heavy and light chain CDRs or variable regions of the 7086 antibody (see U.S. Pat. Nos. 8,241,628 and 8,883,158). In another embodiment, the antibody, or antigen binding fragment thereof, comprises heavy chain CDR1, CDR2 and CDR3 domains having the sequences set forth in SEQ ID NOs:21, 22 and 23, respectively, and light chain CDR1, CDR2 and CDR3 domains having the sequences set forth in SEQ ID NOs:24, 25 and 26, respectively. In another embodiment, the antibody, or antigen binding fragment thereof, comprises the VH region of the 7086 antibody having the sequence set forth in SEQ ID NO:27, and the VL region of the 7086 antibody having the sequence set forth in SEQ ID NO:28.

Another exemplary anti-C5 antibody is the 305LO5 antibody described in U.S. Pat. No. 9,765,135. In one embodiment, the antibody comprises the heavy and light chain CDRs or variable regions of the 305LO5 antibody. In another embodiment, the antibody, or antigen binding fragment thereof, comprises heavy chain CDR1, CDR2 and CDR3 domains having the sequences set forth in SEQ ID NOs:29, 30, and 31, respectively, and light chain CDR1, CDR2 and CDR3 domains having the sequences set forth in SEQ ID NOs:32, 33 and 34, respectively. In another embodiment, the antibody comprises the VH region of the 305LO5 antibody having the sequence set forth in SEQ ID NO:35, and the VL region of the 305LO5 antibody having the sequence set forth in SEQ ID NO:36.

Another exemplary anti-C5 antibody is the SKY59 antibody (Fukuzawa, T. et al., Sci. Rep., 7:1080, 2017). In one embodiment, the antibody comprises the heavy and light chain CDRs or variable regions of the SKY59 antibody (Crovalimab). In another embodiment, the antibody, or antigen binding fragment thereof, comprises a heavy chain comprising SEQ ID NO:37 and a light chain comprising SEQ ID NO:38.

In some embodiments, the anti-C5 antibody comprises the heavy and light chain variable regions or heavy and light chains of the REGN3918 antibody (Pozelimab). See US Patent No. 10,633,434. In some embodiments, the anti-C5 antibody, or antigen-binding fragment thereof, comprises a heavy chain variable region sequence set forth in SEQ ID NO:39 and a light chain variable region comprising the sequence set forth in SEQ ID NO:40. In some embodiments, the anti-C5 antibody, or antigen-binding fragment thereof, comprises a heavy chain sequence set forth in SEQ ID NO:41 and a light chain sequence set forth in SEQ ID NO:42.

In some embodiments, the anti-C5 antibody comprises the heavy and light chain CDRs or heavy and light chains of the LFG316 antibody (Tesidolumab).

In some embodiments, the anti-C5 antibody comprises the heavy and light chain CDRs or heavy and light chains of eculizumab or a biosimilar antibody thereof (e.g., ABP 959; SB12 or Elizaria).

In some embodiments, the antibodies comprise complementarity determining regions (CDRs) comprising variable heavy CDRs (VHCDR1-3) and variable light CDRs (VLCDR1-3). The exact boundaries of CDRs are defined differently according to different methods. In some embodiments, the positions of the CDRs or framework regions within a light or heavy chain variable domain are as defined by Kabat et al. [(1991) “Sequences of Proteins of Immunological Interest.” NIH Publication No. 91-3242, U.S. Department of Health and Human Services, Bethesda, MD]. In such cases, the CDRs can be referred to as “Kabat C DRs” (e.g., “Kabat LCDR2” or “Kabat HCDR1”). In some embodiments, the positions of the CDRs of a light or heavy chain variable region are as defined by Chothia et al. (Nature, 342:877-83, 1989). Accordingly, these regions can be referred to as “Chothia CDRs” (e.g., “Chothia LCDR2” or “Chothia HCDR3”). In some embodiments, the positions of the CDRs of the light and heavy chain variable regions can be defined by a Kabat-Chothia combined definition. In such embodiments, these regions can be referred to as “combined Kabat-Chothia CDRs.” Thomas, C. et al. (Mol. Immunol., 33:1389-401, 1996) exemplifies the identification of CDR boundaries according to Kabat and Chothia numbering schemes.

In some embodiments, an anti-C5 antibody described herein comprises a heavy chain CDR1 comprising, or consisting of, the following amino acid sequence: GHIFSNYWIQ (SEQ ID NO:19). In some embodiments, an anti-C5 antibody described herein comprises a heavy chain CDR2 comprising, or consisting of, the following amino acid sequence:

(SEQ ID NO: 18)
EILPGSGHTEYTENFKD

In some embodiments, an anti-C5 antibody described herein comprises a heavy chain variable region comprising the following amino acid sequence:

(SEQ ID NO: 12)
QVQLVQSGAE VKKPGASVKV SCKASGHIFS NYWIQWVRQA
PGQGLEWMGE ILPGSGHTEY TENFKDRVTM TRDTSTSTVY
MELSSLRSED TAVYYCARYF FGSSPNWYFD VWGQGTLVTV SS.

In some embodiments, an anti-C5 antibody described herein comprises a light chain variable region comprising the following amino acid sequence:

(SEQ ID NO: 8)
DIQMTQSPSS LSASVGDRVT ITCGASENIY GALNWYQQKP
GKAPKLLIYG ATNLADGVPS RFSGSGSGTD FTLTISSLQP
EDFATYYCQN VLNTPLTFGQ GTKVEIK.

An anti-C5 antibody described herein can, in some embodiments, comprise a variant human Fc constant region that binds to human neonatal Fc receptor (FcRn) with greater affinity than that of the native human Fc constant region from which the variant human Fc constant region was derived. The Fc constant region can, for example, comprise one or more (e.g., two, three, four, five, six, seven, or eight or more) amino acid substitutions relative to the native human Fc constant region from which the variant human Fc constant region was derived. The substitutions can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art and exemplified in the working examples. See, e.g., WO2015134894 and U.S. Pat. No. 9,079,949 the disclosures of each of which are incorporated herein by reference in their entirety.

Substitutions that enhance the binding affinity of an antibody Fc constant region for FcRn are known in the art and include, e.g., (1) the M252Y/S254T/T256E triple substitution (Dall′Acqua, W. et al., J. Biol. Chem., 281:23514-24, 2006); (2) the M428L or T250Q/M428L substitutions (Hinton, P. et al., J. Biol. Chem., 279:6213-6, 2004; Hinton, P. et al., J. Immunol., 176:346-56, 2006); and (3) the N434A or T307/E380A/N434A substitutions (Petkova, S. et al., Int. Immunol., 18:1759-69, 2006). The additional substitution pairings: P257I/Q311I, P257I/N434H and D376V/N434H (Datta-Mannan, A. et al., J Biol. Chem., 282:1709-17, 2007), the disclosures of each of which are incorporated herein by reference in their entirety.

Suitable anti-C5 antibodies for use in the methods described herein, in some embodiments, comprise a heavy chain polypeptide comprising the amino acid sequence set forth in SEQ ID NO:14 and/or a light chain polypeptide comprising the amino acid sequence set forth in SEQ ID NO:11. Alternatively, the anti-C5 antibodies for use in the methods described herein, in some embodiments, comprise a heavy chain polypeptide comprising the amino acid sequence set forth in SEQ ID NO:20 and/or a light chain polypeptide comprising the amino acid sequence set forth in SEQ ID NO: 11.

In one embodiment, the antibody binds to C5 at pH 7.4 and 25° C. (and, otherwise, under physiologic conditions) with an affinity dissociation constant (K_{D}) that is at least 0.1 (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, or 0.975) nM. In one embodiment, the antibody binds to C5 at pH 7.4 and 25° C. (and, otherwise, under physiologic conditions) with an affinity dissociation constant (K_{D}) that is about 0.5 nM. In some embodiments, the K_{D} of the anti-C5 antibody, or antigen binding fragment thereof, is no greater than 1 (e.g., no greater than 0.9, 0.8, 0.7, 0.6, 0.5, 0.4, 0.3, or 0.2) nM. In some embodiments, the antibody binds to C5 at pH 6.0 and 25° C. (and, otherwise, under physiologic conditions) with a K_{D} that is about 22 nM.

In other embodiments, the [(K_{D} of the antibody for C5 at pH 6.0 at 25° C.)/(K_{D} of the antibody for C5 at pH 7.4 at 25C)] is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500 or 8000)

Methods for determining whether an antibody binds to a protein antigen and/or the affinity for an antibody to a protein antigen are known in the art. The binding of an antibody to a protein antigen, for example, can be detected and/or quantified using a variety of techniques such as, but not limited to, Western blot, dot blot, surface plasmon resonance (SPR) detection (e.g., BIAcore system; Pharmacia Biosensor AB, Uppsala, Sweden and Piscataway, N.J.), or enzyme-linked immunosorbent assay (ELISA; Benny K. C. Lo (2004) “Antibody Engineering: Methods and Protocols,” Humana Press (ISBN: 1588290921); Johne, B. et al., J Immunol. Meth., 160:191-8, 1993; Jönsson, U. et al., Ann. Biol. Clin., 51:19-26, 1993; Jonsson, U. et al., Biotechniques, 11:620-7, 1991). In addition, methods for measuring the affinity (e.g., dissociation and association constants) are set forth in the working examples.

As used herein, the term “kₐ” refers to the rate constant for association of an antibody to an antigen. The term “k_{d}” refers to the rate constant for dissociation of an antibody from the antibody/antigen complex. And the term “K_{D}” refers to the equilibrium dissociation constant of an antibody-antigen interaction. The equilibrium dissociation constant is deduced from the ratio of the kinetic rate constants, K_{D}=kₐ/k_{d}. Such determinations can be measured, for example, at 25C or 37C (see the working examples). The kinetics of antibody binding to human C5 can be determined, for example, at pH 8.0, 7.4, 7.0, 6.5 and 6.0 via SPR on a BIAcore 3000 instrument using an anti-Fc capture method to immobilize the antibody.

In one embodiment, the anti-C5 antibody, or antigen binding fragment thereof, blocks the cleavage of C5 into C5a and C5b. Through this blocking effect, for example, the pro-inflammatory effects of C5a and the generation of the C5b-9 membrane attack complex (MAC) at the surface of a cell are inhibited.

Methods for determining whether a particular antibody described herein inhibits C5 cleavage are known in the art. Inhibition of human complement component C5 can reduce the cell-lysing ability of complement in a subject's body fluids. Such reductions of the cell-lysing ability of complement present in the body fluid(s) can be measured by methods known in the art such as, for example, by a conventional hemolytic assay such as the hemolysis assay (Kabat and Mayer (eds.), “Experimental Immunochemistry, 2^{nd} Edition,” 135-240, Springfield, IL, CC Thomas (1961), pages 135-139), or a conventional variation of that assay such as the chicken erythrocyte hemolysis method (Hillmen, P. et al., N. Engl. J. Med., 350:552-9, 2004). Methods for determining whether a candidate compound inhibits the cleavage of human C5 into forms C5a and C5b are known in the art (Evans, M. et al., Mol. Immunol., 32:1183-95, 1995). The concentration and/or physiologic activity of C5a and C5b in a body fluid can be measured, for example, by methods known in the art. For C5b, hemolytic assays or assays for soluble C5b-9 as discussed herein can be used. Other assays known in the art can also be used. Using assays of these or other suitable types, candidate agents capable of inhibiting human complement component C5 can be screened.

Immunological techniques such as, but not limited to, ELISA can be used to measure the protein concentration of C5 and/or its split products to determine the ability of an anti-C5 antibody, or antigen binding fragment thereof, to inhibit conversion of C5 into biologically active products. In some embodiments, C5a generation is measured. In some embodiments, C5b-9 neoepitope-specific antibodies are used to detect MAC formation.

Hemolytic assays can be used to determine the inhibitory activity of an anti-C5 antibody, or antigen binding fragment thereof, on complement activation. To determine the effect of an anti-C5 antibody, or antigen binding fragment thereof, on classical complement pathway-mediated hemolysis in a serum test solution in vitro, for example, sheep erythrocytes coated with hemolysin or chicken erythrocytes sensitized with anti-chicken erythrocyte antibody are used as target cells. The percentage of lysis is normalized by considering 100% lysis equal to the lysis occurring in the absence of the inhibitor. In some embodiments, the classical complement pathway is activated by a human IgM antibody, for example, as utilized in the Wieslab® Classical Pathway Complement Kit (Wieslab® COMPL CP310, Euro-Diagnostica, Sweden). Briefly, the test serum is incubated with an anti-C5 antibody, or antigen binding fragment thereof, in the presence of a human IgM antibody. The amount of C5b-9 that is generated is measured by contacting the mixture with an enzyme conjugated anti-C5b-9 antibody and a fluorogenic substrate and measuring the absorbance at the appropriate wavelength. As a control, the test serum is incubated in the absence of the anti-C5 antibody, or antigen binding fragment thereof. In some embodiments, the test serum is a C5-deficient serum reconstituted with a C5 polypeptide.

To determine the effect of an anti-C5 antibody, or antigen binding fragment thereof, on alternative pathway-mediated hemolysis, unsensitized rabbit or guinea pig erythrocytes can be used as the target cells. In some embodiments, the serum test solution is a C5-deficient serum reconstituted with a C5 polypeptide. The percentage of lysis is normalized by considering 100% lysis equal to the lysis occurring in the absence of the inhibitor. In some embodiments, the alternative complement pathway is activated by lipopolysaccharide molecules, for example, as utilized in the Wieslab© Alternative Pathway Complement Kit (Wieslab® COMPL AP330, Euro-Diagnostica, Sweden). Briefly, the test serum is incubated with an anti-C5 antibody, or antigen binding fragment thereof, in the presence of lipopolysaccharide. The amount of C5b-9 that is generated is measured by contacting the mixture with an enzyme conjugated anti-C5b-9 antibody and a fluorogenic substrate and measuring the fluorescence at the appropriate wavelength. As a control, the test serum is incubated in the absence of the anti-C5 antibody, or antigen binding fragment thereof.

In some embodiments, C5 activity, or inhibition thereof, is quantified using a CH50 eq assay. The CH50 eq assay is a method for measuring the total classical complement activity in serum. This test is a lytic assay, which uses antibody-sensitized erythrocytes as the activator of the classical complement pathway and various dilutions of the test serum to determine the amount required to give 50% lysis (CH50). The percent hemolysis can be determined, for example, using a spectrophotometer. The CH50 eq assay provides an indirect measure of terminal complement complex (TCC) formation, since the TCC themselves are directly responsible for the hemolysis that is measured. The assay is known and commonly practiced by those of skill in the art. Briefly, to activate the classical complement pathway, undiluted serum samples (e.g., reconstituted human serum samples) are added to microassay wells containing the antibody-sensitized erythrocytes to thereby generate TCC. Next, the activated sera are diluted in microassay wells, which are coated with a capture reagent (e.g., an antibody that binds to one or more components of the TCC). The TCC present in the activated samples bind to the monoclonal antibodies coating the surface of the microassay wells. The wells are washed and to each well is added a detection reagent that is detectably labeled and recognizes the bound TCC. The detectable label can be, e.g., a fluorescent label or an enzymatic label. The assay results are expressed in CH50 unit equivalents per milliliter (CH50 U Eq/mL).

Inhibition, e.g., as it pertains to terminal complement activity, includes at least a 5 (e.g., at least a 6, 7, 8, 9, 10, 15, 20, 25, 30, 35, 40, 45, 50, 55 or 60) % decrease in the activity of terminal complement in, e.g., a hemolytic assay or CH50 eq assay as compared to the effect of a control antibody (or antigen-binding fragment thereof) under similar conditions and at an equimolar concentration. Substantial inhibition, as used herein, refers to inhibition of a given activity (e.g., terminal complement activity) of at least 40 (e.g., at least 45, 50, 55, 60, 65, 70, 75, 80, 85, 90, or 95 or greater) %. In some embodiments, an anti-C5 antibody described herein contains one or more amino acid substitutions relative to the CDRs of eculizumab (i.e., SEQ ID NOs:1-6), yet retains at least 30 (e.g., at least 31, 32, 33, 34, 35, 36, 37, 38, 39, 40, 41, 42, 43, 44, 45, 46, 47, 48, 49, 50, 55, 60, 65, 70, 75, 80, 85, 90 or 95) % of the complement inhibitory activity of eculizumab in a hemolytic assay or CH50 eq assay.

An anti-C5 antibody described herein has a serum half-life in humans that is at least 20 (e.g., at least 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55) days. In another embodiment, the anti-C5 antibody described herein has a serum half-life in humans that is at least 40 days. In another embodiment, the anti-C5 antibody described herein has a serum half-life in humans that is approximately 43 days. In another embodiment, the anti-C5 antibody described herein has a serum half-life in humans that is between 39-48 days. Methods for measuring the serum half-life of an antibody are known in the art. In some embodiments, an anti-C5 antibody, or antigen binding fragment thereof, described herein has a serum half-life that is at least 20 (e.g., at least 30, 35, 40, 45, 50, 55, 60, 65, 70, 75, 80, 85, 90, 95, 100, 125, 150, 175, 200, 250, 300, 400 or 500) % greater than the serum half-life of eculizumab, e.g., as measured in one of the mouse model systems described in the working examples (e.g., the C5-deficient/NOD/scid mouse or hFcRn transgenic mouse model system).

In one embodiment, the antibody competes for binding with, and/or binds to the same epitope on C5 as an antibody described herein. The term “binds to the same epitope” with reference to two or more antibodies means that the antibodies bind to the same segment of amino acid residues, as determined by a given method. Techniques for determining whether antibodies bind to the same epitope on C5 with an antibody described herein include, for example, epitope mapping methods, such as, x-ray analyses of crystals of antigen:antibody complexes, and hydrogen/deuterium exchange mass spectrometry (HDX-MS). Other methods monitor the binding of the antibody to peptide antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, CDR2 and CDR3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the antibody that is incubated first with the target). Competing antibodies can bind to, for example, the same epitope, an overlapping epitope or to adjacent epitopes (e.g., as evidenced by steric hindrance).

Anti-C5 antibodies, or antigen-binding fragments thereof described herein, used in the methods described herein can be generated using a variety of art-recognized techniques. Monoclonal antibodies can be obtained by various techniques familiar to those skilled in the art. Briefly, spleen cells from an animal immunized with a desired antigen are immortalized, commonly by fusion with a myeloma cell (Kohler, G. & Milstein, C., Eur. J Immunol., 6:511-9, 1976)). Methods of immortalization include transformation with Epstein Barr Virus, oncogenes, or retroviruses or other methods known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences that encode a monoclonal antibody or a binding fragment thereof by screening a DNA library from human B cells (Huse, W. et al., Science, 246:1275-81, 1989).

In some embodiments, the anti-C5 antibody does not comprise eculizumab (SOLIRIS®) or an antigen-binding fragment thereof (e.g., comprising heavy and light chain complementarity determining regions (HCDR₁₋₃ and LCDR₁₋₃, respectively) of eculizumab). In some embodiments, the anti-C5 antibody is not a biosimilar of eculizumab (SOLIRIS®), e.g., ABP 959 antibody (manufactured by Amgen Inc., USA), ELIZARIA® (manufactured by Generium JNC, Russia), or SB12 (manufactured by Samsung Bioepis, Incheon, South Korea).

### III. Compositions

Also provided herein are compositions comprising an anti-C5 antibody, or antigen binding fragment thereof. In one embodiment, the composition comprises an anti-C5 antibody comprising the CDR1, CDR2 and CDR3 domains in a heavy chain variable region having the sequence set forth in SEQ ID NO:12, and the CDR1, CDR2 and CDR3 domains in a light chain variable region having the sequence set forth in SEQ ID NO:8. In another embodiment, the anti-C5 antibody comprises heavy and light chains having the sequences shown in SEQ ID NOs:14 and 11, respectively. In another embodiment, the anti-C5 antibody comprises heavy and light chains having the sequences shown in SEQ ID NOs:20 and 11, respectively.

The compositions can be formulated as a pharmaceutical solution, e.g., for administration to a subject for the treatment of DM. The pharmaceutical compositions generally include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt, sugars, carbohydrates, polyols and/or tonicity modifiers.

The compositions can be formulated according to standard methods. Pharmaceutical formulation is an established art (see, for example, Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7^{th} Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X)). In some embodiments, a composition can be formulated, for example, as a buffered solution at a suitable concentration and suitable for storage at 2-8C (e.g., 4° C.). In some embodiments, a composition can be formulated for storage at a temperature below OC (e.g., −20° C. or −80° C.). In some embodiments, the composition can be formulated for storage for up to 2 years (e.g., 1 month, 2 months, 3 months, 4 months, 5 months, 6 months, 7 months, 8 months, 9 months, 10 months, 11 months, 1 year, 112 years or 2 years) at 2-8° C. (e.g., 4° C.). Thus, in some embodiments, the compositions described herein are stable in storage for at least 1 year at 2-8° C. (e.g., 4° C.).

The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. Compositions containing a composition intended for systemic or local delivery, for example, can be in the form of injectable or infusible solutions. Accordingly, the compositions can be formulated for administration by a parenteral mode (e.g., intravenous, subcutaneous, intraperitoneal, or intramuscular injection). “Parenteral administration,” “administered parenterally” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

In some embodiments, the disclosure relates to a composition, e.g., pharmaceutical composition or a medicament, comprising an effective amount of an anti-C5 antibody or an antigen binding fragment thereof, comprising heavy chain complementarity determining regions (HCDRs) comprising HCDR1, HCDR2 and HCDR3 sequences as set forth in SEQ ID NOs:19, 18 and 3, respectively, and light chain complementarity determining regions (LCDRs) comprising LCDR1, LCDR2 and LCDR3 sequences as set forth in SEQ ID NOs:4, and 6, respectively, for use in the treatment of DM, in a human patient, wherein the effective amount comprises administration of the anti-C5 antibody, or the antigen binding fragment thereof:
(a) at a loading dose of 900 mg, followed by a maintenance dose of 2100 mg two weeks later and then once every eight weeks thereafter to a patient weighing <30 kg;
(b) at a loading dose of 1200 mg, followed by a maintenance dose of 2700 mg two weeks later and then once every eight weeks thereafter to a patient weighing ≥30 to <40 kg;
(c) at a loading dose of 2400 mg, followed by a maintenance dose of 3000 mg two weeks later and then once every eight weeks thereafter to a patient weighing ≥40 to <60 kg;
(d) at a loading dose of 2700 mg, followed by a maintenance dose of 3300 mg two weeks later and then once every eight weeks thereafter to a patient weighing ≥60 to <100 kg; or
(e) at a loading dose of 3000 mg, followed by a maintenance dose of 3600 mg two weeks later and then once every eight weeks thereafter to a patient weighing ≥100.

In other embodiments, the disclosure relates to a composition, e.g., pharmaceutical composition or a medicament, comprising an effective amount of an anti-C5 antibody or an antigen binding fragment thereof, comprising heavy chain complementarity determining regions (HCDRs) comprising HCDR1, HCDR2 and HCDR3 sequences as set forth in SEQ ID NOs:19, 18 and 3, respectively, and light chain complementarity determining regions (LCDRs) comprising LCDR1, LCDR2 and LCDR3 sequences as set forth in SEQ ID NOs:4, and 6, respectively, for use in the treatment of DM, in a human patient, wherein the effective amount comprises administration of the anti-C5 antibody, or the antigen binding fragment thereof:
(a) at a loading dose of 900 mg on Day 1, followed by a maintenance dose of 2100 mg on Day 15 and then once every eight weeks thereafter to a patient weighing <30 kg;
(b) at a loading dose of 1200 mg on Day 1, followed by a maintenance dose of 2700 mg on Day 15 and then once every eight weeks thereafter to a patient weighing ≥30 to <40 kg;
(c) at a loading dose of 2400 mg on Day 1, followed by a maintenance dose of 3000 mg on Day 15 and then once every eight weeks thereafter to a patient weighing ≥40 to <60 kg;
(d) at a loading dose of 2700 mg on Day 1, followed by a maintenance dose of 3300 mg on Day 15 and then once every eight weeks thereafter to a patient weighing ≥60 to <100 kg; or
(e) at a loading dose of 3000 mg on Day 1, followed by a maintenance dose of 3600 mg on Day 15 and then once every eight weeks thereafter to a patient weighing ≥100.

In some embodiments, the disclosure relates to a composition, e.g., pharmaceutical composition or a medicament, comprising an effective amount of an anti-C5 antibody or antigen binding fragment thereof, comprising HCDR₁₋₃ comprising SEQ ID NOs:19, 18 and 3 and LCDR₁₋₃ comprising SEQ ID NOs: 4, 5 and 6, for use in the treatment of DM in a human patient, wherein the anti-C5 antibody further comprises a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering. Particularly, the disclosure relates to a pharmaceutical composition or a medicament comprising an effective amount of ravulizumab (ULTOMIRIS®) or an antigen-binding fragment thereof, e.g., comprising the HCDR₁₋₃ and the LCDR₁₋₃ of ravulizumab, for use in the treatment of DM in a human patient.

### IV. Methods

Provided herein are methods for treating DM in a human patient, comprising administering to the patient an anti-C5 antibody, or antigen binding fragment thereof, wherein the anti-C5 antibody or antigen binding fragment thereof is administered (or is for administration) according to a particular clinical dosage regimen (e.g., at a particular dose amount and according to a specific dosing schedule).

In one embodiment, the dose of the anti-C5 antibody, or antigen binding fragment thereof, is based on the weight of the patient. In one embodiment, for example, 900 mg, 1200 mg, 2400 mg, 2700 mg, 3000 mg, 3300 mg, or 3600 mg of the anti-C5 antibody, or antigen binding fragment thereof, is administered based on the weight of the patient. In another embodiment, 900 mg and/or 2100 mg of the anti-C5 antibody, or antigen binding fragment thereof, is administered to a patient weighing <30 kg. In another embodiment, 1200 mg and/or 2700 mg of the anti-C5 antibody, or antigen binding fragment thereof, is administered to a patient weighing ≥30 to <40 kg. In another embodiment, 2400 mg and/or 3000 mg of the anti-C5 antibody, or antigen binding fragment thereof, is administered to a patient weighing ≥40 to <60 kg. In another embodiment, 2700 mg and/or 3300 mg of the anti-C5 antibody, or antigen binding fragment thereof, is administered to a patient weighing ≥60 to <100 kg. In another embodiment, 3000 mg and/or 3600 mg of the anti-C5 antibody, or antigen binding fragment thereof, is administered to a patient weighing ≥100 kg. In certain embodiments, dosage regimens are adjusted to provide the optimum desired response (e.g., an effective response).

In another embodiment, a method of treating a human patient with DM is provided, the method comprising administering to the patient an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody or antigen binding fragment thereof is administered:
(a) at a loading dose of 900 mg, followed by a maintenance dose of 2100 mg two weeks later and then once every eight weeks thereafter to a patient weighing <30 kg;
(b) at a loading dose of 1200 mg, followed by a maintenance dose of 2700 mg two weeks later and then once every eight weeks thereafter to a patient weighing ≥30 to <40 kg;
(c) at a loading dose of 2400 mg, followed by a maintenance dose of 3000 mg two weeks later and then once every eight weeks thereafter to a patient weighing ≥40 to <60 kg;
(d) at a loading dose of 2700 mg, followed by a maintenance dose of 3300 mg two weeks later and then once every eight weeks thereafter to a patient weighing ≥60 to <100 kg; or
(e) at a loading dose of 3000 mg, followed by a maintenance dose of 3600 mg two weeks later and then once every eight weeks thereafter to a patient weighing ≥100.

In another embodiment, a method of treating a human patient with DM is provided, the method comprising administering to the patient an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody or antigen binding fragment thereof is administered:
(a) at a loading dose of 900 mg on Day 1, followed by a maintenance dose of 2100 mg on Day 15 and then once every eight weeks thereafter to a patient weighing <30 kg;
(b) at a loading dose of 1200 mg on Day 1, followed by a maintenance dose of 2700 mg on Day 15 and then once every eight weeks thereafter to a patient weighing ≥30 to <40 kg;
(c) at a loading dose of 2400 mg on Day 1, followed by a maintenance dose of 3000 mg on Day 15 and then once every eight weeks thereafter to a patient weighing ≥40 to <60 kg;
(d) at a loading dose of 2700 mg on Day 1, followed by a maintenance dose of 3300 mg on Day 15 and then once every eight weeks thereafter to a patient weighing ≥60 to <100 kg; or
(e) at a loading dose of 3000 mg on Day 1, followed by a maintenance dose of 3600 mg on Day 15 and then once every eight weeks thereafter to a patient weighing ≥100.

In another embodiment, a method of treating a human patient with DM is provided, the method comprising administering to the patient an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering, wherein the anti-C5 antibody or antigen binding fragment thereof is administered:
(a) at a loading dose of 900 mg, followed by a maintenance dose of 2100 mg two weeks later and then once every eight weeks thereafter to a patient weighing <30 kg;
(b) at a loading dose of 1200 mg, followed by a maintenance dose of 2700 mg two weeks later and then once every eight weeks thereafter to a patient weighing ≥30 to <40 kg;
(c) at a loading dose of 2400 mg, followed by a maintenance dose of 3000 mg two weeks later and then once every eight weeks thereafter to a patient weighing ≥40 to <60 kg;
(d) at a loading dose of 2700 mg, followed by a maintenance dose of 3300 mg two weeks later and then once every eight weeks thereafter to a patient weighing ≥60 to <100 kg; or
(e) at a loading dose of 3000 mg, followed by a maintenance dose of 3600 mg two weeks later and then once every eight weeks thereafter to a patient weighing ≥100.

In another embodiment, a method of treating a human patient with DM is provided, the method comprising administering to the patient an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering, wherein the anti-C5 antibody or antigen binding fragment thereof is administered:
(a) at a loading dose of 900 mg on Day 1, followed by a maintenance dose of 2100 mg on Day 15 and then once every eight weeks thereafter to a patient weighing <30 kg;
(b) at a loading dose of 1200 mg on Day 1, followed by a maintenance dose of 2700 mg on Day 15 and then once every eight weeks thereafter to a patient weighing ≥30 to <40 kg;
(c) at a loading dose of 2400 mg on Day 1, followed by a maintenance dose of 3000 mg on Day 15 and then once every eight weeks thereafter to a patient weighing ≥40 to <60 kg;
(d) at a loading dose of 2700 mg on Day 1, followed by a maintenance dose of 3300 mg on Day 15 and then once every eight weeks thereafter to a patient weighing ≥60 to <100 kg; or
(e) at a loading dose of 3000 mg on Day 1, followed by a maintenance dose of 3600 mg on Day 15 and then once every eight weeks thereafter to a patient weighing ≥100.

In another embodiment, a method of treating a human patient with DM is provided, wherein the anti-C5 antibody is administered to a patient weighing <30 kg at a loading dose of 900 mg, followed by a maintenance dose of 2100 mg two weeks later and then once every eight weeks thereafter.

In another embodiment, a method of treating a human patient with DM is provided, wherein the anti-C5 antibody is administered to a patient weighing ≥30 to <40 kg at a loading dose of 1200 mg, followed by a maintenance dose of 2700 mg two weeks later and then once every eight weeks thereafter.

In another embodiment, a method of treating a human patient with DM is provided, wherein the anti-C5 antibody is administered to a patient weighing ≥40 to <60 kg at a loading dose of 2400 mg, followed by a maintenance dose of 3000 mg two weeks later and then once every eight weeks thereafter.

In another embodiment, a method of treating a human patient with DM is provided, wherein the anti-C5 antibody is administered to a patient weighing ≥60 to <100 kg at a loading dose of 2700 mg, followed by a maintenance dose of 3300 mg two weeks later and then once every eight weeks thereafter.

In another embodiment, a method of treating a human patient with DM is provided, wherein the anti-C5 antibody is administered to a patient weighing ≥100 kg at a loading dose of 3000 mg on Day 1, followed by a maintenance dose of 3600 mg on Day 15 and then once every eight weeks thereafter.

In another embodiment, a method of treating a human patient with DM is provided, wherein the anti-C5 antibody is administered to a patient weighing <30 kg at a loading dose of 900 mg on Day 1, followed by a maintenance dose of 2100 mg on Day 15 and then once every eight weeks thereafter.

In another embodiment, a method of treating a human patient with DM is provided, wherein the anti-C5 antibody is administered to a patient weighing ≥30 to <40 kg at a loading dose of 1200 mg on Day 1, followed by a maintenance dose of 2700 mg on Day 15 and then once every eight weeks thereafter.

In another embodiment, a method of treating a human patient with DM is provided, wherein the anti-C5 antibody is administered to a patient weighing ≥40 to <60 kg at a loading dose of 2400 mg on Day 1, followed by a maintenance dose of 3000 mg on Day 15 and then once every eight weeks thereafter.

In another embodiment, a method of treating a human patient with DM is provided, wherein the anti-C5 antibody is administered to a patient weighing ≥60 to <100 kg at a loading dose of 2700 mg on Day 1, followed by a maintenance dose of 3300 mg on Day 15 and then and then once every eight weeks thereafter.

In another embodiment, a method of treating a human patient with DM is provided, wherein the anti-C5 antibody is administered to a patient weighing ≥100 kg at a loading dose of 3000 mg, followed by a maintenance dose of 3600 mg on Day 15 and then once every eight weeks thereafter.

In some embodiments, the anti-C5 antibody, or antigen binding fragment thereof, is administered alone. In other embodiments, the anti-C5 antibody, or antigen binding fragment thereof, is administered in combination with one or more additional therapeutic agents (e.g., simultaneously or separately). In one embodiment, the treatment further comprises administering Azathioprine. In another embodiment, the treatment further comprises administering Cyclosporine. In another embodiment, the treatment further comprises administering Glucocorticoid. In another embodiment, the treatment further comprises administering intramuscular glucocorticoids. In another embodiment, the treatment further comprises administering Hydroxychloroquine. In another embodiment, the treatment further comprises administering Leflunomide. In another embodiment, the treatment further comprises administering Methotrexate. In another embodiment, the treatment further comprises administering Mycophenolate mofetil/mycophenolic acid. In another embodiment, the treatment further comprises administering Sulfasalazine. In another embodiment, the treatment further comprises administering an antihistamine. In another embodiment, the treatment further comprises administering ibuprofen. In another embodiment, the treatment further comprises administering acetaminophen. In another embodiment, the treatment further comprises administering anti-pruritics. In another embodiment, the treatment further comprises administering a topical steroid. In another embodiment, the treatment further comprises administering vitamin B12. In another embodiment, the treatment further comprises administering vitamin E. In another embodiment, the treatment further comprises administering creatine. In another embodiment, the treatment further comprises administering coenzyme Q10. In another embodiment, the treatment further comprises administering a biotin supplement.

In one embodiment, the patient has not previously taken or is not taking any of the following upon initiating or during treatment: intravenous immunoglobulin (IVIg), subcutaneous immunoglobulin (SCIg), IV glucocorticoids, corticotropin injection, cyclophosphamide, rituximab, infliximab, adalimumab, etanercept, tofacitinib, ruxolitinib, or anakinra.

In one embodiment, the patient has not previously been treated with eculizumab. In another embodiment, the patient has previously been treated with eculizumab.

In another embodiment, the patient has an MMT-8 of ≤142/150 and two or more of the following prior to treatment:
a) a Patient Global Activity assessment of ≥2.0 cm on a 10 cm VAS
b) a Physician Global Activity assessment of ≥2.0 cm on a 10 cm VAS
c) a HAQ disability index with ≥0.25
d) elevation of at least one muscle enzyme at ≥1.3 times the upper limit of normal (ULN); and/or
e) a global extramuscular disease activity score with ≥2.0 cm on a 10 cm visual analog scale (VAS).

In one embodiment, the global extramuscular disease activity score is based on assessments of activity scores on the constitutional, cutaneous, skeletal, gastrointestinal, pulmonary, and cardiac scales of the MDAAT.

In another embodiment, the patient has one or more of the following prior to treatment:
a) muscle or skin biopsy with evidence of active pathological findings of DM within last 6 months prior to or upon initiating treatment;
b) electromyography evidence of active myositis within the last 6 months prior to or upon initiating treatment;
c) magnetic resonance imaging (MRI) muscle evidence of active myositis within the last 6 months prior to or upon initiating treatment;
d) at least one muscle enzyme (e.g., CK, aldolase, LDH, ALT, and/or AST) in an IMACS panel ≥2 times ULN prior to or upon initiating treatment; and/or
e) active DM skin rash characterized by inflammatory changes (CDASI Activity Score ≥7) prior to or upon initiating treatment.

In some embodiments, the disclosure relates to a composition, e.g., pharmaceutical composition or a medicament, comprising an effective amount of an anti-C5 antibody or an antigen binding fragment thereof, comprising heavy chain complementarity determining regions (HCDRs) comprising HCDR1, HCDR2 and HCDR3 sequences as set forth in SEQ ID NOs:19, 18 and 3, respectively, and light chain complementarity determining regions (LCDRs) comprising LCDR1, LCDR2 and LCDR3 sequences as set forth in SEQ ID NOs:4, and 6, respectively, for use in the treatment of DM in a human patient, wherein the composition optionally comprises background therapy for treating DM. Specifically, provided herein are compositions comprising effective amounts of ravulizumab (ULTOMIRIS®) or the antigen-binding fragment thereof, for treatment of DM in a human patient. In some embodiments, the effective amount comprises use of the dosages and scheduling described herein for the anti-C5 antibody, e.g., ravulizumab.

In further embodiments, the disclosure relates to use of an effective amount of an anti-C5 antibody, or antigen binding fragment thereof, comprising heavy chain complementarity determining regions (HCDRs) comprising HCDR1, HCDR2 and HCDR3 sequences as set forth in SEQ ID NOs:19, 18 and 3, respectively, and light chain complementarity determining regions (LCDRs) comprising LCDR1, LCDR2 and LCDR3 sequences as set forth in SEQ ID NOs:4, 5 and 6, respectively, in the manufacture of a composition, e.g., pharmaceutical composition or a medicament, for treating DM in a human patient, wherein the composition optionally comprises background therapy for the treatment of DM. Specifically, provided herein are use of an effective amount of ravulizumab (ULTOMIRIS®) or the antigen-binding fragment thereof, in the manufacture of a composition, e.g., pharmaceutical composition or a medicament, for treating DM in a human patient. In some embodiments, the effective amount comprises use of the dosages and scheduling described herein for the anti-C5 antibody, e.g., ravulizumab, optionally together with dosages and scheduling of the background therapy.

### V. Outcomes

Provided herein are methods for treating DM in a patient comprising administering to the patient an anti-C5 antibody.

Symptoms of DM include, but are not limited to, a rash (e.g., a violet-colored or dusky red rash develops, most commonly on the face, eyelids, knuckles, elbows, knees, chest and back) and/or muscle weakness (e.g., progressive muscle weakness in the muscles closest to the trunk, such as those in the hips, thighs, shoulders, upper arms and neck).

In one embodiment, patients treated according to the disclosed methods maintain a serum trough concentration of the anti-C5 antibody, or antigen binding fragment thereof, of at least 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395 or 400 μg/mL or greater. In one embodiment, patients treated according to the disclosed methods maintain a serum trough concentration of the anti-C5 antibody, or antigen binding fragment thereof, of at least 175 μg/mL or greater.

In one embodiment, patients treated according to the disclosed methods have a free C5 concentration of 0.5 μg/mL or less (e.g., 0.4 μg/mL, 0.3 μg/mL, 0.2 μg/mL, or 0.1 μg/mL or less).

The efficacy of the treatment methods provided herein can be assessed using any suitable means. In one embodiment, the treatment results in a shift towards normal levels of soluble C5b-9.

In another embodiment, the treatment results in a shift towards normal levels of myositis-specific autoantibodies (e.g., anti-melanoma differentiation-associated protein 5 antibodies (anti-MDA5 antibodies), anti-nuclear matrix protein 2 antibodies (anti-NXP2/MJ antibodies), and/or anti-synthetase/Jo 1 antibodies).

In another embodiment, the treatment results in a shift towards normal levels of muscle enzymes (e.g., creatine kinase (CK), aldolase, lactate dehydrogenase (LDH), alanine aminotransferase (ALT), and aspartate aminotransferase (AST)).

In another embodiment, the treatment results in an improvement in the patient as assessed by a International Myositis Assessment and Clinical Studies Total Improvement Scale (IMACS-TIS), compared to baseline. In one embodiment, the treatment results in an at least ≥20, 25, 30, 35, 40, 45, 50, 55, or 60-point Total Improvement Score (TIS) as assessed by an IMACS-TIS score, compared to baseline.

In another embodiment, the treatment results in an improvement in the patient as assessed by a Cutaneous Dermatomyositis Disease Area and Severity Index (CDASI), compared to baseline.

In another embodiment, the treatment results in an ≥7, 8, 9, 10, 11, or 12-point improvement in the patient as assessed by a CDASI, compared to baseline.

In another embodiment, the treatment results in an improvement in the patient as assessed by a European Quality of Life Health 5-item questionnaire dimensions 5 level (EQ-5D-5L) score, compared to baseline.

In another embodiment, the treatment results in an improvement in the patient as assessed by a Patient-reported Outcomes Measurement Information System (PROMIS), compared to baseline.

In another embodiment, the treatment results in an improvement in the patient as assessed by a Short Form Health Survey (36 questions version) (SF 36), compared to baseline.

In another embodiment, the treatment results in an improvement in the patient as assessed by a Dermatomyositis Disease Symptom Questionnaire (DM-DSQ), compared to baseline.

In another embodiment, the treatment results in an improvement in the patient as assessed by a 30-Second Chair Stand Test (30s CST), compared to baseline.

In another embodiment, the treatment results in a decrease in the patient's detectable rash as assessed by photographic analysis, compared to baseline. In one embodiment, the treatment results in a 25%, 30%, 40%, 45%, 50%, 55%, 60%, 65%, 70%, 75%, 80%, 85%, 90%, 95%, 98%, 99%, or 100% decrease in the patient's detectable rash as assessed by photographic analysis, compared to baseline.

In another embodiment, the treatment results in an improvement in the patient as assessed by a 5D-itch scale, compared to baseline.

In another embodiment, the treatment results in an improvement in the patient as assessed by a handheld dynamometry performance analysis, compared to baseline.

In another embodiment, the treatment results in an improvement in the patient as assessed by a Functional Assessment of Chronic Therapy (FACIT)-Fatigue score, compared to baseline.

In another embodiment, the treatment results in an improvement in the patient as assessed by a manual muscle testing subset of 8 muscles (MMT8), compared to baseline.

In another embodiment, the treatment results in an improvement in the patient as assessed by a Myositis Disease Activity Assessment Tool (MDAAT), compared to baseline.

In another embodiment, the treatment results in an improvement in the patient as assessed by a Physician Global Activity Assessment, compared to baseline.

In another embodiment, the treatment results in an improvement in the patient as assessed by a Patient Global Activity Assessment, compared to baseline.

In another embodiment, the treatment results in an improvement in the patient as assessed by a Health Assessment Questionnaire (HAQ), compared to baseline.

In another embodiment, the treatment results in an improvement in the patient as assessed by Cutaneous Dermatomyositis Activity Physician's Global Assessment (CD-IGA), compared to baseline.

In another embodiment, the treatment results in a reduction or cessation in the patient's rash and/or muscle weakness.

In another embodiment, the treatment results in terminal complement inhibition.

In another embodiment, the treatment results in a reduction in adverse events.

### VI. Kits and Unit Dosage Forms

Also provided herein are kits that include a pharmaceutical composition containing an anti-C5 antibody or antigen binding fragment thereof, such as ravulizumab or BNJ421, and a pharmaceutically acceptable carrier, in a therapeutically effective amount adapted for use in the preceding methods. The kits optionally also can include instructions, e.g., comprising administration schedules, to allow a practitioner (e.g., a physician, nurse, or patient) to administer the composition contained therein to administer the composition to a patient having DM. The kit also can include a syringe.

Optionally, the kits include multiple packages of the single-dose pharmaceutical compositions each containing an effective amount of the anti-C5 antibody, or antigen binding fragment thereof, for a single administration in accordance with the methods provided above. Instruments or devices necessary for administering the pharmaceutical composition(s) also may be included in the kits. For instance, a kit may provide one or more pre-filled syringes containing an amount of the anti-C5 antibody or antigen binding fragment thereof.

In one embodiment, a kit for treating DM in a human patient comprises: (a) a dose of an anti-C5 antibody or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and (b) instructions for using the anti-C5 antibody, or antigen binding fragment thereof, according to any of the methods described herein. In one embodiment, the anti-C5 antibody, or antigen binding fragment thereof, is administered:
(a) at a loading dose of 900 mg, followed by a maintenance dose of 2100 mg two weeks later and then once every eight weeks thereafter to a patient weighing <30 kg;
(b) at a loading dose of 1200 mg, followed by a maintenance dose of 2700 mg two weeks later and then once every eight weeks thereafter to a patient weighing ≥30 to <40 kg;
(c) at a loading dose of 2400 mg, followed by a maintenance dose of 3000 mg two weeks later and then once every eight weeks thereafter to a patient weighing ≥40 to <60 kg;
(d) at a loading dose of 2700 mg, followed by a maintenance dose of 3300 mg two weeks later and then once every eight weeks thereafter to a patient weighing ≥60 to <100 kg; or
(e) at a loading dose of 3000 mg, followed by a maintenance dose of 3600 mg two weeks later and then once every eight weeks thereafter to a patient weighing ≥100.

The following examples are merely illustrative and should not be construed as limiting the scope of this disclosure in any way as many variations and equivalents will become apparent to those skilled in the art upon reading the present disclosure. The contents of all references, Genbank entries, patents and published patent applications cited throughout this application are expressly incorporated herein by reference.

### Example

### Example 1: A Phase 2/3, Double-Blind, Randomized, Placebo-Controlled, Parallel Group, Multicenter Study to Evaluate the Efficacy and Safety of Ravulizumab in Adult Participants with Dermatomyositis

### 1. Objectives

a. Phase 2 Objectives

The primary objective of the study is to determine the effect of ravulizumab compared with placebo in the treatment of DM based on improvement in Total Improvement Score (TIS) as per 2016 ACR/EULAR Myositis Response Criteria for adult DM (also known as IMACS) (e.g., as assessed by proportion of participants with a ≥20-point improvement response on IMACS TIS (TIS20) at Week 26 of the Randomized Controlled Period).

A secondary objective is to assess the efficacy of ravulizumab compared with placebo in the treatment of DM based on improvement in efficacy endpoints (e.g., based on (a) mean TIS at Week 26, (b) mean change from baseline in CDASI Activity Score at Week 26, (c) change from baseline of each of the IMACS core set measures at Week 26, (d) time to first CDASI Activity Score improvement (minimally clinically important differences [MCID]=7-point improvement), (e) proportion of participants with CDASI MCID improvement at Week 26, (f) change in CD-IGAat Week 26, (g) proportion of participants with TIS20 at each visit, (h) proportion of participants with TIS40 at each visit, (i) proportion of participants with TIS60 at each visit, (j) time to First Response of TIS20, TIS40, or TIS60 respectively, and/or (k) time to first IMACS myositis core set measure improvements (see MCID for each core set measure).

A further objective is to characterize the PK/PD and immunogenicity of ravulizumab in adult participants with DM (e.g., as assessed by serum ravulizumab concentration over the study duration, change in serum free and total C5 concentrations over the study duration, and presence and titer of ADAs over the study duration).

Another objective is to characterize the overall safety of ravulizumab in participants with DM (e.g., via incidence of treatment-emergent adverse events (TEAEs), treatment-emergent serious adverse events (TESAEs), and TEAEs leading to study intervention discontinuation).

Exploratory objectives include evaluating (1) the effect of ravulizumab on overall health-related quality of life (HR-QoL) and participant-centered and participant-reported outcomes in DM (e.g., as assessed by change from baseline in EQ 5D 5L at each visit, change from baseline in PROMIS 29 v2.1 domains over time, change from baseline in SF-36 over time, change from baseline of DM symptoms captured in DM-DSQ over time, and change from baseline of Patient Self-Assessment of Disease Activity (last question in the DM-DSQ) over time), (2) complement, inflammatory, autoimmune, and other soluble biomarkers in adult participants with DM (e.g., as assessed by presence of myositis-specific autoantibodies (e.g., anti MDA5, anti-NXP2/MJ, and anti-synthetase/Jo-1) in blood and change from baseline in specific autoantibody/autoantibodies titer over the course of the study) and change from baseline in plasma complement activation (e.g., sC5b-9, etc.) over the course of the study), (3) change in visible clinical skin manifestations in participants with DM concurrently receiving ravulizumab (e.g., as assessed by change in area with detectable rash using a specialized photographic analysis at each visit), (4) the efficacy of ravulizumab in the treatment of DM based on other efficacy endpoints throughout the study (e.g., as assessed by change from baseline using scale to measure pruritus (5D-itch scale) at each visit, proportion of participants meeting Clinical Worsening criteria, change from baseline in handheld dynamometry performance at each visit, change from baseline in 30-second Chair Stand Test (30s CST) at each visit, and change from baseline in FACIT-Fatigue score over time).

b. Phase 3 Objectives

The primary Phase 3 objective is to determine the effect of ravulizumab compared with placebo in the treatment of DM based on improvement in TIS as per 2016 IMACS (e.g., as assessed by proportion of participants with a≥20-point improvement response on IMACS-TIS (TIS20) at Week 50 as per defined composite estimand of the Randomized Controlled Period).

A key secondary objective is to assess the efficacy of ravulizumab compared with placebo in the treatment of DM based on improvement in components of IMACS and other myositis activity measures (e.g., as assessed by mean TIS at Week 50, mean change from baseline in MMT-8 at Week 50, mean change from baseline in extra-muscular disease activity based on MDAAT at Week 50, and mean change from baseline in CDASI Activity Score at Week 50).

The secondary objective is to assess the efficacy of ravulizumab compared with placebo in the treatment of DM based on other efficacy endpoints (e.g., as assessed by (a) mean change from baseline in patient global activity assessment at Week 50, (b) mean change from baseline in physician global activity assessment at Week 50, (c) mean change from baseline in HAQ at Week 50, (d) mean change from baseline in muscle enzyme values at Week 50, (e) mean TIS at each visit from Week 2 through Week 50, (f) proportion of participants with TIS20 at each visit, (g) proportion of participants with TIS40 at each visit, (h) proportion of participants with TIS60 at each visit, (i) time to First Response of TIS20, TIS40, or TIS60, respectively, (j) time to first IMACS myositis core set measure improvements (see MCID for each core set measure), (k) T=time to first CDASI Activity Score improvement (minimally clinically important differences [MCID]=7-point improvement), (1) proportion of participants with CDASI MCID improvement at Week 50, and (m) change in CD-IGA at Week 50).

A further objective is to characterize the PK/PD and immunogenicity of ravulizumab in adult participants with DM (e.g., as assessed by serum ravulizumab concentration over the study duration, change in serum free and total C5 concentrations over the study duration, and presence and titer of ADAs over the study duration).

Another objective is to characterize the overall safety of ravulizumab in participants with DM (e.g., as assessed by incidence of treatment-emergent adverse events (TEAEs), treatment-emergent serious adverse events (TESAEs), and TEAEs leading to study intervention discontinuation).

Exploratory objectives include evaluating: (1) the effect of ravulizumab compared with placebo on overall health-related quality of life (HR-QoL) and participant centered and participant reported outcomes in DM (e.g., as assessed by change from baseline in EQ 5D 5L at each visit, change from baseline in PROMIS 29 v2.1 domains over time, change from baseline in SF 36 over time, change from baseline of DM symptoms captured in DM-DSQ over time, and change from baseline of Patient Self-Assessment of Disease Activity (last question in the DM-DSQ) over time), (2) complement, inflammatory, autoimmune, and other soluble biomarkers in adult participants with DM (e.g., as assessed by presence of myositis-specific autoantibodies (e.g., anti MDAS, anti-NXP2/MJ, and anti-synthetase/Jo 1) in blood, and change from baseline in specific autoantibody/autoantibodies titer over the course of the study and change from baseline in plasma complement activation (e.g., sC5b-9, etc) over the course of the study, (3) change in visible clinical skin manifestations in participants with DM concurrently receiving ravulizumab (e.g., as assessed by change in area with detectable rash using a specialized photographic analysis), (4) the efficacy of ravulizumab compared with placebo in the treatment of DM based on other efficacy endpoints throughout the study (e.g., as assessed by change from baseline using scale to measure pruritus (5D-itch scale) at each visit, proportion of participants meeting disease worsening criteria, change from baseline in handheld dynamometry performance at each visit, change from baseline in 30-second Chair Stand Test (30s CST) at each visit, and change from baseline in FACIT-Fatigue score over time).

c. Open Label Extension Objectives

Objectives for the open label extension study include assessing: (1) long-term efficacy of ravulizumab in the treatment of DM based on other efficacy endpoints throughout the study (e.g., via change from baseline using scale to measure pruritus (5D-itch scale) at each visit, change from baseline in CDASI Activity Score at each visit, change in CD-IGA at Week 78, proportion of participants meeting disease worsening criteria, change from baseline in handheld dynamometry performance at each visit, change from baseline in 30-second Chair Stand Test (30s CST) at each visit, and change from baseline in FACIT-Fatigue score at each visit), and the long-term use of DM treatment in participants with DM concurrently receiving ravulizumab (e.g., via percentage of participants steroid free or minimal maintenance dose of 5 mg or less at the end of the OLE Period, cumulative steroid dose from baseline, number of rescue therapy events and time to first rescue therapy treatment, and percentage of participants who changed allowed DM treatment and time to first DM treatment change).

Exploratory objectives include: (1) evaluating the long-term effect of ravulizumab treatment in participants with DM based on improvement in components of the IMACS (e.g., via mean TIS at Week 78, change from baseline in MMT-8 at each visit, change from baseline in physician global activity assessment at each visit, change from baseline in extra-muscular disease activity based on MDAAT at each visit, and change from baseline in HAQ at each visit), (2) evaluating the long-term effect of ravulizumab on overall health-related quality of life (HR-QoL) and participant-centered and participant-reported outcomes in DM (e.g., via change from baseline in EQ-5D-5L at each visit, change from baseline in PROMIS 29 v2.1 domains at each visit, change from baseline in SF-36 at each visit, change from baseline of DM symptoms captured in DM-DSQ at each visit, and change from baseline of Patient Global Assessment of Disease Activity (last question in the DM-DSQ) at each visit), (3) characterizing the long-term safety of ravulizumab in participants with DM (e.g., via incidence of treatment-emergent adverse events (TEAEs), treatment-emergent serious adverse events (TESAEs), and TEAEs leading to study intervention discontinuation), (4) characterizing the long-term PK/PD and immunogenicity of ravulizumab in adult participants with DM (e.g., via serum ravulizumab concentration over the study duration, change in serum free and total C5 concentrations over the study duration, and presence and titer of ADAs over the study duration), and (5) evaluating complement, inflammatory, autoimmune and other soluble biomarkers in adult participants with DM (e.g., via change from baseline (only in subjects positive at baseline) in specific autoantibody (e.g., anti-MDA5, anti-NXP2/MJ, and anti-synthetase/Jo-1) titers over the course of the study and change from baseline in plasma complement activation (e.g., sC5b-9, etc) over the course of the study).

### 2. Primary Estimands

Part A of the study is exploratory in nature and no primary estimand is predefined.

For Part B, the primary estimand is the difference in the proportion of response (TIS20) between ravulizumab and placebo at the end of the Randomized Controlled Period in participants with DM as defined by the study entry criteria. The primary estimand is based on a composite strategy based on the following.

Participants who have any of the following intercurrent events are considered as treatment failures for the purposes of the primary estimand during the Randomized Controlled Period: (1) discontinued the study prematurely due to safety, tolerability, or efficacy reasons related to ravulizumab, (2) study intervention for safety, tolerability, or efficacy concerns during the Randomized Controlled Period, (3) increased dose or initiated new DM treatments (glucocorticoid and/or ISTs) during the Randomized Controlled Period for any reason, or (4) taken prohibited medications/therapies at any point during the Randomized Controlled Period.

All participants without any intercurrent event have their IMACS-TIS assessments used for the purposes of the primary estimand.

Anyone who has discontinued study intervention but not withdrawn consent is permitted to remain in the study to complete scheduled assessments.

Participants who have any of the following intercurrent events are considered as treatment failures for the purposes of the primary estimand during the Randomized Controlled Period: discontinued the study prematurely due to safety, tolerability, or efficacy reasons related to ravulizumab.

### 3. Study Design

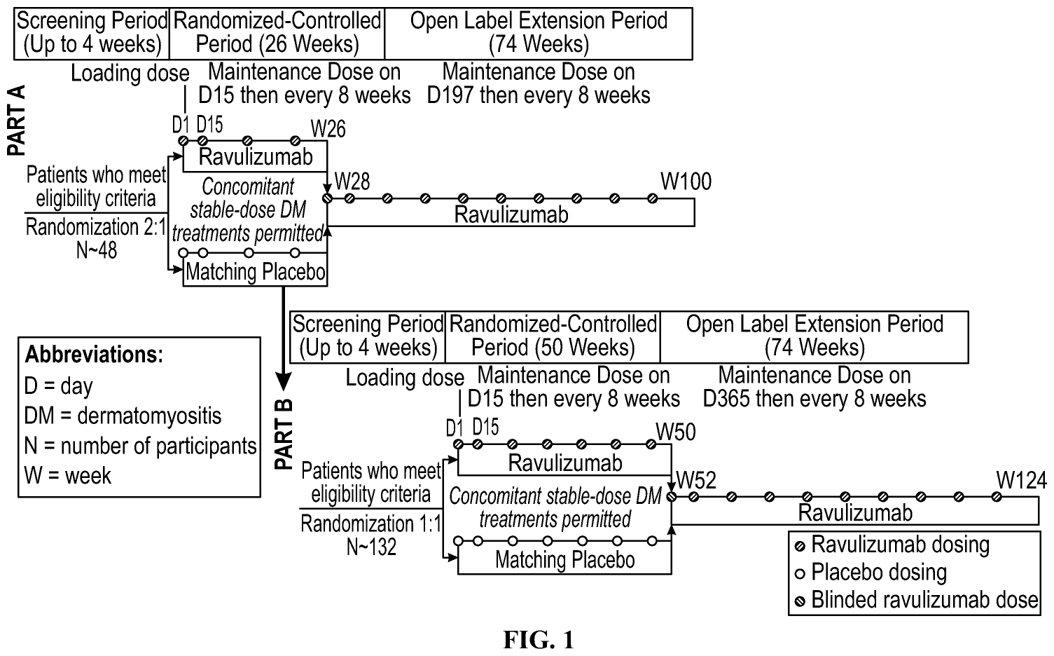

Study ALXN1210-DM-310 is a Phase 2/3, double-blind, randomized, placebo-controlled, parallel group, multicenter study to evaluate the efficacy, safety, PK, PD, and immunogenicity of ravulizumab in adult participants with DM. An overview of the study is set forth in FIG. 1. The study is conducted in 2 parts: Part A (Phase 2) and Part B (Phase 3). For the purposes of this protocol, information provided that is applicable to only Part A or Part B is clearly demarcated. Information without a specification as to whether it pertains to either Part A or Part B is applicable to both parts.

a. Part A (Phase 2 Portion)

There are 3 periods in Part A of this study: Screening Period, Randomized Controlled Period, and OLE Period. Participants are screened for eligibility for up to 4 weeks during the Screening Period. Approximately 48 eligible participants are randomized in a 2:1 ratio to receive either weight-based IV infusion of ravulizumab or matching placebo during the double-blind Randomized Controlled Period. Upon completing the last assessment of the Randomized Controlled Period at Week 26, participants can continue into the OLE Period.

For each participant, the Randomized Controlled Period ends and the OLE Period begins at Week 26. During the OLE Period, participants in the ravulizumab group continue to receive ravulizumab treatment, and participants in the placebo group switch to receive ravulizumab treatment. Participants receive ravulizumab until ravulizumab is registered or approved (in accordance with country specific regulations) or for up to 74 weeks (approximately 1.5 years), whichever occurs first.

Participants in the Randomized Controlled Period who discontinue study intervention early for any reason and agree to remain in the study are followed up for safety and efficacy assessments for the duration of the Randomized Controlled Period. If the decision to discontinue study intervention falls outside of a study visit, participants are expected to return to an unscheduled visit as soon as possible within a week. Participants in the Randomized Controlled Period who discontinue study intervention early and do not agree to continue in the study are expected to complete the study visit (if at the site for a scheduled visit) or return for an unscheduled visit as soon as possible within a week and an ET Visit 8 weeks after the participant's last dose of study intervention. In addition, a follow-up phone call 20 weeks after the participant's last dose of study intervention is performed to collect concomitant medications, non-pharmacologic therapies and procedures, and AEs. Participants who discontinue study intervention during the Randomized Controlled Period are not eligible for the OLE Period.

Eight weeks after the final dose of study intervention is administered in the OLE Period, all enrolled participants return for an EOS Visit at Week 100, during which final study assessments are conducted. Participants in the OLE Period who discontinue the study intervention early are expected to complete the study visit (if at the site for a scheduled visit) or return as soon as possible within a week for an unscheduled visit and an ET visit 8 weeks after the participant's last dose of study intervention. In addition, a follow-up phone call 20 weeks after the participant's last dose of study intervention is performed to collect concomitant medications, non-pharmacologic therapies and procedures, and AEs.

Attempts should be made to follow up with all participants for safety for 8 weeks after their last dose of study intervention.

One interim analysis planned for Part A for administrative purposes. The interim analysis is conducted with discretion when approximately 50% of participants have completed the pre dose Week 26 visit or discontinued prematurely from the Randomized Controlled Period of Part A. An independent statistical is established to conduct the unblinded interim analysis.

Participants from Part A are not enrolled in Part B of the study (Phase 3), and therefore, do not contribute to the formal statistical hypothesis testing of Part B.

b. Part B (Phase 3 Portion)

There are 3 periods in Part B of this study: Screening Period, Randomized Controlled Period, and OLE Period. Approximately 132 eligible participants are stratified by region (North America, Europe, and Asia-Pacific) and cutaneous manifestations (Cutaneous Dermatomyositis Disease Area and Severity Index [CDASI] Activity Score <14 and >14) and randomized 1:1 either to ravulizumab IV infusion or placebo IV infusion.

For each participant, the Randomized Controlled Period ends and the OLE Period begins at Week 50. During the OLE Period, participants in the ravulizumab group continue to receive ravulizumab, and participants in the placebo group switch to receive ravulizumab. Participants receive ravulizumab until ravulizumab is registered or approved (in accordance with country specific regulations) or for up to 74 weeks (approximately 1.5 years), whichever occurs first.

Participants in the Randomized Controlled Period who discontinue study intervention early and agree to remain in the study are followed up for safety and efficacy assessments for the duration of the Randomized Controlled Period. If the decision to discontinue study intervention falls outside of a study visit, participants are expected to return to an unscheduled visit as soon as possible within a week.

Participants in the Randomized Controlled Period who discontinue study intervention early and do not agree to continue in the study, are expected to complete the study visit (if at the site for a scheduled visit) or return for an unscheduled visit as soon as possible within a week and an ET visit 8 weeks after the participant's last dose of study intervention. In addition, a follow-up phone call 20 weeks after the participant's last dose of study intervention is performed to collect concomitant medications, non-pharmacologic therapies and procedures, and adverse events (AEs). Participants who discontinue study intervention during the Randomized Controlled Period are not eligible for the OLE period. Eight weeks after the final dose of study intervention is administered in the OLE Period, all enrolled participants return for an EOS Visit at Week 124, during which final study assessments are conducted. Participants in the OLE Period who discontinue the study intervention early are expected to complete the study visit (if at the site for a scheduled visit) or return as soon as possible within a week for an unscheduled visit and an ET visit 8 weeks after the participant's last dose of study intervention. In addition, a follow-up phone call 20 weeks after the participant's last dose of study intervention is performed to collect concomitant medications, non-pharmacologic therapies and procedures, and AEs.

Attempts are made to follow up with all participants for safety for 8 weeks after their last dose of study intervention.

One interim analysis is planned for sample-size re-estimation for Part B. The unblinded interim analysis is performed by an independent Data Monitoring Committee (DMC). Treatment allocation during the Randomized Controlled Period remains blinded throughout the study. The interim analysis for sample size re estimation is conducted with discretion when approximately 30% of participants have completed the pre-dose Week 50 visit or discontinued prematurely from Part B.

c. Clinical Worsening

For this protocol, Clinical Worsening is defined as exhibiting one of the following criteria on 2 consecutive visits approximately 4 weeks apart:
a. Physician's global activity visual analog scale (VAS) worsening ≥2 cm and manual muscle testing subset of 8 muscles (MMT-8) worsening ≥20% compared to baseline;
b. Global extra muscular activity worsening ≥2 cm on the Myositis Disease Activity Assessment Tool (MDAAT) VAS compared to baseline; and/or
c. Any 3 of 5 core set measures (CSM) (excluding muscle enzymes) worsening by ≥30% compared to baseline.

Worsening is determined using the criteria provided above. An Unscheduled Visit is held 4 weeks after the visit during which a participant shows the first signs of worsening. At the Unscheduled Visit, the Investigator checks the criteria above to determine whether the participant continues to show signs of worsening. If a participant meets the definition of Clinical Worsening, glucocorticoid rescue therapy may be administered.

The participant may receive rescue medication that is an increased dose of a medication currently used to treat their DM symptoms or a newly initiated DM treatment (glucocorticoid and/or ISTs) and they are discontinued from study intervention.

Participants in the Randomized Controlled Period who discontinue study intervention early for any reason and agree to remain in the study are followed up for safety and efficacy assessments for the duration of the Randomized Controlled Period. If the decision to discontinue study intervention falls outside of a study visit, participants are expected to return to an unscheduled visit as soon as possible within a week.

Participants in the Randomized Controlled Period who discontinue study intervention early and do not agree to continue in the study, are expected to complete the study visit (if at the site for a scheduled visit) or return for an unscheduled visit as soon as possible within a week and an ET Visit 8 weeks after the participant's last dose of study intervention. In addition, a follow-up phone call 20 weeks after the participant's last dose of study intervention is performed to collect concomitant medications, non-pharmacologic therapies and procedures, and AEs. The Investigator determines whether a participant who meets the definition of Clinical Worsening should remain on study intervention. Participants who discontinue study intervention during the Randomized Controlled Period are not eligible for the OLE Period.

Clinical Worsening criteria is checked at the visits during both the Randomized Controlled Period and the OLE Period.

d. Completion of Treatment

Participants are considered to have completed the study treatment in the Randomized Controlled Period if they do not permanently discontinue study intervention before the Week 26 (Part A) or Week 50 (Part B) Visit. All participants who discontinue the study intervention during the Randomized Controlled Period are followed up for safety and efficacy assessments until the end of the Randomized Controlled Period, withdrawal of consent, or lost to follow-up, whichever occurs earlier.

Participants are considered to have completed the study treatment in the OLE Period if they do not discontinue treatment with ravulizumab before the Week 100 (Part A) or Week 24 (Part B) EOS Visit, the participant terminates the study early due to termination of the study, or the participant transitions to approved product.

Participants are considered to have completed the Randomized Controlled Period of the study if they complete the last scheduled visit specified in the Schedule of Activities for this period.

### 4. Study Population

a. Inclusion Criteria

Participants are eligible to be included in the study only if all of the following criteria apply:
1. 18 years of age or older at the time of signing the informed consent.
2. Body weight ≥30 kg at the time of Screening.
3. Male or female.
4. Diagnosis: Meet 2017 ACR/EULAR classification criteria for definite or probable DM (see, Lundberg I E, et al., Arthritis Rheumatol. 2017; 69(12):2271-2282).
5. Participants who have an inadequate response or are intolerant to 2 or more DM treatments, including systemic glucocorticoids or ISTs (e.g., azathioprine, methotrexate, rituximab, IVIg), either in combination or as monotherapy.
6. Participants under treatment with glucocorticoids and/or ISTs who are on stable therapy as described in Table 5 and who are able to remain on stable therapy throughout the Randomized Controlled Period OR participants under treatment with glucocorticoids and/or ISTs who complete an appropriate washout period for these treatments before the Day 1 Visit as described in Table 12 (e.g., 6-month washout period for rituximab; 3-month washout period for IVIg or subcutaneous immunoglobulin [SCIg]).
7. Disease severity: Participants who show disease severity at Screening using the following criteria: MMT-8 of ≤142/(out of a maximum of 150) and 2 additional IMACS CSMs out of 5 at Screening such as: (a) Patient Global Activity assessment of ≥2.0 cm on a 10 cm VAS, (b) Physician Global Activity assessment of ≥2.0 cm on a 10 cm VAS, (c) HAQ disability index with ≥0.25, (d) elevation of at least one of the muscle enzymes (which includes creatine kinase [CK], aldolase, lactate dehydrogenase [LDH], alanine aminotransferase [ALT], and aspartate aminotransferase [AST]) at ≥1.3 times the upper limit of normal (ULN), and/or (e) Global extramuscular disease activity score with ≥2.0 cm on a 10 cm VAS (this measure is the physician's composite evaluation and is based on assessments of activity scores on the constitutional, cutaneous, skeletal, gastrointestinal, pulmonary, and cardiac scales of the MDAAT).
8. Disease activity: Participants must meet one of the following criteria to ensure enrollment of active disease rather than weakness due to muscle damage. An Independent Adjudication Committee (IAC) is used to assess eligibility for participants who do not meet the criteria listed below but who in the opinion of the Investigator qualify for the study.
   a. Muscle or skin biopsy with evidence of active pathological findings of DM within the last 6 months prior to or at Screening;
   b. Electromyography evidence of active myositis within the last 6 months prior to Screening;
   c. Magnetic resonance imaging (MRI) muscle evidence of active myositis within the last 6 months prior to Screening;
   d. At least 1 muscle enzyme (CK, LDH, aldolase, AST, ALT) in the IMACS panel ≥2 times ULN at Screening; and/or
   e. Active DM skin rash characterized by inflammatory changes (CDASI Activity Score ≥7) at Screening.
9. Vaccinated against N meningitidis within 3 years prior to initiating ravulizumab as per national and local guidelines. Participants must receive the vaccination at least 2 weeks before first study intervention. National and local guidelines for prophylactic antibiotics are also followed.
10. Participants who have been diagnosed with cancer within the last 3 years need to have appropriate negative cancer screening as per local standard of care within 6 months before Screening (basal or squamous cell skin cancer or carcinoma in situ of the cervix needs to have been excised and cured at least 3 months before Screening).
11. Female participants of childbearing potential and male participants must follow specified contraception guidance as described in the protocol.
12. Capable of giving signed informed consent as described in the protocol, which includes compliance with the requirements and restrictions listed in the informed consent form (ICF) and the protocol.
   b. Exclusion Criteria Participants are excluded from the study if any of the following criteria apply
1. Cancer-associated myositis, defined as the diagnosis of myositis within 3 years of the diagnosis of cancer (except basal or squamous cell skin cancer or carcinoma in situ of the cervix that has been excised and cured and at least 3 months before Screening).
2. Evidence of active malignant disease or malignancies diagnosed within the previous 5 years (including hematological malignancies and solid tumors) (except basal or squamous cell skin cancer or carcinoma in situ of the cervix that has been excised and cured for at least 3 months before Screening).
3. Participants with other forms of myositis: Clinical diagnosis of inclusion body myositis, polymyositis, necrotizing myopathy, drug induced myositis/myopathy, anti-synthetase syndrome without DM, cancer associated myositis (myositis diagnosed within 3 years either before or after a diagnosis of malignancy except squamous cell cancer of skin, basal cell cancer, carcinoma in situ lesions anywhere, or cervical carcinoma in situ), myositis with overlap connective tissue disease such as SLE, rheumatoid arthritis, or systemic sclerosis. Participants with secondary Sjogren's syndrome are allowed.
4. Participants with significant muscle damage (e.g., severe muscle atrophy, end stage muscle disease, MRI with severe atrophy or fibrofatty replacement) as per investigator opinion.
5. History of N meningitidis infection.
6. Human immunodeficiency virus (HIV) infection (evidenced by HIV Type 1 or Type 2 antibody titer).
7. History of unexplained infections.
8. Active systemic bacterial, viral, or fungal infection within 14 days prior to ravulizumab administration.
9. Presence of fever ≥38° C. (100.4° F.) within 7 days prior to study intervention administration on Day 1.
10. History of hypersensitivity to murine proteins or to 1 of the excipients of ravulizumab.
11. Participants with advanced clinically symptomatic interstitial lung disease (in the opinion of the Investigator) supported by a history of fibrosis on chest x-ray, history of interstitial lung disease on computed tomography (CT) scan of the lungs, or documented history of forced vital capacity, percent predicted <80% related to DM.
12. Any medical condition (such as, cardiac, pulmonary, renal, oncologic, psychiatric disease, or with a diagnosis of fibromyalgia) that, in the opinion of the Investigator, might interfere with the participant's participation in the trial, poses any added risk for the participant, or confounds the assessment of the participant. This includes participants with severe arthritis with limited range of motion or severe diffuse calcinosis or other condition which prevents quantitative muscle strength testing.
13. History of drug and/or alcohol abuse (according to Diagnostic and Statistical Manual of Mental Disorders [DSM]5) within 1 year of Screening Visit that would limit participant participation in the study as determined by the Investigator.
14. Participants unwilling or unable to complete an appropriate washout period for any of the prohibited medications before the Day 1 visit.
15. Received ≥1 mg/kg/day of prednisone or equivalent within 8 weeks before randomization or is currently on a prednisone or equivalent maintenance dose that is >20 mg/day.
16. Previously or currently treated with a complement inhibitor.
17. Participation in any other investigational drug study or exposure to an investigational drug or device within 30 days of Screening or 5 half-lives of the investigational drug, whichever is greater.
18. Participants who begin a new muscle strengthening exercise program for myositis (e.g., physical therapy [PT]) in the last 4 weeks or change their current exercise regimen up to 4 weeks before the Day 1 visit. Participants should not initiate any new exercise program or change their current exercise regimen throughout the randomized phase of the trial.
19. Pregnant, breastfeeding, or intending to conceive during the course of the study.
20. Inability or unwillingness to adhere to the protocol requirements.

### 5. Study Intervention

Study intervention is defined as any investigational intervention(s), marketed product(s), placebo, or medical device(s) intended to be administered to a study participant according to the study protocol.

Ravulizumab IV is formulated at 10 mg/mL and 100 mg/mL concentrations with a pH 7.0 and 7.4, respectively. Both formulations are presented as sterile, preservative-free solutions for IV administration and are supplied in single-use vials.

The placebo comparator product is formulated as a matching sterile, clear, colorless solution with the same buffer components, but without active ingredient.

Details regarding the ravulizumab IV formulation are set forth in Table 1.

TABLE 1
Study Intervention

| | | |
|---|---|---|
| Study | Ravulizumab | Placebo |
| intervention | | |
| name | | |
| Dose | Vial | Vial |
| formulation | | |
| Physical | Liquid solution practically free | Liquid solution practically free |
| description | from particles | from particles |
| Dosage Form | Concentrated sterile, preservative- | Sterile, preservative-free aqueous |
| | free aqueous solution in single-use | solution in single-use 30 mL vials |
| | 30 mL vials (10 mg/mL formulation) | |
| | or single-use 11 mL vials (100 mg/mL | |
| | formulation) | |
| Unit dose | 300 mg (10 mg/mL formulation) or | Placebo |
| strength(s) | 1100 mg (100 mg/mL formulation) | |
| Dosage level(s)^{a} | Weight-based dosing | Weight-based dosing |
| Route of | Intravenous (IV) infusion | IV infusion |
| administration | | |
| Use | Experimental | Placebo comparator |
| IMP and NIMP | Investigational Medicinal Product | IMP |
| | (IMP) | |
| Sourcing | Provided centrally by Alexion or | Provided centrally by Alexion or |
| | contracted manufacturing organization | contracted manufacturing organization |
| Packaging and | Ravulizumab is provided in glass vials | Placebo is provided in glass vials |
| labeling | and stoppered with a butyl rubber | and stoppered with a butyl rubber |
| | stopper with an aluminum overseal and | stopper with an aluminum overseal |
| | a flip-off cap. Study intervention | and a flip-off cap. Placebo is |
| | are supplied in kits and labeled as | supplied in kits and labeled as |
| | required per country requirement. | required per country requirement. |

At the scheduled dosing visits, study intervention infusion should be performed after all other tests and procedures have been completed, excluding the post-dose blood sampling for PK/PD.

The ravulizumab or placebo dose for each participant is based on body weight and is administered as an IV infusion. The dosing regimen consists of a loading dose followed by maintenance dosing administered q8w (see Table 2). The maintenance dosing is initiated 2 weeks after loading dose administration. Table 3 is a reference chart for weight-based dosing.

TABLE 2
Weight-Based Doses of Ravulizumab

| Body Weight | | |
|---|---|---|
| Range (kg)^{a, b} | Loading Dose (mg) | Maintenance Dose (mg) |
| ≥30 to <40 | 1200 | 2700 |
| ≥40 to <60 | 2400 | 3000 |
| ≥60 to <100 | 2700 | 3300 |
| ≥100 | 3000 | 3600 |

Note:
Additional dose preparation instructions are provided in the pharmacy manual.
^{a}Dose regimen is based on the last recorded study visit body weight. If the study intervention is prepared the night before a visit, the weight from the most recent study visit should be used.
^{b}In the event that a participant drops below 30 kg during the course of the study, the approved ravulizumab atypical hemolytic uremic syndrome (aHUS) dosing for patients weighing 20 to 30 kg is used: a loading dose of 900 mg and maintenance dose of 2100 mg.

TABLE 3
Reference Chart for Weight-Based Dosing

| | | | | | | | |
|---|---|---|---|---|---|---|---|
| | | | | | | Diluent (0.9% | |
| | | | | | | Sodium | |
| | | Body | Ravulizumab | Ravulizumab | Placebo | Chloride) | Total |
| Study Period | Dosing | Weight (kg)^{a} | Dose (mg) | Volume (mL) | Volume (mL) | Volume (mL) | Volume (mL) |

Ravulizumab Group

| | | | | | | | |
|---|---|---|---|---|---|---|---|
| Randomized | Loading dose | ≥30 to <40 | 1200 | 120 | 0 | 120 | 240 |
| Controlled | (Day 1) | ≥40 to <60 | 2400 | 240 | 0 | 240 | 480 |
| (10 mg/mL | | ≥60 to <100 | 2700 | 270 | 0 | 270 | 540 |
| formulation) | | ≥100 | 3000 | 300 | 0 | 300 | 600 |
| | Maintenance dose | ≥30 to <40 | 2700 | 270 | 0 | 270 | 540 |
| | (Part A Days 15, | ≥40 to <60 | 3000 | 300 | 0 | 300 | 600 |
| | 71, 127) | ≥60 to <100 | 3300 | 330 | 0 | 330 | 660 |
| | (Part B: Days 15, | ≥100 | 3600 | 360 | 0 | 360 | 720 |
| | 71, 127, 183, | | | | | | |
| | 239, 295) | | | | | | |
| Open-Label | Blinded dose | ≥30 to <40 | 900 | 90 | 30 | 120 | 240 |
| Extension | (Part A: Day 183) | ≥40 to <60 | 900 | 90 | 150 | 240 | 480 |
| (10 mg/mL | (Part B: Day 351) | ≥60 to <100 | 900 | 90 | 180 | 270 | 540 |
| formulation) | | ≥100 | 900 | 90 | 210 | 300 | 600 |
| | Open-label | ≥30 to <40 | 2700 | 270 | 0 | 270 | 540 |
| | maintenance dose | ≥40 to <60 | 3000 | 300 | 0 | 300 | 600 |
| | (Part A: Days 197 | ≥60 to <100 | 3300 | 330 | 0 | 330 | 660 |
| | to 645, every 8 | ≥100 | 3600 | 360 | 0 | 360 | 720 |
| | weeks [q8w]) | | | | | | |
| | (Part B: Days | | | | | | |
| | 365 to 813, q8w) | | | | | | |
| Open-Label | Open-label | ≥30 to <40 | 2700 | 27 | 0 | 27 | 54 |
| Extension | maintenance dose | ≥40 to <60 | 3000 | 30 | 0 | 30 | 60 |
| (100 mg/mL | (Part A: Days 197 | ≥60 to <100 | 3300 | 33 | 0 | 33 | 66 |
| Formulation) | to 645, q8w) | ≥100 | 3600 | 36 | 0 | 36 | 72 |
| | (Part B: Days 365 | | | | | | |
| | to 813, q8w) | | | | | | |

Placebo Group

| | | | | | | | |
|---|---|---|---|---|---|---|---|
| Randomized | Loading dose | ≥30 to <40 | 0 | 0 | 120 | 120 | 240 |
| Controlled | (Day 1) | ≥40 to <60 | 0 | 0 | 240 | 240 | 480 |
| (10 mg/mL | | ≥60 to <100 | 0 | 0 | 270 | 270 | 540 |
| formulation) | | ≥100 | 0 | 0 | 300 | 300 | 600 |
| | Maintenance dose | ≥30 to <40 | 0 | 0 | 270 | 270 | 540 |
| | (Part A: Days 15, | ≥40 to <60 | 0 | 0 | 300 | 300 | 600 |
| | 71, 127) | ≥60 to <100 | 0 | 0 | 330 | 330 | 660 |
| | (Part B: Days 15, | ≥100 | 0 | 0 | 360 | 360 | 720 |
| | 71, 127, 183, | | | | | | |
| | 239, 295) | | | | | | |
| Open-Label | Blinded loading | ≥30 to <40 | 1200 | 120 | 0 | 120 | 240 |
| Extension | dose | ≥40 to <60 | 2400 | 240 | 0 | 240 | 480 |
| (10 mg/mL | (Part A: Day 183) | ≥60 to <100 | 2700 | 270 | 0 | 270 | 540 |
| formulation) | (Part B: Day 351) | ≥100 | 3000 | 300 | 0 | 300 | 600 |
| | Open-label | ≥30 to <40 | 2700 | 270 | 0 | 270 | 540 |
| | maintenance dose | ≥40 to <60 | 3000 | 300 | 0 | 300 | 600 |
| | (Part A: Days 197 | ≥60 to <100 | 3300 | 330 | 0 | 330 | 660 |
| | to 645, q8w) | ≥100 | 3600 | 360 | 0 | 360 | 720 |
| | (Part B: Days 365 | | | | | | |
| | to 813, q8w) | | | | | | |
| Open-Label | Open-label | ≥30 to <40 | 2700 | 27 | 0 | 27 | 54 |
| Extension | maintenance dose | ≥40 to <60 | 3000 | 30 | 0 | 30 | 60 |
| (100 mg/mL | (Part A: Days 197 | ≥60 to <100 | 3300 | 33 | 0 | 33 | 66 |
| formulation) | to 645, q8w) | ≥100 | 3600 | 36 | 0 | 36 | 72 |
| | (Part B: Days 365 | | | | | | |
| | to 813, q8w) | | | | | | |

^{a}Dose regimen is based on the participant's most recently recorded body weight. Contact the Medical Monitor if a participant's weight drops below 30 kg during the Randomized Controlled Period or Open-Label Extension Period.

a. Part A

For each participant in Part A, the entire treatment duration is up to 100 weeks, consisting of a Randomized Controlled Period (26 weeks), and an OLE Period (up to 74 weeks).

The Randomized Controlled Period is a double-blind, randomized, placebo-controlled period. Eligible participants are randomized 2:1 to receive blinded doses of ravulizumab or placebo starting on Day 1 and ending after pre-dose assessments on Week 26. Participants in the ravulizumab group receive a blinded loading dose of ravulizumab (10 mg/mL formulation) via IV infusion on Day 1, followed by a blinded maintenance dose at Week 2, Week 10, and Week 18. Participants in the placebo group receive a blinded matching placebo via IV infusion on Day 1, followed by a blinded matching placebo at Week 2, Week 10, and Week 18. Participants who discontinue study intervention are followed for key efficacy and safety assessments until the end of the Randomized Controlled Period, withdrawal of consent, or lost to follow-up, whichever is earlier.

The Randomized Controlled Period end after all pre-dose assessments have been completed at the Week 26 visit. The Open-Label Extension Period (OLE) Period begins when participants, that elect to continue treatment, receive the Week 26 study intervention infusion. Participants in both the placebo group and the ravulizumab group receive blinded doses of ravulizumab as follows: Participants in the placebo group switch to receive a blinded loading dose of ravulizumab (10 mg/mL formulation) at Week 26 (Table 3). Participants in the ravulizumab group receive a blinded ravulizumab dose (10 mg/mL formulation) of 900 mg at Week 26. The 900 mg dose at Week 26 is chosen to maintain complete C5 inhibition over the 2-week period until the next scheduled maintenance dose. This dosing maintains the treatment randomization blind of the Randomized Controlled Period. Starting at Week 28, all participants receive open-label ravulizumab maintenance doses (10 mg/mL or 100 mg/mL formulation) q8w until the end of the OLE Period (see Table 3).

b. Part B

For each participant in Part B, the entire treatment duration is up to 124 weeks, consisting of a Randomized Controlled Period (50 weeks), and an OLE Period (up to 74 weeks).

The Randomized Controlled Period is a double-blind, randomized, placebo-controlled period. Eligible participants are randomized 1:1 to receive blinded doses of ravulizumab or placebo starting on Day 1 and ending after pre-dose assessments on Week 50. Participants in the ravulizumab group receive a blinded loading dose of ravulizumab (10 mg/mL formulation) via IV infusion on Day 1, followed by a blinded maintenance dose at Week 2, then q8w through Week 42 (Table 3). Participants in the placebo group receive a blinded matching placebo via IV infusion on Day 1, followed by a blinded matching placebo at Week 2, then q8w through Week 42 (Table 3). Participants who discontinue study intervention are followed for key safety and efficacy assessments until the end of the Randomized Controlled Period, withdrawal of consent, or lost to follow-up, whichever is earlier.

The Randomized Controlled Period ends after all pre-dose assessments have been completed at the Week 50 visit. The OLE Period begins when participants who are eligible and elect to continue treatment receive the Week 50 study intervention infusion. Participants in both the placebo group and the ravulizumab group receives blinded doses of ravulizumab as follows. Participants in the placebo group switch to receive a blinded loading dose of ravulizumab (10 mg/mL formulation) at Week 50 (Table 3). Participants in the ravulizumab group receive a blinded ravulizumab dose (10 mg/mL formulation) of 900 mg at Week 50. The 900 mg dose at Week 50 is chosen to maintain complete C5 inhibition over the 2-week period until the next scheduled maintenance dose. This dosing maintains the treatment randomization blind of the Randomized Controlled Period. Starting at Week 52, all participants receive open-label ravulizumab maintenance doses (10 mg/mL or 100 mg/mL formulation) q8w (Table 3) until the end of the OLE Period.

c. Preparation/Handling/Storage/Accountability

Upon arrival of the study intervention at the study site, the study intervention kits are removed from the shipping container and stored in their original cartons under refrigerated conditions at 2° C. to 8° C. (35° F. to 47° F.) and protected from light. Study interventions are not frozen. Study interventions must be stored in a secure, limited-access storage area with temperature monitored daily. Infusions of study intervention are prepared using aseptic technique. Ravulizumab and placebo are further diluted in a 1:1 ratio with compatible diluent. Ravulizumab and placebo are filtered with a 0.2 micron filter during infusion.

### 6. Concomitant Therapy

Any medication (including over the counter or prescription medicines, vitamins, and/or herbal supplements), vaccine, or other specific categories of interest that the participant is receiving at the time of enrollment or receives during the study must be recorded along with: (1) reason for use, (2) dates of administration, including start and end dates, and (3) dosage information including dose and frequency.

Participants who continue allowed DM treatments as concomitant therapy are required to have received those treatments for at least the amount of time described in Table 4 before the Day 1 Visit and are required to remain on a stable dose during the Randomized Controlled Period. Maximally allowed stable doses are described in Table 4. In some cases, stable doses of allowed DM treatments may be decreased due to safety and/or tolerability issues.

During the OLE Period, doses of allowed DM treatments may be changed or discontinued. New allowed DM treatments may be started in the OLE period. Medications listed Table 5 are prohibited during the OLE period. Any change in medication is properly documented in the CRF.

TABLE 4
Allowed Concomitant DM Treatments

| | | | | | Dose |
|---|---|---|---|---|---|
| | | Treatment | Maximally | | Modifications |
| | Duration of | Duration | Allowed Stable | | Permitted During |
| | Stable Dose | Required | Dose as | | Randomized |
| | Before Day 1 | Before Day 1 | Concomitant | | Controlled |
| Medication | Visit | Visit | therapy | | Period^{a} |
| Azathioprine | 4 weeks | 12 weeks | 2.5 | mg/kg | None |
| Cyclosporine | 4 weeks | 12 weeks | 2 | mg/kg | None |
| Glucocorticoid | 4 weeks | 4 weeks | 20 mg daily | | None |
| | | | Prednisone | | |
| | | | equivalent^{b} | | |
| Intramuscular | Not | Not | 80 | mg/mL | Only 2 injections |
| glucocorticoids | Applicable | Applicable | | | allowed |
| Hydroxychloroquine | 4 weeks | 12 weeks | 400 | mg daily | None |
| Leflunomide | 4 weeks | 12 weeks | 20 | mg daily | None |
| Methotrexate | 4 weeks | 12 weeks | 25 | mg/week | None |
| Mycophenolate | 4 weeks | 12 weeks | 3000 | mg daily | None |
| mofetil/mycophenolic | | | | | |
| acid | | | | | |
| Sulfasalazine | 4 weeks | 12 weeks | 3 | g daily | None |
| Tacrolimus | 4 weeks | 12 weeks | 0.2 | mg/kg | None |

^{a}Unless medically necessary or due to participant safety reasons.
^{b}Prednisone equivalent medications and doses are described in the Study Manual.

Alternatively, participants can choose to discontinue previous DM treatments before the Day 1 Visit, but are required to complete an appropriate washout period, as described in Table 5.

TABLE 5
Washout Periods Required Before Day 1 Visit

| Medication | Washout | |
|---|---|---|
| Methotrexate | 4 | weeks |
| Azathioprine | 4 | weeks |
| Cyclosporine | 4 | weeks |
| Tacrolimus | 4 | weeks |
| Leflunomide | 3 | months |
| Mycophenolate mofetil/mycophenolic acid | 4 | weeks |
| Glucocorticoid | 4 | weeks |
| Hydroxychloroquine | 8 | weeks |
| Sulfasalazine | 4 | weeks |
| Intramuscular glucocorticoids | 4 | weeks |

The following medications can be taken as needed, but should not be administered within
72 hours prior to the study visit: Antihistamines (oral and topical), Ibuprofen,
Acetaminophen,
Anti-pruritics, and Topical steroids (1% hydrocortisone).
   Vitamin B12, vitamin E, creatine, coenzyme Q10, and biotin supplements are permitted
in this study. Participants who take any or all of these supplements should be on a stable dose beginning 14 days prior to the first dose of study intervention and remain on a stable dose for the duration of the Randomized Controlled Period of the study unless alteration in dose is deemed medically necessary. All other vitamins and supplements are permitted in this study. Participants are encouraged to remain on stable dosing for the duration of the Randomized Controlled Period of the study.

The following medications and therapies are prohibited during the study: (1) treatments listed in Table 6 (participants must complete the appropriate washout period for these treatments before the Day 1 Visit), (2) Eculizumab or other complement inhibitory agent, and (3) any off-label usage of an approved product currently under investigational use for the treatment of DM during the study.

TABLE 6
Washout Periods Required Before
Day 1 for Prohibited Medications

| Medication | Washout | |
|---|---|---|
| intravenous immunoglobulin (IVIg)/ | 3 | months |
| subcutaneous immunoglobulin (SCIg) | | |
| IV glucocorticoids | 4 | weeks |
| Corticotropin injection | 8 | weeks |
| Cyclophosphamide | 3 | months |
| Rituximab | 6 | months |
| Infliximab | 8 | weeks |
| Adalimumab | 3 | months |
| Etanercept | 4 | weeks |
| Tofacitinib or other Janus Kinase (JAK) inhibitors | 4 | weeks |
| Ruxolitinib | 4 | weeks |
| Anakinra | 1 | week |

Participants who require prohibited medications at any point during the study are discontinued from the study intervention.

To allow for sufficient time for the study drug to become effective, it is recommended that participants not receive any rescue therapy during the first 10 weeks of receiving the study intervention during the Randomized Controlled Period. At any time during the Randomized Controlled Period, if the participant requires rescue therapy with increased dose or initiated new DM treatments (glucocorticoid and/or ISTs), the participant is discontinued from study intervention.

Participants in the Randomized Controlled Period who discontinue study intervention early for any reason and agree to remain in the study are followed up for safety and efficacy assessments for the duration of the Randomized Controlled Period. If the decision to discontinue study intervention falls outside of a study visit, participants are expected to return to an unscheduled visit as soon as possible within a week.

Participants in the Randomized Controlled Period who discontinue study intervention early and do not agree to continue in the study are expected to complete the study visit (if at the site for a scheduled visit) or return for an unscheduled visit as soon as possible within a week and an ET Visit 8 weeks after the participant's last dose of study intervention. In addition, a follow-up phone call 20 weeks after the participant's last dose of study intervention is performed to collect concomitant medications, non-pharmacologic therapies and procedures, and AEs. Patients who discontinue study intervention during the Randomized Controlled Period are not eligible for the OLE Period. Glucocorticoid tapers are allowed as needed throughout the OLE Period.

### 7. Study Assessments and Procedures

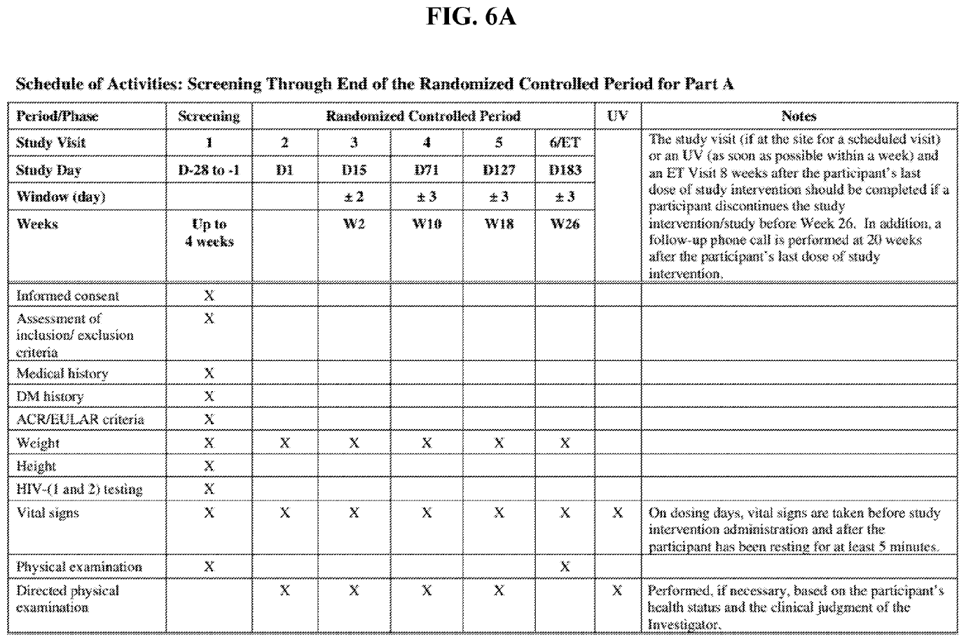

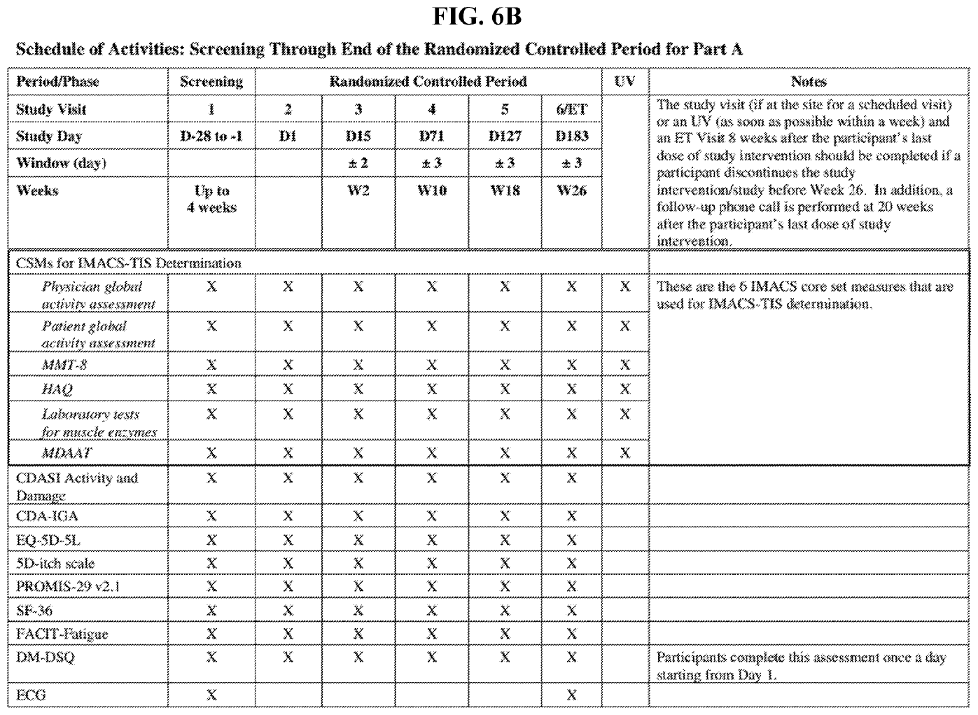

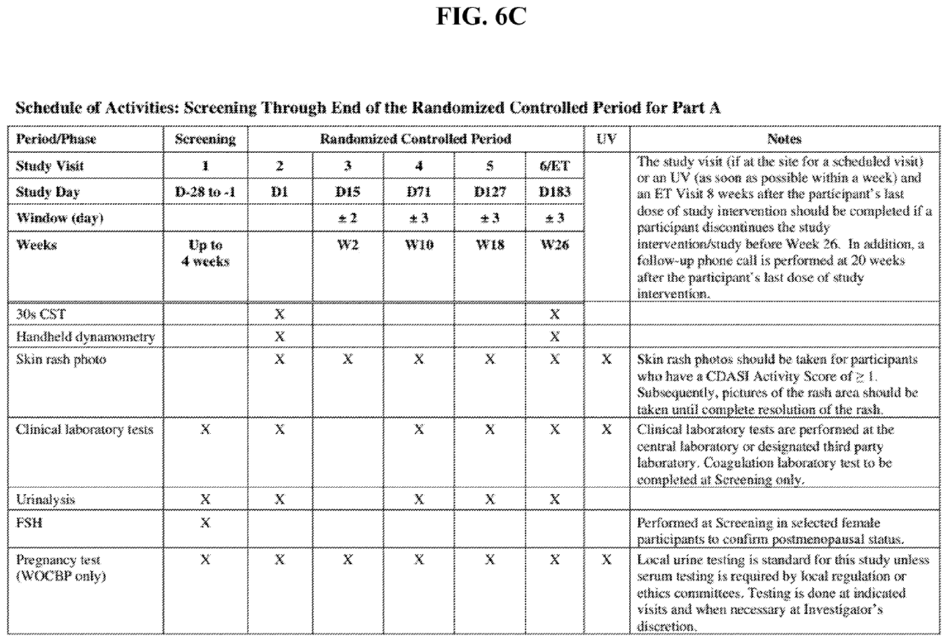

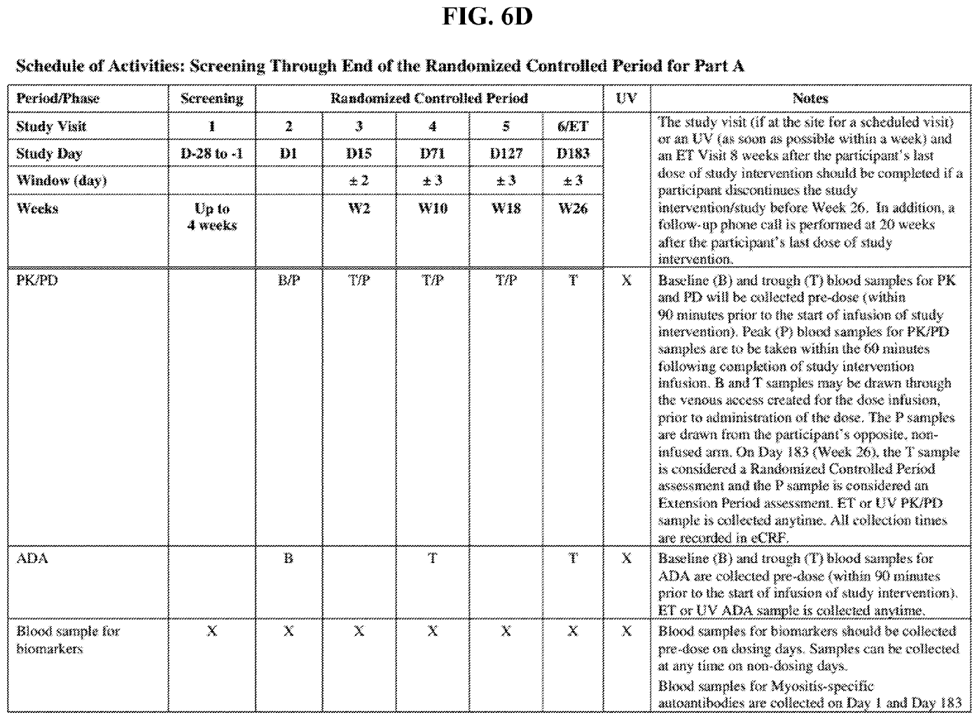

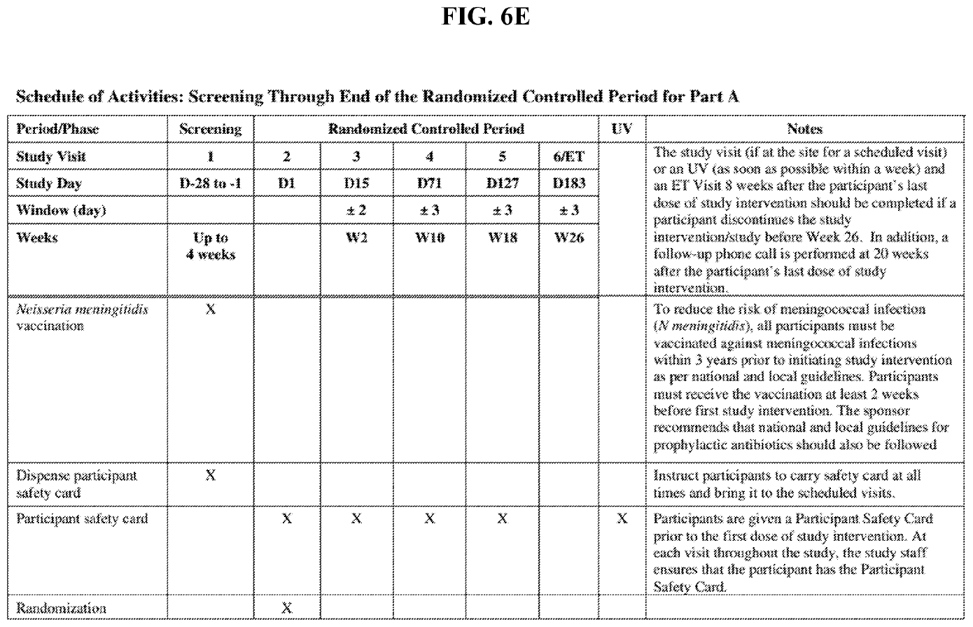

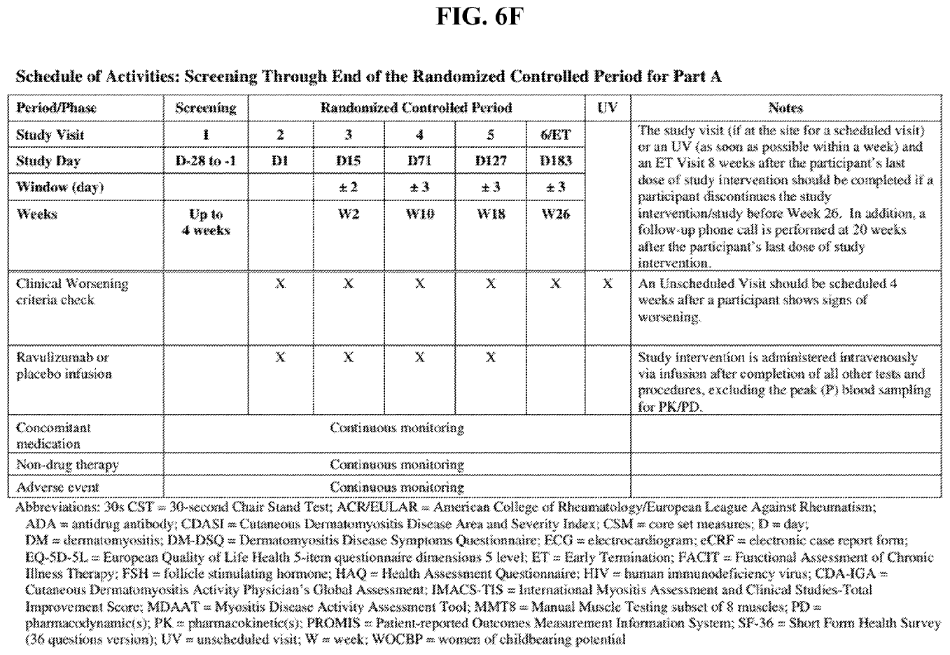

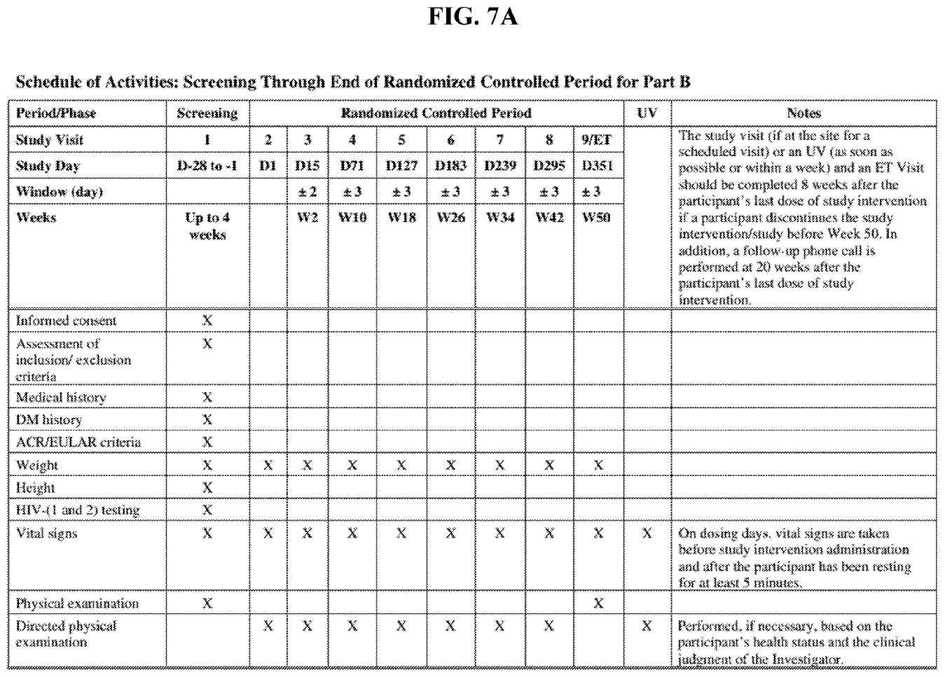

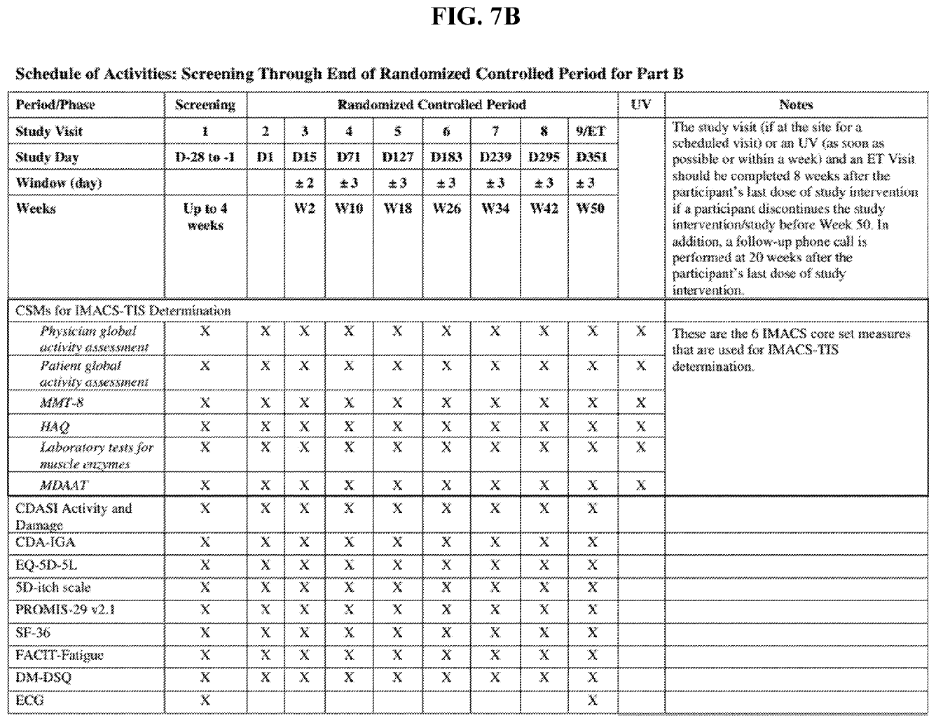

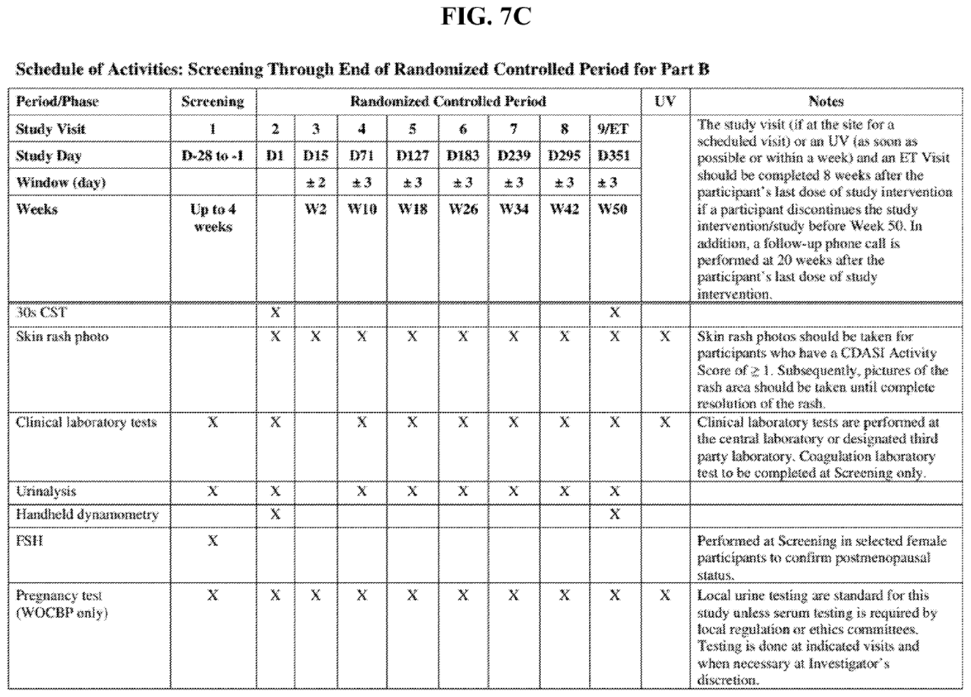

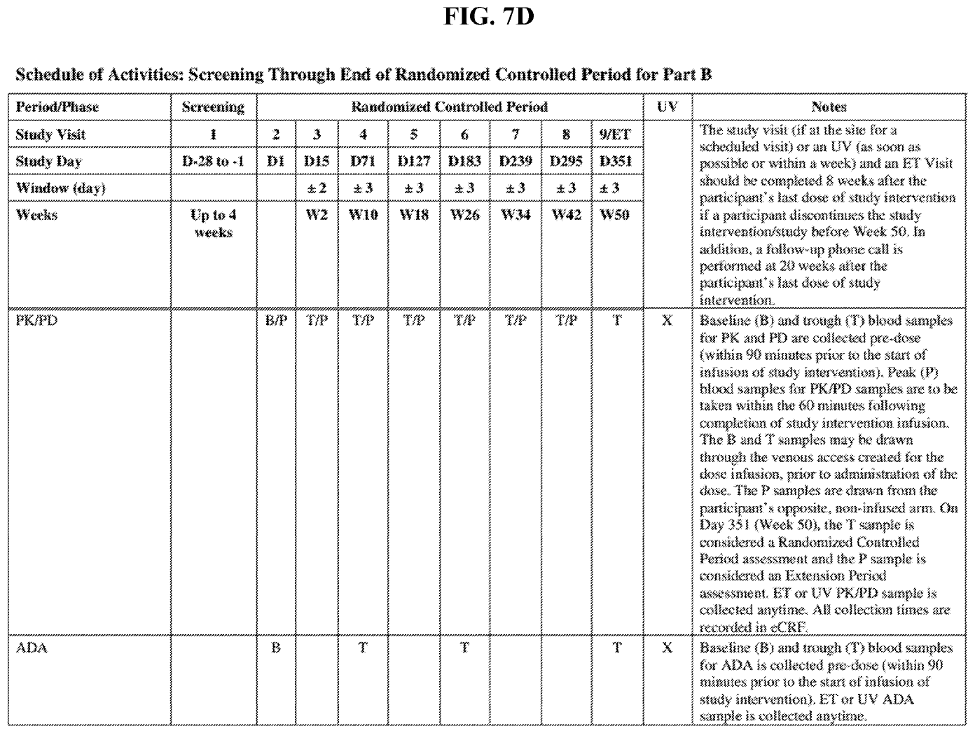

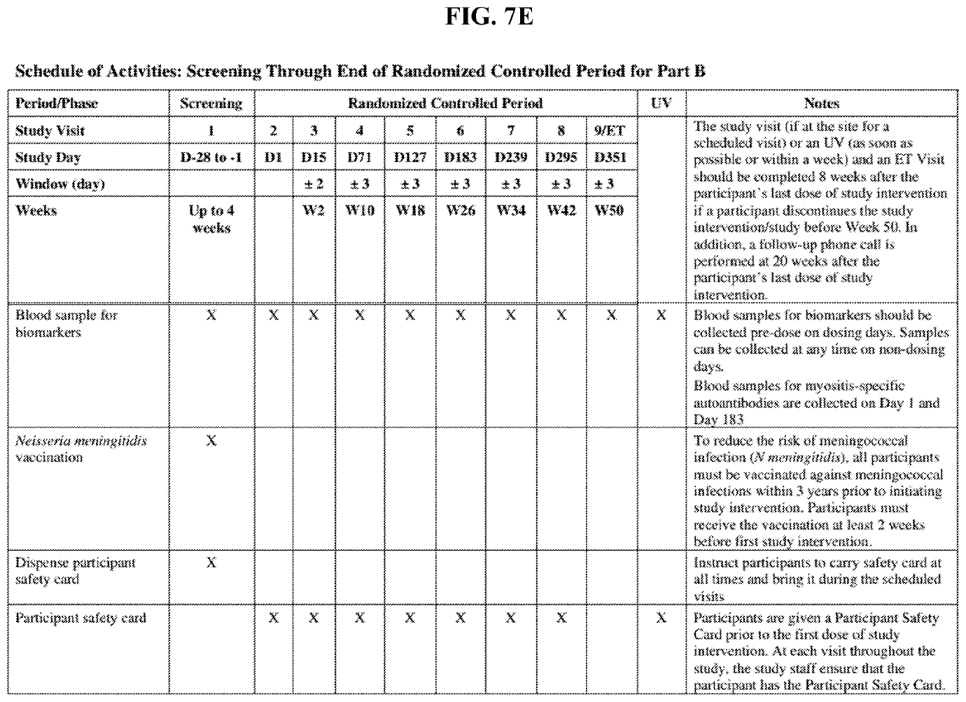

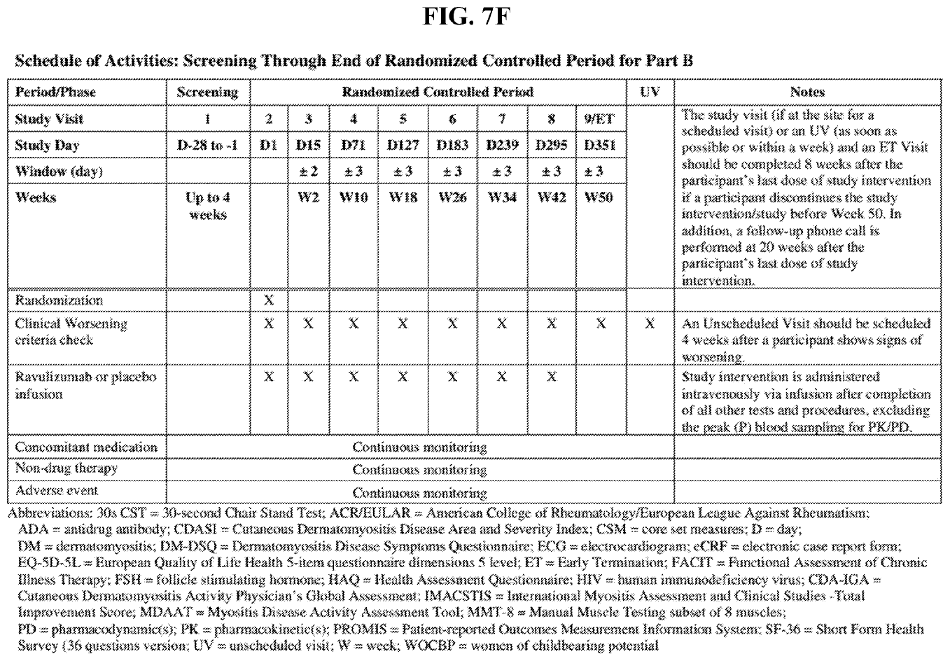

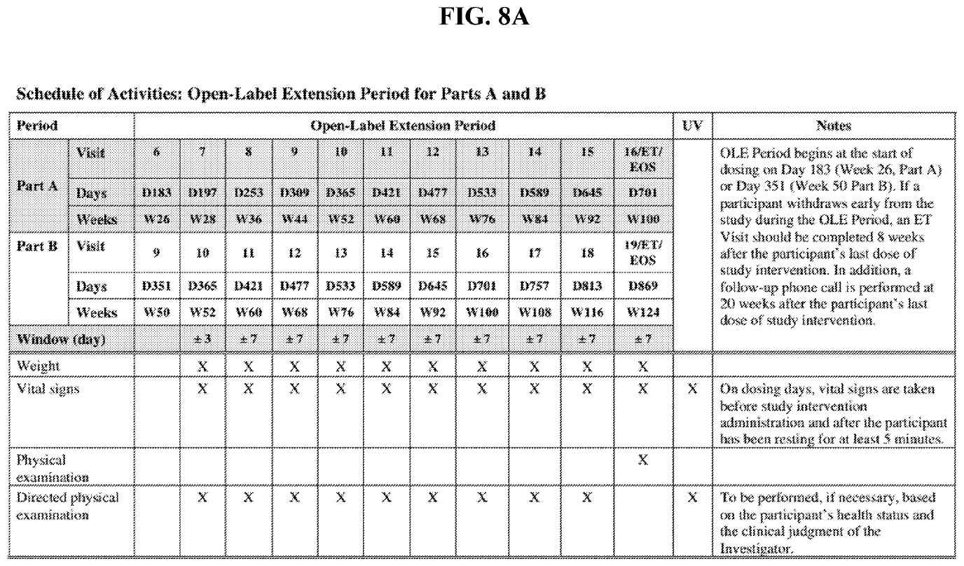

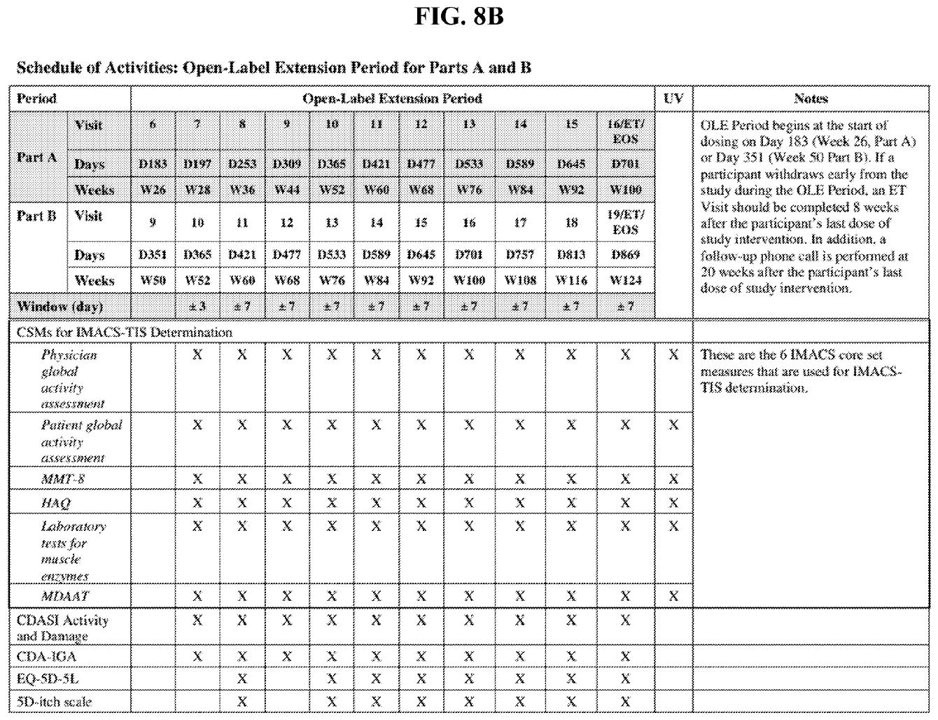

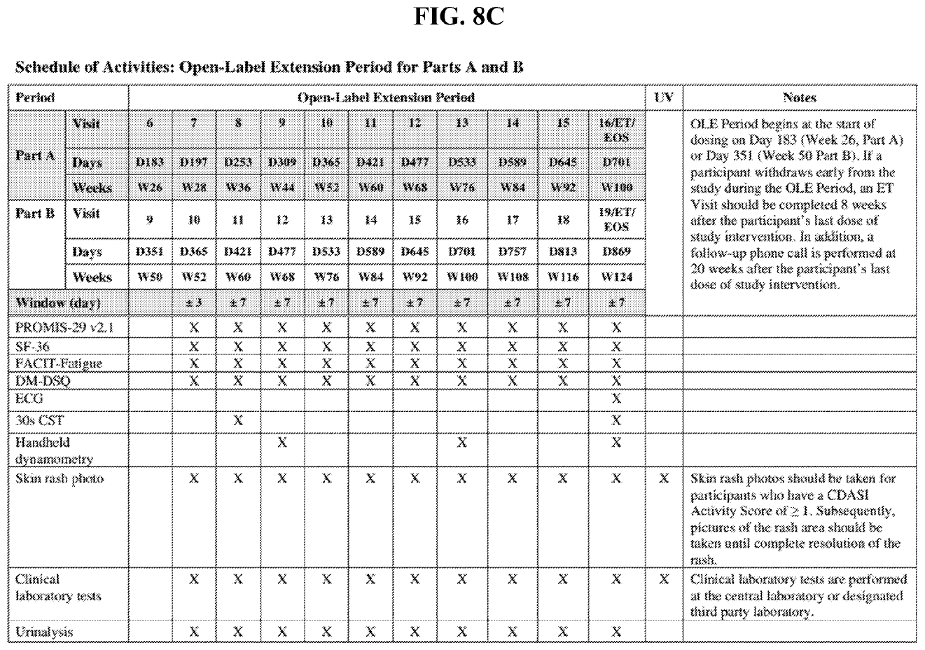

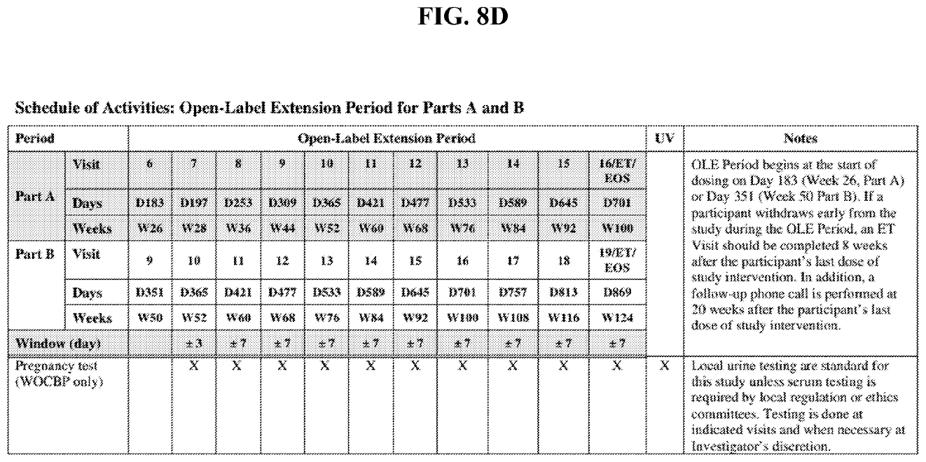

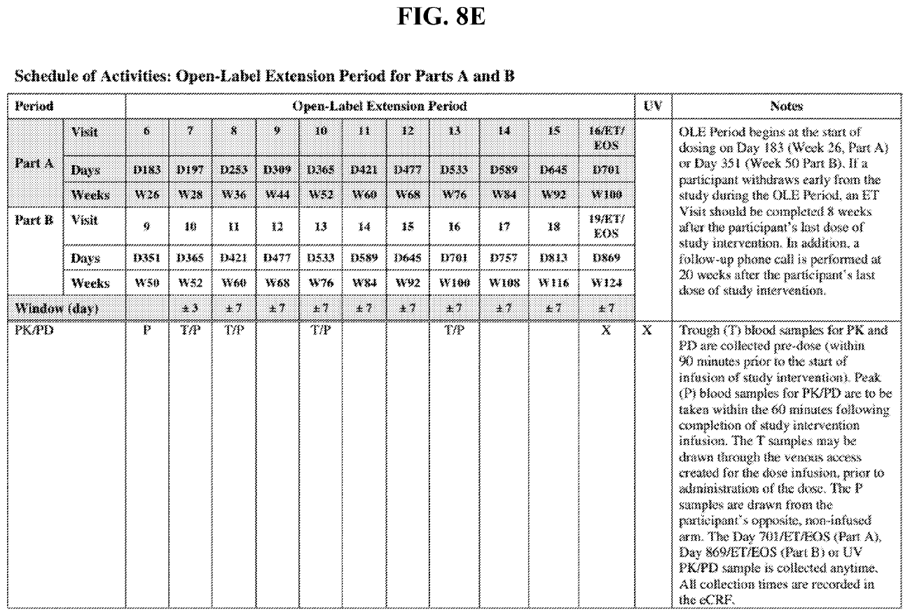

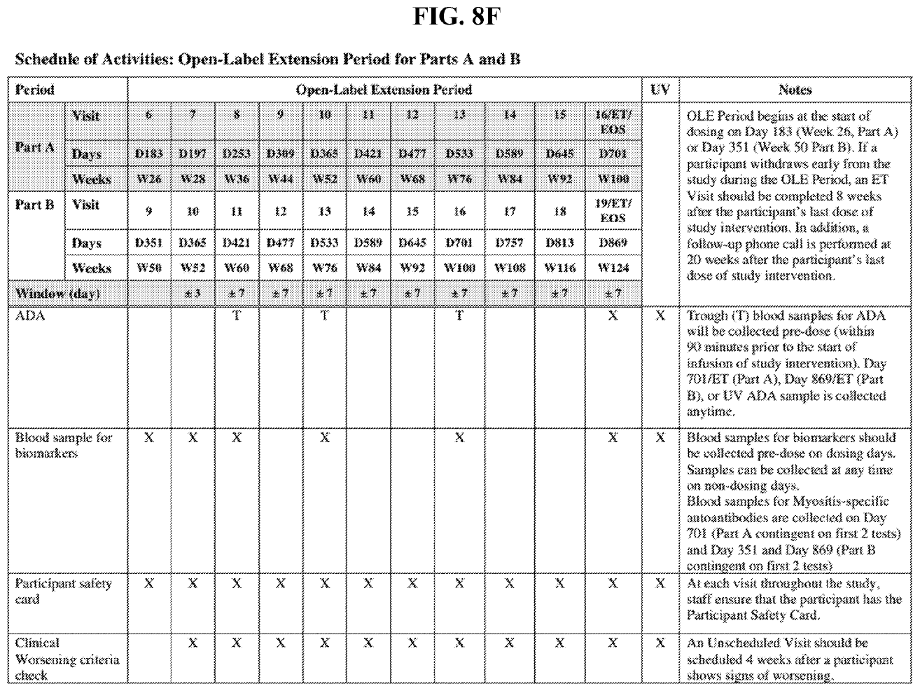

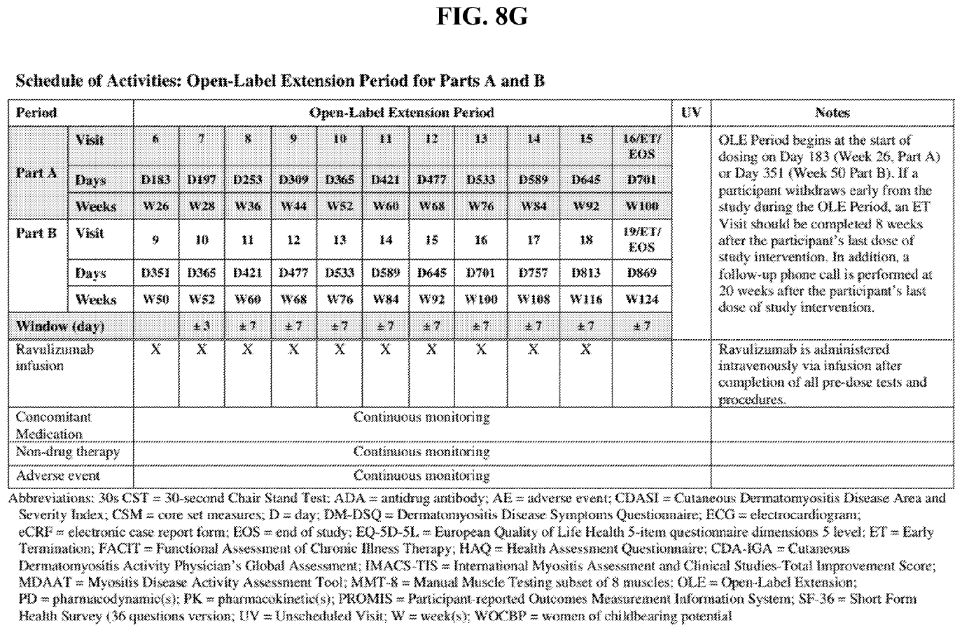

Study procedures and their timing are summarized in the Schedule of Activities. The Schedule of Activities for Part A (screening through end of the randomized controlled period) is set forth in FIGS. 6A-6F. The Schedule of Activities for Part B (screening through end of the randomized controlled period) is set forth in FIGS. 7A-7F. The Schedule of Activities for the Open-Label Extension Period for Parts A and B is set forth in FIGS. 8A-8G

Protocol waivers or exemptions are not allowed. All screening evaluations must be completed and reviewed to confirm that potential participants meet all eligibility criteria. The Investigator maintains a screening log to record details of all participants screened and to confirm eligibility or record reasons for screening failure, as applicable. Procedures conducted as part of the participant's routine clinical management (e.g., blood count) and obtained before signing of the Informed Consent Form (ICF) may be utilized for screening or baseline purposes provided the procedures met the protocol-specified criteria and were performed within the time frame defined in the Schedule of Activities.

a. Efficacy Assessments

All efficacy assessments, not completed by the participants themselves, should preferably be performed by the same study staff member throughout the study. The following are exemplary efficacy assessments.

i. International Myositis Assessment and Clinical Studies (IMACS) Core Set Measures (CSM)

The IMACS group developed a consensus on outcome measures and definitions of improvement that should be used in clinical trials for DM (see Aggarwal R, et al., Arthritis Rheumatol. 2017; 69(5):898-910). The CSM are described in the following subsections.

ii. Manual Muscle Testing 8 Muscles Group (MMT-8)

The purpose of the MMT-8 is to measure muscle strength as part of the physical examination. It includes a subset of 8 muscle groups: neck flexors, deltoids, biceps, wrist, extensors, gluteus maximus and medius, quadriceps, and ankle dorsiflexors (see Rider L G, et al., Measures of adult and juvenile dermatomyositis, polymyositis, and inclusion body myositis: Physician and Patient/Parent Global Activity, Manual Muscle Testing (MMT), Health Assessment Questionnaire (HAQ)/Childhood Health Assessment Questionnaire (C-HAQ), Childhood Myositis Assessment Scale (CMAS), Myositis Disease Activity Assessment Tool (MDAAT), Disease Activity Score (DAS), Short Form 36 (SF-36), Child Health Questionnaire (CHQ), physician global damage, Myositis Damage Index (MDI), Quantitative Muscle Testing (QMT), Myositis Functional Index-2 (FI-2), Myositis Activities Profile (MAP), Inclusion Body Myositis Functional Rating Scale (IBMFRS), Cutaneous Dermatomyositis Disease Area and Severity Index (CDASI), Cutaneous Assessment Tool (CAT), Dermatomyositis Skin Severity Index (DSSI), Skindex, and Dermatology Life Quality Index (DLQI). Arthritis Care Res (Hoboken). 2011b; 63 Suppl 11:S118-157). MMT-8 is administered at timepoints listed in the Schedule of Assessments.

iii. Physician Global Activity Assessment

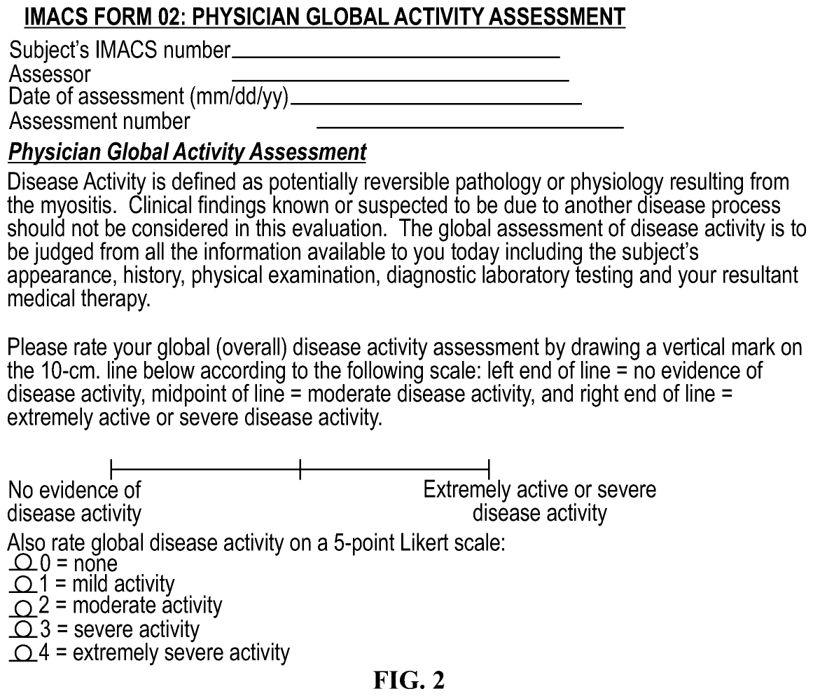

The physician global activity assessment provides an overall rating of disease activity related to myositis. Disease activity is judged by the physician based on all information available at the time of evaluation, including the participant's appearance, medical history, physical examination, laboratory testing, and prescribed medical therapy. The global disease activity score is recorded on a 10-cm VAS anchored at the end points and the middle. The physician global activity assessment is completed at timepoints listed in Schedule of Assessments. An exemplary physician global activity assessment is set forth in FIG. 2.

iv. Patient Global Activity Assessment

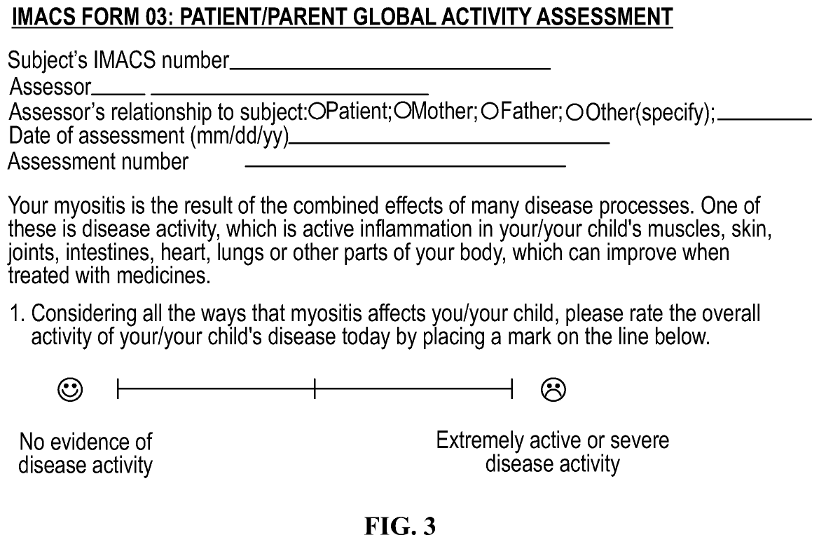

The patient global activity assessment provides an overall rating of disease activity related to myositis from the participant's perspective. Participants are asked to consider all of the active inflammation in their own muscles, skin, joints, intestines, heart, lungs, or other parts of the body that can improve with treatment. The patient global disease activity score is recorded on a 10-cm VAS that contains a smiley face at the 0-cm anchor and a sad face at the 10 cm anchor to help participants understand the scale (see Rider L G, et al., Arthritis Care Res (Hoboken). 2011b; 63 Suppl 11:S118-157). The patient global activity assessment is completed at timepoints listed in the Schedule of Activities. An exemplary patient global activity assessment is set forth in FIG. 3.

v. Health Assessment Questionnaire (HAQ)

The HAQ is a brief self-report questionnaire that assesses physical function pertaining to activities of daily living in a variety of domains (see Rider L G, et al., Arthritis Care Res (Hoboken). 2011b; 63 Suppl 11:S118-157). The Full Five Dimension HAQ includes the following domains: disability, discomfort and pain, drug side effects, and dollar costs. In this study, the HAQ Disability Index is completed at timepoints listed in the Schedule of Activities.

vi. Muscle Enzymes

As part of the IMACS CSM, laboratory tests to measure serum activities of muscle associated enzymes including CK, ALT and AST, LDH, and aldolase are performed. The most abnormal serum muscle enzyme value at baseline are used (Aggarwal R, et al., 2016 American College of Rheumatology/European League Against Rheumatism Criteria for Minimal, Moderate, and Major Clinical Response in Adult Dermatomyositis and Polymyositis: An International Myositis Assessment and Clinical Studies Group/Paediatric Rheumatology International Trials Organisation Collaborative Initiative. Arthritis Rheumatol. 2017; 69(5):898-910). Muscle enzymes are assessed at the timepoints listed in the Schedule of Activities.

vii. Myositis Disease Activity Assessment Tool (MDAAT)

The MDAAT assesses disease activity of extramuscular organ systems and muscle in patients with DM. It is a combined tool that includes the Myositis Disease Activity Assessment VAS (MYOACT) and the Myositis Intention to Treat Activities Index (MITAX). The MITAX assesses specific manifestations in 7 organs/systems: constitutional, cutaneous, skeletal, gastrointestinal, pulmonary, cardiac, and muscle. The MYOACT consist of a 10-cm VAS for each organ system and overall. The MDAAT is administered in-person and completed by the clinician (see Rider L G, et al., Arthritis Care Res (Hoboken). 2011b; 63 Suppl 11:S118-157). MDAAT is completed at the timepoints listed in the Schedule of Assessments.

viii. Cutaneous Dermatomyositis Disease Area and Severity Index (CDASI)

The CDASI is an instrument that separately measures activity and damage in the skin of DM patients. It is a 1-page instrument that contains 3 activity measures (erythema, scale, and erosion/ulceration) and 2 damage measures (poikiloderma and calcinosis). CDASI is completed by the Clinician or Clinician-Investigator while examining the participant (see Rider L G, et al., Arthritis Care Res (Hoboken). 2011b; 63 Suppl 11:S118-157). CDASI is completed at the timepoints listed in the Schedule of Activities.

ix. Cutaneous Dermatomyositis Activity Physician's Global Assessment (CD-IGA)

The CD-IGA is a scale that was created to measure disease severity in patients with skin disease. It is a 5-point scale (0=clear; 1=almost clear; 2=mild; 3=moderate; 4=severe) with morphologic descriptors for each score. The CD-IGA is completed by the Investigator and is used to describe the overall appearance of lesions at a given time point. The CD-IGA is completed at the timepoints listed in the Schedule of Assessments.

x. EuroQoL 5 Dimensions (EQ-5D-5L)

The European Quality of Life Health 5-item questionnaire dimensions 5 level (EQ 5D 5L) is a self-assessed, standardized instrument to measure health related quality of life and has been used in a wide range of health conditions (Schrag A, et al., Journal of neurology, neurosurgery, and psychiatry. 2000; 69(1):67-73). The EQ 5D 5L consists of 2 pages: the EQ-5D-5L descriptive system and the EQ VAS. The EQ-5D-5L is completed at time points specified in the Schedule of Assessments or if a participant is not able to attend the scheduled onsite visit, EQ-5D-5L can be assessed via a phone call.

xi. EQ-5D-5L Descriptive System

The descriptive system is a 5-component scale including mobility, self-care, usual activities, pain/discomfort, and anxiety/depression. Each level is rated on a scale that describes the degree of problems in that area.

xii. EQ Visual Analog Scale

The EQVAS is an overall health state scale where the participant selects a number between 0 and 100 to describe the condition of their health, with 100 being ‘The best health state you can imagine’ and 0 being ‘The worst health state you can imagine’.

This information can be used as a quantitative measure of health outcome as judged by the individual respondents. Previously published studies by EuroQol Group members showed preliminary evidence of the instrument's feasibility, reliability, and validity.

xiii. 5D-Itch Scale

The 5D-itch scale is a multidimensional questionnaire, which includes the following 5 dimensions: degree, duration, direction, disability, and distribution (see, e.g., Elman et al., Br. J. Dermatol. 2010 March; 162(3):587-93). The 5D itch scale is completed by the participant at the timepoints described in the Schedule of Activities.

xiv. Patient-Reported Outcomes Measurement Information System (PROMIS) 29 v2.1 Tool

The PROMIS 29 v2.1 profile assesses pain intensity using a single 0-10 numeric rating item and 7 health domains (physical function, fatigue, pain interference, depressive symptoms, anxiety, ability to participate in social roles and activities, and sleep disturbance) using 4 items per domain (see, e.g., Hays et al., Qual. Life Res. 2018 July; 27(7):1885-1891). PROMIS-29 v2.1 is completed by the participant at the timepoints described in the Schedule of Activities.

xv. Short Form Health Survey (36 Questions Version) (SF-36)

The SF-36 (Version 2.0) is a 36-item self-report of health-related quality of life (see Stewart A L, et al., Med Care. 1988; 26(7):724-735; and Ware et al., Med Care. 1992; 30(6):473-483). It contains 8 subscales measuring different domains of health-related quality of life: physical functioning, role limitations due to physical problems, bodily pain, general health perceptions, vitality, social functioning, role limitations due to emotional problems, and mental health. The SF-36 is conducted at screening and at timepoints specified per the Schedule of Activities. The 2 summary scores are the physical component summary and the mental component summary. There is no single overall score for the SF-36.

xvi. Functional Assessment of Chronic Illness Therapy-Fatigue (FACIT-Fatigue)

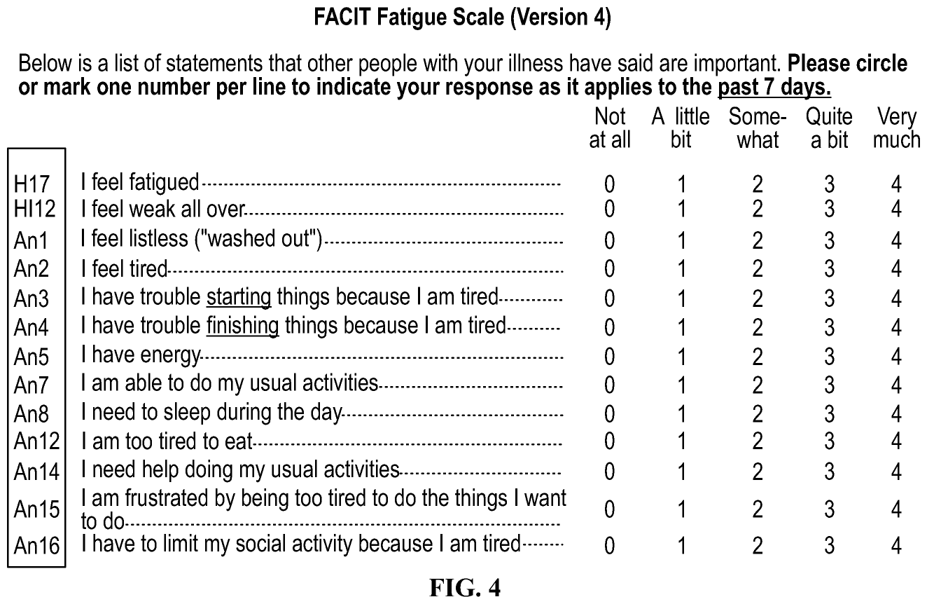

The FACIT-Fatigue Scale (Version 4) is a short, 13-item, self-reported, easy to administer tool that measures an individual's level of fatigue during their usual daily activities over the past week (see, e.g., Tennant, K., Supportive Care in Cancer 23.5 (2015): 1355-1364). The level of fatigue is measured on a five-point Likert-type scale (0=not at all fatigued; 1=a little bit fatigued, 2=somewhat fatigued, 3=quite a bit fatigued, and 4=very much fatigued). All items contribute to the sum score with equal weight. FACIT Fatigue are completed at timepoints specified in the Schedule of Activities. An exemplary FACIT Fatigue is set forth in FIG. 4.

xvii. Dermatomyositis Disease Symptoms Questionnaire (DM-DSQ)

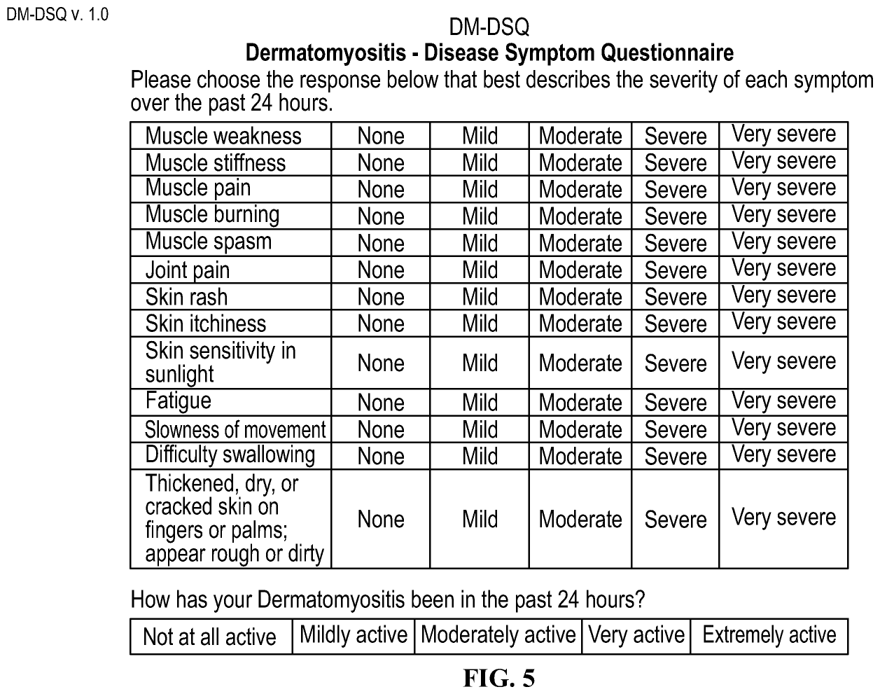

The Dermatomyositis Disease Symptom Questionnaire (DM-DSQ) version 1.0 is a 14-item PRO instrument designed to assess symptoms relevant to the experience of patients with DM in the past 24 hours. Each item was generated based on a corresponding concept extracted from published qualitative literature (interviews or focus groups with patients) or from direct patient input (minutes from meeting with patients describing their disease and video testimonials of patients from the Myositis Association). There are 13 symptom questions that are rated according to their severity on a 5-point response scale (None, Mild, Moderate, Severe, and Very severe) and one overall question about disease activity rated on a 5-point response scale (Not at all active, Mildly active, Moderately active, Very active, Extremely active). DM-DSQ is completed at the timepoints specified in the Schedule of Activities. An exemplary DM-DSQ is set forth in FIG. 5.

xviii. 30-Second Chair Stand Test (30s CST)

The 30s CST is a procedure that assesses proximal muscle function (see Agarwal S, Kiely P D, Rheumatology (Oxford). 2006; 45(7):874-879; and Rider L G, et al., Nat. Rev. Rheumatol. 2018; 14(5):303-318). During the test a participant is asked to rise from a standard-height chair and to sit down as many times as possible in 30 seconds. The 30s CST is assessed at the timepoints specified in the Schedule of Activities.

xix. Handheld Dynamometry

Handheld dynamometry (see Rider L G, et al., Nat. Rev. Rheumatol. 2018; 14(5):303-318) is a procedure for quantitative strength testing. This testing is conducted by the Investigator or any designee who has been properly trained for the quantitative muscle strength evaluation. When possible, it is highly recommended that all assessments be performed by the same assessor. Muscle strength testing is performed on prespecified muscles in the upper and lower extremities bilaterally and the force measurements recorded. Handheld dynamometry is assessed at screening and timepoints specified in the Schedule of Activities.

xx. Skin Rash Photography

High-resolution digital images of skin rashes are taken at the timepoints specified in the Schedule of Activities.

xxi. Home Electronic Patient-Reported Outcomes

For Part A, participants are asked to complete the following PROs at the frequencies described in the Schedule of Activities: DM-DSQ, HAQ, PROMIS-29 v2.1, FACIT-Fatigue, and SF-36.

These PROs are completed using an electronic device (e.g., iPad). Further details of the selected PROs and the process associated with completing the PROs are included in a separate electronic PRO (ePRO) Manual.

b. Safety Assessments

Planned time points for all safety assessments are provided in the Schedule of Activities.

As with any complement C5 inhibition, the use of ravulizumab increases the participant's susceptibility to meningococcal infection (N meningitidis). To reduce the risk of meningococcal infection, all participants must be vaccinated against meningococcal infection within the 3 years before or at the time of initiating study intervention. Participants must receive vaccination at least 2 weeks before first study intervention. Participants must be vaccinated or revaccinated according to current national vaccination guidelines or local practice for vaccination use with complement inhibitors (e.g., eculizumab, ravulizumab). Vaccines against serotypes A, C, Y, W135, and B, where available, are recommended to prevent common pathogenic meningococcal serotypes. Vaccination may not be sufficient to prevent meningococcal infection. Consideration should be given according to official guidance and local practice on the appropriate use of antibacterial agents. All participants should be monitored for early signs of meningococcal infection, evaluated immediately if infection is suspected, and treated with appropriate antibiotics, if necessary.

A complete physical examination includes, at a minimum, assessments of the following organs/body systems: skin, head, ears, eyes, nose, throat, neck, lymph nodes, chest, heart, abdomen, extremities, and musculoskeletal. A directed physical examination includes, at minimum, a body-system relevant examination based upon Investigator judgment and participant symptoms. Examiners should pay special attention to clinical signs related to previous serious illnesses. For consistency, all efforts should be made to have the physical examination performed by the same qualified study staff at each study visit. Additional physical examinations can be performed as medically indicated during the study at the Investigator's discretion.

Body weight is measured in pounds or kilograms. Height is measured in inches or centimeters.

Temperature (° C. or ° F.), pulse rate, respiratory rate, and systolic and diastolic blood pressure (mm Hg), and pulse oximetry are assessed.

Blood pressure and pulse measurements are assessed seated with a completely automated device. Manual techniques are used only if an automated device is not available.

Blood pressure and pulse measurements should be preceded by at least 5 minutes of rest for the participant in a quiet setting without distractions (e.g., television, cell phones). Ideally, the same arm for each participant should be used for measurements. 02 saturation (%) is collected using pulse oximetry.

Single 12-lead electrocardiogram (ECG) is performed at protocol specified visits in the Schedule of Activities using an ECG machine to obtain heart rate and measures of PR, QRS, QT, and QT intervals. Participants must be supine for approximately 5 to 10 minutes before ECG collection and remain supine but awake during ECG collection.

The clinical laboratory tests set forth in Table 7 are performed by a central laboratory. Additional tests may be performed at any time during the study as determined necessary by the Investigator or required by local regulations

TABLE 7
Laboratory Assessments

| Laboratory | | | |
|---|---|---|---|
| Assessments | Parameters | | |
| Hematology | Platelet count | Red Blood Cell (RBC) | White Blood Cell |
| | RBC count | indices: | (WBC) count with |
| | Hemoglobin | Distribution width | differential: |
| | Hematocrit | Mean corpuscular | Neutrophils |
| | | volume | Lymphocytes |
| | | Mean corpuscular | Monocytes |
| | | Hemoglobin | Eosinophils |
| | | % Reticulocytes | Basophils |
| Clinical | blood urea nitrogen | Creatine kinase | Total and direct |
| Chemistry | (BUN) | Aldolase | bilirubin |
| | C-reactive protein | LDH | Total protein |
| | Creatinine | aspartate | Albumin |
| | Chloride | aminotransferase | Uric acid |
| | Potassium | (AST) | |
| | Bicarbonate | alanine | |
| | Sodium | aminotransferase | |
| | Glucose (non-fasting) | (ALT) | |
| | Calcium | Alkaline phosphatase | |
| | Magnesium | Gamma | |
| | | glutamyltransferase | |

| | |
|---|---|
| Coagulation | International normalized ratio, partial thromboplastin time, prothrombin time |
| Urinalysis | Appearance, color, specific gravity, pH, glucose, protein, leukocyte esterase, |
| | blood, ketones, bilirubin, urobilinogen, nitrite, microscopic examination (if |
| | blood or protein is abnormal) |
| Other | Serum/urine β-human chorionic gonadotropin (HCG) pregnancy test (as |
| screening | needed for participants of childbearing potential)^{a} |
| tests | Serum follicle-stimulating hormone test to be performed at Screening |
| | in selected female participants to confirm postmenopausal status |
| | human immunodeficiency virus (HIV)-1 and HIV-2 antibodies |
| Complement | Free C5 |
| activity | Total C5 |
| Other | Antidrug antibodies |

^{a}Local urine testing is standard for the protocol unless serum testing is required by local regulation or ethics committees.

The Investigator must review the laboratory report, document this review, and record any clinically relevant changes occurring during the study in the Adverse Event section of the eCRF. The laboratory reports must be filed with the source documents. Clinically significant abnormal laboratory findings are those which are not associated with the underlying disease, unless judged by the Investigator to be more severe than expected for the participant's condition.

All laboratory tests with values considered clinically significantly abnormal during participation in the study or within 8 weeks after the final dose of study intervention should be repeated until the values return to normal or baseline or are no longer considered clinically significant.

Pregnancy testing must be performed on all women of childbearing potential.

### 8. Adverse Events (AEs) and Serious Adverse Events (SAEs)

Adverse events are reported to the Investigator by the participant (or, when appropriate, by a caregiver, surrogate, or the participant's legally authorized representative). An AE is defined as any untoward medical occurrence in a participant or clinical investigation participant administered a pharmaceutical product and which does not necessarily have to have a causal relationship with this treatment (ICH E2A). An AE can, therefore, be any unfavorable and unintended sign (including an abnormal laboratory finding), symptom, or disease [new or exacerbated] temporally associated with the use of the study intervention, whether or not considered related to the study intervention.

The following are events that meet the definition of an adverse event:
1. Any abnormal laboratory test results (hematology, clinical chemistry, or urinalysis) or other safety assessments (e.g., ECG, radiological scans, vital signs measurements), including those that worsen from baseline, considered clinically significant in the medical and scientific judgment of the Investigator (i.e., not related to progression of underlying disease).
2. Exacerbation of a chronic or intermittent pre-existing condition, including either an increase in frequency and/or intensity of the condition.
3. New conditions detected or diagnosed after study intervention administration even though it may have been present before the start of the study.
4. Signs, symptoms, or the clinical sequelae of a suspected drug-drug interaction.
5. Signs, symptoms, or the clinical sequelae of a suspected overdose of either study intervention or a concomitant medication. Overdose per se is not reported as an AE/SAE unless it is an intentional overdose taken with possible suicidal/self-harming intent. Such overdoses should be reported regardless of sequelae.

The following events do not meet the definition of an adverse event:
1. Medical or surgical procedure (e.g., endoscopy, appendectomy): The condition that leads to the procedure is the AE. Situations in which an untoward medical occurrence did not occur (e.g., hospitalization for elective surgery if planned before the signing the ICF, admissions for social reasons or for convenience).
2. Anticipated day-to-day fluctuations of pre-existing disease(s) or condition(s) present or detected at the start of the study that do not worsen.
3. A medication error (including intentional misuse, abuse, and overdose of the product) or use other than what is defined in the protocol is not considered an AE unless there is an untoward medical occurrence as a result of a medication error.
4. Cases of pregnancy that occur during maternal or paternal exposure to study intervention are to be reported within 24 hours of Investigator/site awareness. Data on fetal outcome and breastfeeding is collected for regulatory reporting and safety evaluation.
5. Any clinically significant abnormal laboratory findings or other abnormal safety assessments which are associated with the underlying disease, unless judged by the Investigator to be more severe than expected for the participant's condition.
6. The disease/disorder being studied or expected progression, signs, or symptoms of the disease/disorder being studied, unless more severe than expected for the participant's condition.
7. Situations in which an untoward medical occurrence did not occur (social and/or convenience admission to a hospital).
8. “Lack of efficacy” or “failure of expected pharmacological action” per se are not reported as an AE or SAE. Such instances are captured in the efficacy assessments.

However, the signs, symptoms, and/or clinical sequelae resulting from lack of efficacy are reported as AE or SAE if they fulfil the definition of an AE or SAE.

If an event is not an AE per the definition above, then it cannot be a Serious Adverse Event (SAE) even if serious conditions are met (e.g., hospitalization for signs/symptoms of the disease under study, death due to progression of disease). A SAE is defined as defined as any untoward medical occurrence that, at any dose:
1. Results in death
2. Is life-threatening. The term “life-threatening” in the definition of “serious” refers to an event in which the participant was at risk of death at the time of the event. It does not refer to an event, which hypothetically might have caused death, if it was more severe.
3. Requires inpatient hospitalization or prolongation of existing hospitalization. In general, hospitalization signifies that the participant has been detained (usually involving at least an overnight stay) at the hospital or emergency ward for observation and/or treatment that would not have been appropriate in the physician's office or outpatient setting. Complications that occur during hospitalization are AEs. If a complication prolongs hospitalization or fulfills any other serious criteria, the event is serious. When in doubt as to whether “hospitalization” occurred or was necessary, the AE should be considered serious. Hospitalization for elective treatment of a pre-existing condition that did not worsen from baseline is not considered an AE.
4. Results in persistent disability/incapacity. The term disability means a substantial disruption of a person's ability to conduct normal life functions. This definition is not intended to include experiences of relatively minor medical significance such as uncomplicated headache, nausea, vomiting, diarrhea, influenza, and accidental trauma (e.g., sprained ankle) which may interfere with or prevent everyday life functions but do not constitute a substantial disruption.
5. Is a congenital anomaly/birth defect.
6. Other situations: Medical or scientific judgment should be exercised in deciding whether SAE reporting is appropriate in other situations such as important medical events that may not be immediately life-threatening or result in death or hospitalization but may jeopardize the participant or may require medical or surgical intervention to prevent one of the other outcomes listed in the above definition. These events should usually be considered serious. Examples of such events include invasive or malignant cancers, intensive treatment in an emergency room or at home for allergic bronchospasm, blood dyscrasias or convulsions that do not result in hospitalization, or development of drug dependency or drug abuse.

A suspected unexpected serious adverse reaction (SUSAR) is defined as an event that is assessed as serious by the Investigator that is not listed in the appropriate Reference Safety Information (IB) and has been assessed that there is at least a reasonable possibility that the event is related to the investigational medicinal product by the Investigator. Suspected unexpected serious adverse reactions undergo expedited reporting to the national regulatory authorities, IRBs/IECs, and Investigators following local regulatory reporting requirements where applicable.

With respect to the recording of an AE and/or SAE, it is the responsibility of the Investigator to review all documentation (e.g., hospital progress notes, laboratory reports, and diagnostics reports) related to the event. The Investigator then records all relevant AE/SAE information in the eCRF. The Investigator attempts to establish a diagnosis of the event based on signs, symptoms, and/or other clinical information. Whenever possible, the diagnosis (not the individual signs/symptoms) is documented as the AE/SAE.

The Investigator makes an assessment of intensity for each AE and SAE reported during the study and assign it to one of the following categories from National Cancer Institute CTCAE v5.0, published 27 Nov. 2017: Grade 1: Mild (awareness of sign or symptom, but easily tolerated), Grade 2: Moderate (discomfort sufficient to cause interference with normal activities), Grade 3: Severe (incapacitating, with inability to perform normal activities), Grade 4: Life-threatening, and Grade 5: Fatal. An event is defined as “serious” when it meets at least one of the predefined outcomes as described in the definition of an SAE, not when it is rated as severe.

The Investigator is obligated to assess the relationship between the study intervention and each occurrence of each AE or SAE. An Investigator causality assessment must be provided for all AEs (both nonserious and serious). This assessment must be recorded in the eCRF and on any additional forms, as appropriate. The definitions for the causality assessments are as follows: (1) Not related: There is no reasonable possibility the study intervention caused the AE. The AE has a more likely alternative etiology; it may be due to underlying or concurrent illness, complications, concurrent treatments, or effects of another concurrent drug. The event does not follow a reasonable temporal relationship to administration of the study intervention.

(2) Related: There is a reasonable possibility the study intervention caused the AE. The AE has a temporal relationship to the administration of the study intervention. The event does not have a likely alternative etiology. The event corresponds with the known pharmaceutical profile of the study intervention. There is improvement on discontinuation and/or reappearance on rechallenge.

The Investigator uses clinical judgment to determine the relationship. Alternative causes, such as underlying disease(s), concomitant therapy, and other risk factors, as well as the temporal relationship of the event to study intervention administration are considered and investigated. The Investigator also consults the IB and/or Product Information, for marketed products, in his/her assessment. For each AE/SAE, the Investigator must document in the medical notes that he/she has reviewed the AE/SAE and has provided an assessment of causality. There may be situations in which an SAE has occurred, and the Investigator has minimal information to include in the initial report. The Investigator may change his/her opinion of causality in light of follow-up information and send an SAE follow-up report with the updated causality assessment. The causality assessment is one of the criteria used when determining regulatory reporting requirements.

### 9. Pharmacokinetics and Pharmacodynamics

Blood samples for determination of serum drug concentrations and serum free and total C5 are collected before and after administration of study intervention at the time points specified in the Schedule of Activities. Instructions for the collection and handling of biological samples is provided. The actual date and time (24-hour clock time) of each sample is recorded on the eCRF and the central laboratory requisition form. Samples collected for analyses of ravulizumab serum concentration may also be used to evaluate safety or efficacy aspects related to concerns arising during or after the study. Baseline and trough PK/PD blood samples are collected at pre-dose, within 90 minutes before administering study intervention at visits specified in the Schedule of Activities. The pre-dose blood sample can be drawn through the venous access created for the dose infusion, prior to administration of the dose. Post-dose PK/PD blood samples are collected post-dose, within 60 minutes after completing study intervention infusion. The post-dose blood samples are drawn from the participant's opposite, non-infused arm. PK/PD blood samples collected at the ET/EOS visit and at unscheduled visits are collected at any time. In the event of an unscheduled visit, a PK/PD blood sample can be collected anytime.

### 10. Biomarkers

Blood (serum and plasma) samples for biomarker research are collected from all participants at the time points specified in the Schedule of Activities.

Biomarkers to be measured may include, but are not limited to, assessments of the following: Complement pathway dysregulation (e.g., soluble C5b-9, etc.) and myositis-specific autoantibodies (e.g., antimelanoma differentiation-associated protein 5 [anti-MDA5], antinuclear matrix protein 2 (anti NXP2/MJ), anti synthetase/Jo 1, etc.)

Remaining samples from PK, PD, immunogenicity, and biomarker testing are stored for additional method developments of assays (e.g., prognostics and/or companion diagnostics related to the study intervention target, disease process, pathways associated with disease state, other complement related diseases, and/or mechanism of action of ravulizumab).

Samples are retained to enable further analysis on ravulizumab but no longer than 5 years after termination of the study or other period as per local requirements.

### 11. Immunogenicity Assessments

Antibodies to ravulizumab (i.e., ADAs) are evaluated in serum samples collected predose (90 minutes prior to the start of infusion of study intervention) from all participants according to the Schedule of Activities. Additionally, serum samples should also be collected at the final visit from participants who discontinued the study intervention or were withdrawn from the study.

Serum samples are screened for antibodies binding to ravulizumab and the titer of confirmed positive samples is reported. Other analyses may be performed to further characterize the immunogenicity of ravulizumab.

The detection and characterization of antibodies to ravulizumab is performed using a validated/qualified assay method. Antibodies may be further characterized and/or evaluated for their ability to neutralize the activity of the study intervention(s). Samples may be stored for a maximum duration according to local regulations following the last participant's last visit to enable further analysis of immune responses to ravulizumab.

### 12. Statistical Considerations

Part A is exploratory in nature, and there is no planned formal statistical hypothesis testing.

With respect to Part B, the primary hypothesis is that the ravulizumab is superior to the placebo group in the primary estimand. The hypothesis for assessing superiority with respect to the proportions of responders is:

$${H}_{0} : {p}_{r a v} - {p}_{p b o} \leq 0 ; {H}_{1} : {p}_{r a v} - {p}_{p b o} > 0 ,$$

Where pᵣₐᵥ and p_{pbo} are the proportions of responders for ravulizumab and placebo, respectively.

With respect to Part A, a total number of 48 participants are randomized into the study with a 2:1 (ravulizumab:placebo) allocation ratio to ensure approximately 80% power to detect a treatment difference of 35% in IMACS-TIS minimum response (IMACS-TIS≥20) at 1 sided Type 1 error of 0.1, assuming a placebo response rate of 40% and approximately 15% dropout rate. The sample size was calculated in EAST 6.5 using the 2-sample test for the difference of proportions with unpooled estimate of variance for the primary endpoint (IMACS-TIS≥20).

With respect to Part B, a total number of 132 participants are randomized into the study with a 1:1 (ravulizumab:placebo) allocation ratio to ensure approximately 90% power to detect a treatment difference of 30% in IMACS-TIS minimum response (IMACS-TIS≥20) at 1 sided Type 1 error of 0.025, assuming a placebo response rate of 40% and approximately 20% dropout rate. The sample size was calculated in EAST 6.5 using the 2-sample test for the difference of proportions with unpooled estimate of variance.

An unblinded sample size re-estimation is planned to possibly increase the sample size to a maximum of 174 participants in Part B.

For the purposes of analysis, the following analysis sets for each of Part A and Part B independently are defined in Table 8. Unless otherwise specified, the same definitions are used for the statistical analyses of Part A and Part B where applicable.

TABLE 8
Study ALXN1210-DM-310 Analysis Populations

| Population | Description |
|---|---|
| Randomized Set | All randomized participants grouped by randomized treatment |
| | group (for reporting disposition, demographics, and baseline |
| | characteristics). |
| Pharmacokinetics (PK) | All participants who receive at least 1 dose of study intervention |
| Analysis Set (PKAS) | and have at least 1 post-dose PK sample. |
| Pharmacodynamics (PD) | All participants who receive at least 1 dose of study intervention |
| Analysis Set (PDAS) | and who have evaluable free or total C5 data. |
| Full Analysis Set (FAS) | All randomized participants who receive at least 1 dose of study |
| | intervention. Participants are analyzed as randomized. |
| | The FAS is used for the analysis of efficacy data from the |
| | Randomized Controlled Period and is considered the primary |
| | analysis population. |
| Per Protocol Set (PPS) | All FAS participants without any major protocol deviations^{a} that |
| | could impact efficacy analyses. |
| | The PPS is used for sensitivity analyses of the primary and |
| | secondary efficacy endpoints. |
| Safety Set (SS) | All randomized participants who receive at least 1 dose of study |
| | intervention. Participants are analyzed according to the study |
| | intervention they actually received. |
| | The SS is used for the analysis of safety data. |
| Open Label Extension | All randomized participants who received at least 1 dose of |
| (OLE) Set | ravulizumab starting from Week 26 (Part A) and Week 50 (Part |
| | B) onward (for reporting all data from the OLE Period). |

^{a}Determination of applicable major protocol deviations for this purpose is made prior to database lock and study unblinding.

Statistical analyses include tabulations of summary data, inferential analyses, by participant listings, and figures. The summary statistics for continuous variables include, but are not be limited to, the number of participants, mean, standard deviation, minimum, median, and maximum. For categorical variables, frequencies and percentages are presented.

All efficacy analyses are based on the Full Analysis Set (FAS). Supplemental per protocol analyses for primary and secondary efficacy endpoints are performed based on the Per Protocol Set (PPS) in the same manner as done for the FAS. Safety analyses are performed on the Safety Set (SS).

The baseline value for analysis and reporting is based on the last non missing measurement on or prior to the first dose of study intervention unless stated otherwise.

Analyses are performed using the SAS® software Version 9.4 or higher.

With respect to Part A, all data collected during the first 26-week Randomized Controlled Period in Part A is analyzed independently of Part B and not be included in the formal hypothesis testing of Part B. In general, the statistical testing in Part A is exploratory in nature and performed with 2-sided Type 1 error of 0.2. For endpoints on which such exploratory hypothesis testing is performed, the 2-sided p-value, the estimated difference between ravulizumab and placebo, and the 2-sided 80% confidence interval (CI) of the treatment difference is presented.

For Part A, the comparison of the treatment groups for the primary endpoint is based on the FAS with Mantel-Haenszel test of the difference in 2 proportions and a 2-sided Type I error of 0.2. The estimated Mantel-Haenszel risk difference is summarized along with the 2-sided 80% CI using Mantel-Haenszel stratum weights (see Mantel N, Haenszel W., J. Natl. Cancer Inst. 1959; 22(4):719-748) and the Sato variance estimator (see Sato T., letter to the editor. Biometrics. 1989; 45:1323-1324).

All secondary efficacy endpoints are analyzed using the FAS. For the comparison of the treatment difference between two independent samples, a mixed effect repeated measures model is used for the continuous efficacy endpoints; the Mantel Haenszel test is used for binary efficacy endpoints; and Kaplan Meier method is used for the survival (time-to-event) efficacy endpoints.

With respect to Part B, all data collected during the first 50-week Randomized Controlled Period in Part B is analyzed independently of Part A. The formal statistical hypothesis testing in Part B is performed with 2-sided Type 1 error of 0.05.

The primary null hypothesis is that the effect of ravulizumab is no different than placebo in TIS20 response. The alternative hypothesis is that there is a treatment difference from placebo in favor of ravulizumab based on TIS20 response.

The comparison of the treatment groups for the primary endpoint is based on the FAS with 2-sided Mantel-Haenszel test of the difference in 2 proportions stratified by randomization stratification factors and a Type 1 error of 0.05. The 2-sided p-value, the estimated Mantel Haenszel risk difference is presented along with the 2-sided 95% CI using Mantel Haenszel stratum weights (Mantel, 1959) and the Sato variance estimator (Sato, 1989).

The study is considered to have met its primary efficacy objective if the null hypothesis is rejected at a 2-sided statistically significant level of 0.05 in favor of ravulizumab.

The following supportive analyses is performed for the primary efficacy endpoint to explore the robustness of the IMACS-TIS results from the primary efficacy analysis due to intercurrent events. Additional supportive analyses may also be performed using reasonable penalization strategies for data after intercurrent events.

In this analysis, data after any of the intercurrent events is assumed to follow the trajectory of the participants without intercurrent event from the control treatment group who continued to the next visit (control-based) using a multiple imputation approach. Imputation is performed on the IMACS-TIS data, and the binary outcome of response (TIS20) is derived from the imputed TIS data.

Tipping Point Analysis: In this sensitivity analysis of delta-adjusted stress testing method (tipping point analysis), data from participants after any intercurrent event is assumed to experience worsening for the remainder of the Randomized Controlled Period, compared with participants who continue the study to the next visit without any intercurrent event in the same treatment group, with the worsening defined by a prespecified adjustment (delta) in the TIS score. Since a high TIS score (0 100) indicates better improvement, the prespecified value of delta is a negative quantity. A two-way tipping point analysis via multiple imputations is performed by using different delta adjustment for each treatment group. The binary response is derived from the imputed TIS data and is analyzed using the same primary analysis method as for the primary endpoint.

For each combination of delta values for ravulizumab and placebo, the treatment effect is determined, and the value of the deltas for which the nominal 2-sided p-value crosses 0.05 is considered as the “tipping point”, i.e., the positive conclusion drawn from the primary analysis is reversed when participants drop out are assumed to experience this fixed worsening after the discontinuation.

A supportive analysis of the primary endpoint is also performed using the Treatment Policy Estimand strategy. In this analysis, the same analysis method is used for the binary outcome (TIS20) from participants without regard to experiencing any intercurrent event. Missing data due to early discontinuation is imputed in a way that is consistent with the Treatment Policy Estimand.

For the binary primary composite estimand (TIS20), participants with missing TIS data from early discontinuation after experiencing any intercurrent event are considered as treatment failure (non-responders). Missing TIS data from participants who discontinued early without experiencing any intercurrent event are imputed using a missing at random (MAR) strategy via multiple imputation, and assumed to follow the trajectory of the participants without intercurrent event from the same treatment group who continued to the next visit. The binary TIS response data is derived from the imputed TIS data and analyzed using the statistical method for the primary endpoint.

For the continuous key secondary endpoint (IMACS-TIS), missing TIS data from participants who discontinue early after experiencing any intercurrent event is imputed in a way that is consistent with the primary composite estimand with a missing not at random (MNAR) strategy via multiple imputation. Missing TIS data from participants who discontinued early without experiencing any intercurrent event is imputed using a missing at random (MAR) strategy via multiple imputation, and assumed to follow the trajectory of the participants without intercurrent event from the same treatment group who continued to the next visit. The continuous TIS data is analyzed using statistical methods described herein.

With respect to Part B, the comparison of the treatment groups for the mean IMACS-TIS at Week 50 of the Randomized Controlled Period is based on the FAS. To be consistent with the primary composite estimand, a control-based multiple imputation strategy is adopted. In this approach, data from participants after experiencing any of the intercurrent events is assumed to follow the trajectory of the participants without intercurrent events from the control treatment group who continued to the next visit using a multiple imputation approach.

A mixed effect repeated measures model is used for the statistical analysis of the imputed TIS data. The model includes response variables at each pre specified time point as the dependent variable, fixed categorical effects of treatment, study visit, and treatment-by-study visit interaction, and region, as well as important baseline covariates. The treatment effect is evaluated via contrast for the treatment-by-visit term at Week 50. An unstructured covariance matrix is used to model the correlations among repeated measurements within each participant. Other covariance structures are implemented if a convergence issue occurs.

The 2-sided p-value, estimated least square means difference between ravulizumab and placebo along with the 2-sided 95% CI of the difference from the model are presented.

The survival (time-to-event) endpoints are analyzed using the method of Kaplan Meier and compared using a log rank test stratified by region and cutaneous manifestations. Hazard ratio and risk reduction are summarized from a Cox proportional hazards model stratified by region and cutaneous manifestations. CIs (95%) are presented for the survival estimate based on the complementary log-log transformation. Kaplan-Meier curves for both treatment groups are produced.

The final primary hypothesis for the primary endpoint are tested at a 2 sided Type 1 error of 0.05 with a final test statistics adjusted for the planned adaptive sample size re-estimation to control the overall Type I error for Part B. Hypothesis testing associated with the key secondary endpoints proceeds only if the null hypothesis associated with the primary endpoint is rejected.

The safety and tolerability of ravulizumab is assessed based on AE reports, clinical laboratory findings, ECGs, and vital signs findings. Safety analyses are performed on the SS population.

Analysis and reporting for AEs is based on treatment-emergent adverse events (TEAEs), including treatment-emergent serious adverse events (TESAEs) and TEAE leading to drug discontinuation, defined as an AE with onset on or after first dose of study intervention in the Randomized Controlled Period. TEAEs and TESAEs and TEAE leading to drug discontinuation is summarized by Medical Dictionary for Regulatory Activities (MedDRA) System Organ Class (SOC) and Preferred Term, by severity, and by relationship to the study intervention. Participant-years adjusted event rates are generated to characterize long term safety profile.

Laboratory measurements as well as their changes from Baseline at each visit and shift from baseline, if applicable, are summarized descriptively. Significant findings related to ECG and vital signs are also summarized using descriptive analyses.

Individual serum concentration data for all participants who receive at least 1 dose of study intervention and have at least 1 post-dose PK sample are used to derive PK parameters for ravulizumab.

Graphs of mean serum concentration-time profiles are constructed. Graphs of serum concentration-time profiles for individual participants may also be provided.

Descriptive statistics are calculated for serum concentration data at each sampling time, as appropriate. Assessment of population-PK may be considered using data from this study or in combination with data from other studies.

Descriptive statistics are presented for all ravulizumab PD endpoints at each sampling time. The PD effects of ravulizumab administered IV is evaluated by assessing the absolute values and changes and percent changes from baseline in free and total C5 serum concentrations over time, as appropriate. Boxplots of absolute values of free and total C5 serum concentrations by study visit are constructed. Assessments of ravulizumab PK/PD relationships may be explored using data from this study or in combination with data from other studies.

### Analysis of Immunogenicity is Based on the SS.

The presence of ADAs to ravulizumab in serum is assessed over the study duration. Further characterization of antibody responses may be conducted as appropriate, including neutralizing antibodies and the titer of confirmed positive samples. Immunogenicity results are analyzed by summarizing the number and percentage of participants who develop confirmed positive ADAs. The association of ADAs with ravulizumab concentration, PD parameters, efficacy, and TEAEs may be explored as appropriate.

Analyses of exploratory biomarkers are described in a separate analysis plan.

With respect to Part A, an interim analysis may be performed for administrative purposes. The interim analysis is conducted with discretion when a total of approximately 24 (50%) participants reach the pre-dose Week 26 Visit or have discontinued prematurely. The comparative primary and key efficacy as well as safety data are assessed at this interim analysis. There is no plan to alter the Part A study design therefore no Type 1 error adjustment is needed for the final statistical analysis. The interim analysis is conducted by an independent DMC to maintain the blinding.

The primary efficacy endpoint as well as the safety data is assessed at this interim analysis. If futility criteria are met or if the benefit/risk is not considered favorable, further enrollment can be stopped.

With respect to Part B, one interim analysis may be conducted for sample size re-estimation. The interim analysis for the unblinded sample re-estimation is conducted with discretion when approximately 30% of the participants have completed the pre-dose Week 50 visit or have discontinued prematurely in Part B. The sample size is increased to a maximum of 174 participants using a conditional power approach. A combination test of the weighted stage wise statistics is used for the final statistical analysis to control the Type 1 error due to the planned adaptive sample size increase. The interim analysis is performed by an independent DMC to maintain study integrity and blinding.

SEQUENCE SUMMARY

SEQ ID NO: 1
GYIFSNYWIQ
SEQ ID NO: 2
EILPGSGSTEYTENFKD
SEQ ID NO: 3
YFFGSSPNWYFDV
SEQ ID NO: 4
GASENIYGALN
SEQ ID NO: 5
GATNLAD
SEQ ID NO: 6
QNVLNTPLT
SEQ ID NO: 7
QVQLVQSGAE VKKPGASVKV SCKASGYIFS NYWIQWVRQA PGQGLEWMGE ILPGSGSTEY TENFKDRVTM
TRDTSTSTVY MELSSLRSED TAVYYCARYF FGSSPNWYFD VWGQGTLVTV SS
SEQ ID NO: 8
DIQMTQSPSS LSASVGDRVT ITCGASENIY GALNWYQQKP GKAPKLLIYG ATNLADGVPS RFSGSGSGTD
FTLTISSLQP EDFATYYCQN VLNTPLTFGQ GTKVEIK
SEQ ID NO: 9
ASTKGPSVFP LAPCSRSTSE STAALGCLVK DYFPEPVTVS WNSGALTSGV HTFPAVLQSS GLYSLSSVVT
VPSSNFGTQT YTCNVDHKPS NTKVDKTVER KCCVECPPCP APPVAGPSVF LFPPKPKDTL MISRTPEVTC
VVVDVSQEDP EVQFNWYVDG VEVHNAKTKP REEQFNSTYR VVSVLTVLHQ DWLNGKEYKC KVSNKGLPSS
IEKTISKAKG QPREPQVYTL PPSQEEMTKN QVSLTCLVKG FYPSDIAVEW ESNGQPENNY KTTPPVLDSD
GSFFLYSRLT VDKSRWQEGN VFSCSVMHEA LHNHYTQKSL SLSLGK
SEQ ID NO: 10
QVQLVQSGAE VKKPGASVKV SCKASGYIFS NYWIQWVRQA PGQGLEWMGE ILPGSGSTEY TENFKDRVTM
TRDTSTSTVY MELSSLRSED TAVYYCARYF FGSSPNWYFD VWGQGTLVTV SSASTKGPSV FPLAPCSRST
SESTAALGCL VKDYFPEPVT VSWNSGALTS GVHTFPAVLQ SSGLYSLSSV VTVPSSNFGT QTYTCNVDHK
PSNTKVDKTV ERKCCVECPP CPAPPVAGPS VFLFPPKPKD TLMISRTPEV TCVVVDVSQE DPEVQFNWYV
DGVEVHNAKT KPREEQFNST YRVVSVLTVL HQDWLNGKEY KCKVSNKGLP SSIEKTISKA KGQPREPQVY
TLPPSQEEMT NQVSLTCLVK GFYPSDIAVE WESNGQPENN YKTTPPVLDS DGSFFLYSRL TVDKSRWQEG
NVFSCSVMHE ALHNHYTQKS LSLSLGK
SEQ ID NO: 11
DIQMTQSPSS LSASVGDRVT ITCGASENIY GALNWYQQKP GKAPKLLIYG ATNLADGVPS RFSGSGSGTD
FTLTISSLQP EDFATYYCQN VLNTPLTFGQ GTKVEIKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 12
QVQLVQSGAE VKKPGASVKV SCKASGHIFS NYWIQWVRQA PGQGLEWMGE ILPGSGHTEY TENFKDRVTM
TRDTSTSTVY MELSSLRSED TAVYYCARYF FGSSPNWYFD VWGQGTLVTV SS
SEQ ID NO: 13
ASTKGPSVFP LAPCSRSTSE STAALGCLVK DYFPEPVTVS WNSGALTSGV HTFPAVLQSS GLYSLSSVVT
VPSSNFGTQT YTCNVDHKPS NTKVDKTVER KCCVECPPCP APPVAGPSVF LFPPKPKDTL MISRTPEVTC
VVVDVSQEDP EVQFNWYVDG VEVHNAKTKP REEQFNSTYR VVSVLTVLHQ DWLNGKEYKC KVSNKGLPSS
IEKTISKAKG QPREPQVYTL PPSQEEMTKN QVSLTCLVKG FYPSDIAVEW ESNGQPENNY KTTPPVLDSD
GSFFLYSRLT VDKSRWQEGN VFSCSVLHEA LHSHYTQKSL SLSLGK
SEQ ID NO: 14
QVQLVQSGAE VKKPGASVKV SCKASGHIFS NYWIQWVRQA PGQGLEWMGE ILPGSGHTEY TENFKDRVTM
TRDTSTSTVY MELSSLRSED TAVYYCARYF FGSSPNWYFD VWGQGTLVTV SSASTKGPSV FPLAPCSRST
SESTAALGCL VKDYFPEPVT VSWNSGALTS GVHTFPAVLQ SSGLYSLSSV VTVPSSNFGT QTYTCNVDHK
PSNTKVDKTV ERKCCVECPP CPAPPVAGPS VFLFPPKPKD TLMISRTPEV TCVVVDVSQE DPEVQFNWYV
DGVEVHNAKT KPREEQFNST YRVVSVLTVL HQDWLNGKEY KCKVSNKGLP SSIEKTISKA KGQPREPQVY
TLPPSQEEMT KNQVSLTCLV KGFYPSDIAV EWESNGQPEN NYKTTPPVLD SDGSFFLYSR LTVDKSRWQE
GNVFSCSVLH EALHSHYTQK SLSLSLGK
SEQ ID NO: 15
ASTKGPSVFP LAPCSRSTSE STAALGCLVK DYFPEPVTVS WNSGALTSGV HTFPAVLQSS GLYSLSSVVT
VTSSNFGTQT YTCNVDHKPS NTKVDKTVER KCCVECPPCP APPVAGPSVF LFPPKPKDTL YITREPEVTC
VVVDVSHEDP EVQFNWYVDG MEVHNAKTKP REEQFNSTFR VVSVLTVVHQ DWLNGKEYKC KVSNKGLPAP
IEKTISKTKG QPREPQVYTL PPSREEMTKN QVSLTCLVKG FYPSDIAVEW ESNGQPENNY KTTPPMLDSD
GSFFLYSKLT VDKSRWQQGN VFSCSVMHEA LHNHYTQKSL SLSPGK
SEQ ID NO: 16
QVQLVQSGAE VKKPGASVKV SCKASGYIFS NYWIQWVRQA PGQGLEWMGE ILPGSGSTEY TENFKDRVTM
TRDTSTSTVY MELSSLRSED TAVYYCARYF FGSSPNWYFD VWGQGTLVTV SSASTKGPSV FPLAPCSRST
SESTAALGCL VKDYFPEPVT VSWNSGALTS GVHTFPAVLQ SSGLYSLSSV VTVTSSNEGT QTYTCNVDHK
PSNTKVDKTV ERKCCVECPP CPAPPVAGPS VFLFPPKPKD TLYITREPEV TCVVVDVSHE DPEVQFNWYV
DGMEVHNAKT KPREEQFNST FRVVSVLTVV HQDWLNGKEY KCKVSNKGLP APIEKTISKT KGQPREPQVY
TLPPSREEMT KNQVSLTCLV KGFYPSDIAV EWESNGQPEN NYKTTPPMLD SDGSFFLYSK LTVDKSRWQQ
GNVFSCSVMH EALHNHYTQK SLSLSPGK
SEQ ID NO: 17
GASENIYHALN
SEQ ID NO: 18
EILPGSGHTEYTENFKD
SEQ ID NO: 19
GHIFSNYWIQ
SEQ ID NO: 20
QVQLVQSGAE VKKPGASVKV SCKASGHIFS NYWIQWVRQA PGQGLEWMGE ILPGSGHTEY TENFKDRVTM
TRDTSTSTVY MELSSLRSED TAVYYCARYF FGSSPNWYFD VWGQGTLVTV SSASTKGPSV FPLAPCSRST
SESTAALGCL VKDYFPEPVT VSWNSGALTS GVHTFPAVLQ SSGLYSLSSV VTVPSSNFGT QTYTCNVDHK
PSNTKVDKTV ERKCCVECPP CPAPPVAGPS VFLFPPKPKD TLMISRTPEV TCVVVDVSQE DPEVQFNWYV
DGVEVHNAKT KPREEQFNST YRVVSVLTVL HQDWLNGKEY KCKVSNKGLP SSIEKTISKA KGQPREPQVY
TLPPSQEEMT KNQVSLTCLV KGFYPSDIAV EWESNGQPEN NYKTTPPVLD SDGSFFLYSR LTVDKSRWQE
GNVFSCSVMH EALHNHYTQK SLSLSLGK
SEQ ID NO: 21
SYAIS
SEQ ID NO: 22
GIGPFFGTANYAQKFQG
SEQ ID NO: 23
DTPYFDY
SEQ ID NO: 24
SGDSIPNYYVY
SEQ ID NO: 25
DDSNRPS
SEQ ID NO: 26
QSFDSSLNAEV
SEQ ID NO: 27
QVQLVQSGAE VKKPGSSVKV SCKASGGTFS SYAISVWRQA PGQGLEWMGG IGPFFGTANY AQKFQGRVTI
TADESTSTAY MELSSLRSED TAVYYCARDT PYFDYWGQGT LVTVSS
SEQ ID NO: 28
DIELTQPPSV SVAPGQTARI SCSGDSIPNY YVYWYQQKPG QAPVLVIYDD SNRPSGIPER FSGSNSGNTA
TLTISGTQAE DEADYYCQSF DSSLNAEVFG GGTKLTVL
SEQ ID NO: 29
SSYYVA
SEQ ID NO: 30
AIYTGSGATYKASWAKG
SEQ ID NO: 31
DGGYDYPTHAMHY
SEQ ID NO: 32
QASQNIGSSLA
SEQ ID NO: 33
GASKTHS
SEQ ID NO: 34
QSTKVGSSYGNH
SEQ ID NO: 35
QVQLVESGGG LVQPGGSLRL SCAASGFTSH SSYYVAWVRQ APGKGLEWVG AIYTGSGATY KASWAKGRFT
ISKDTSKNQV VLTMTNMDPV DTATYYCASD GGYDYPTHAM HYWGQGTLVT VSS
SEQ ID NO: 36
DVVMTQSPSS LSASVGDRVT ITCQASQNIG SSLAWYQQKP GQAPRLLIYG ASKTHSGVPS RFSGSGSGTD
FTLTISSLQP EDVATYYCQS TKVGSSYGNH FGGGTKVEIK
SEQ ID NO: 37
QVQLVESGGG LVQPGRSLRL SCAASGFTVH SSYYMAWVRQ APGKGLEWVG AIFTGSGAEY KAEWAKGRVT
ISKDTSKNQV VLTMTNMDPV DTATYYCASD AGYDYPTHAM HYWGQGTLVT VSSASTKGPS VFPLAPSSKS
TSGGTAALGC LVKDYFPEPV TVSWNSGALT SGVHTFPAVL QSSGLYSLSS VVTVPSSSLG TQTYICNVNH
KPSNTKVDKK VEPKSCDKTH TCPPCPAPEL RRGPKVFLFP PKPKDTLMIS RTPEVTCVVV DVSHEDPEVK
FNWYVDGVEV HNAKTKPREE QYNSTYRVVS VLTVLHQDWL NGKEYKCKVS NKGLPSSIEK TISKAKGQPR
EPQVYTLPPS REEMTKNQVS LTCLVKGFYP SDIAVEWESN GQPENNYKTT PPVLDSDGSF FLYSKLTVDK
SRWQQGNVFS CSVLHEALHA HYTRKELSLS P
SEQ ID NO: 38
DIQMTQSPSS LSASVGDRVT ITCRASQGIS SSLAWYQQKP GKAPKLLIYG ASETESGVPS RFSGSGSGTD
FTLTISSLQP EDFATYYCQN TKVGSSYGNT FGGGTKVEIK RFSGSGSGTD FPPSDEQLKS GTASVVCLLN
NFYPREAKVQ WKVDNALQSG RTVAAPSVFI SKDSTYSLSS TLTLSKADYE KHKVYACEVT HQGLSSPVTK
NSQESVTEQD
SEQ ID NO: 39
QVQLQESGPGLVKPSETLSLTCTVSGDSVSSSYWTWIRQPPGKGLEWIGYIYYSGSSNYNPSLKSRATISVDTSKN
QFSLKLSSVTAADTAVYYCAREGNVDTTMIFDYWGQGTLVTVSS
SEQ ID NO: 40
AIQMTQSPSSLSASVGDRVTITCRASQGIRNDLGWYQQKPGKAPKLLIYAASSLQSGVPSRFAGRGSGTDFTLTIS
SLQPEDFATYYCLQDENYPWTFGQGTKVEIK
SEQ ID NO: 41
QVQLQESGPGLVKPSETLSLTCTVSGDSVSSSYWTWIRQPPGKGLEWIGYIYYSGSSNYNPSLKSRATISVDTSKN
QFSLKLSSVTAADTAVYYCAREGNVDTTMIFDYWGQGTLVTVSSASTKGPSVFPLAPCSRSTSESTAALGCLVKDY
FPEPVTVSWNSGALTSGVHTFPAVLQSSGLYSLSSVVTVPSSSLGTKTYTCNVDHKPSNTKVDKRVESKYGPPCPP
CPAPEFLGGPSVFLFPPKPKDTLMISRTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHYTQKSLSLSLGK
SEQ ID NO: 42
AIQMTQSPSSLSASVGDRVTITCRASQGIRNDLGWYQQKPGKAPKLLIYAASSLQSGVPSRFAGRGSGTDFTLTIS
SLQPEDFATYYCLQDFNYPWTFGQGTKVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSSPVTKSENRGEC

What is claimed is:

1. A method of treating a human patient with dermatomyositis (DM), the method comprising administering to the patient an effective amount of an anti-C5 antibody, or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody or antigen binding fragment thereof, is administered:
(a) at a loading dose of 900 mg, followed by a maintenance dose of 2100 mg two weeks later and then once every eight weeks thereafter to a patient weighing <30 kg;
(b) at a loading dose of 1200 mg, followed by a maintenance dose of 2700 mg two weeks later and then once every eight weeks thereafter to a patient weighing ≥30to <40 kg;
(c) at a loading dose of 2400 mg, followed by a maintenance dose of 3000 mg two weeks later and then once every eight weeks thereafter to a patient weighing ≥40 to <60 kg;
(d) at a loading dose of 2700 mg, followed by a maintenance dose of 3300 mg two weeks later and then once every eight weeks thereafter to a patient weighing ≥60 to <100 kg; or
(e) at a loading dose of 3000 mg, followed by a maintenance dose of 3600 mg two weeks later and then once every eight weeks thereafter to a patient weighing ≥100.

2. The method of claim 1, wherein the anti C5 antibody, or antigen binding fragment thereof, is administered:
(a) at a loading dose of 900 mg on Day 1, followed by a maintenance dose of 2100 mg on Day 15 and then once every eight weeks thereafter to a patient weighing <30 kg;
(b) at a loading dose of 1200 mg on Day 1, followed by a maintenance dose of 2700 mg on Day 15 and then once every eight weeks thereafter to a patient weighing ≥30 to <40 kg;
(c) at a loading dose of 2400 mg on Day 1, followed by a maintenance dose of 3000 mg on Day 15 and then once every eight weeks thereafter to a patient weighing ≥40 to <60 kg;
(d) at a loading dose of 2700 mg on Day 1, followed by a maintenance dose of 3300 mg on Day 15 and then once every eight weeks thereafter to a patient weighing ≥60 to <100 kg; or
(e) at a loading dose of 3000 mg on Day 1, followed by a maintenance dose of 3600 mg on Day 15 and then once every eight weeks thereafter to a patient weighing ≥100.

3. The method of claim 1 or 2, wherein the anti-C5 antibody, or antigen binding fragment thereof, further comprises a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering.

4. The method of any one of the preceding claims, wherein the anti-C5 antibody comprises a heavy chain variable region set forth in SEQ ID NO:12 and a light chain variable region set forth in SEQ ID NO:8.

5. The method of any one of the preceding claims, wherein the anti-C5 antibody further comprises a heavy chain constant region set forth in SEQ ID NO:13.

6. The method of any one of the preceding claims, wherein the antibody comprises a heavy chain polypeptide comprising the amino acid sequence set forth in SEQ ID NO:14 and a light chain polypeptide comprising the amino acid sequence set forth in SEQ ID NO:11.

7. The method of any one of the preceding claims, wherein the anti-C5 antibody binds to human C5 at pH 7.4 and 25° C. with an affinity dissociation constant (K_{D}) that is in the range 0.1 nM≤K_{D}≤1 nM (e.g., about 0.5 nM).

8. The method of any one of the preceding claims, wherein the anti-C5 antibody binds to human C5 at pH 6.0 and 25° C. with a K_{D}≥10 nM (e.g., about 22 nM).

9. The method of any one of the preceding claims, wherein the anti-C5 antibody is administered to a patient weighing <30 kg at a loading dose of 900 mg, followed by a maintenance dose of 2100 mg two weeks later and then once every eight weeks thereafter.

10. The method of any one of claims 1-8, wherein the anti-C5 antibody is administered to a patient weighing ≥30 to <40 kg at a loading dose of 1200 mg, followed by a maintenance dose of 2700 mg two weeks later and then once every eight weeks thereafter.

11. The method of any one of claims 1-8, wherein the anti-C5 antibody is administered to a patient weighing ≥40 to <60 kg at a loading dose of 2400 mg, followed by a maintenance dose of 3000 mg two weeks later and then once every eight weeks thereafter.

12. The method of any one of claims 1-8, wherein the anti-C5 antibody is administered to a patient weighing ≥60 to <100 kg at a loading dose of 2700 mg, followed by a maintenance dose of 3300 mg two weeks later and then once every eight weeks thereafter.

13. The method of any one of claims 1-8, wherein the anti-C5 antibody is administered to a patient weighing ≥100 kg at a loading dose of 3000 mg on Day 1, followed by a maintenance dose of 3600 mg on Day 15 and then once every eight weeks thereafter.

14. The method of any one of the preceding claims, wherein the anti-C5 antibody is administered to a patient weighing <30 kg at a loading dose of 900 mg on Day 1, followed by a maintenance dose of 2100 mg on Day 15 and then once every eight weeks thereafter.

15. The method of any one of claims 1-8, wherein the anti-C5 antibody is administered to a patient weighing ≥30 to <40 kg at a loading dose of 1200 mg on Day 1, followed by a maintenance dose of 2700 mg on Day 15 and then once every eight weeks thereafter.

16. The method of any one of claims 1-8, wherein the anti-C5 antibody is administered to a patient weighing ≥40 to <60 kg at a loading dose of 2400 mg on Day 1, followed by a maintenance dose of 3000 mg on Day 15 and then once every eight weeks thereafter.

17. The method of any one of claims 1-8, wherein the anti-C5 antibody is administered to a patient weighing ≥60 to <100 kg at a loading dose of 2700 mg on Day 1, followed by a maintenance dose of 3300 mg on Day 15 and then and then once every eight weeks thereafter.

18. The method of any one of claims 1-8, wherein the anti-C5 antibody is administered to a patient weighing ≥100 kg at a loading dose of 3000 mg, followed by a maintenance dose of 3600 mg on Day 15 and then once every eight weeks thereafter.

19. The method of any one of the preceding claims, wherein the treatment maintains a serum trough concentration of the anti-C5 antibody of 100 μg/mL or greater.

20. The method of any one of the preceding claims, wherein the treatment maintains a serum trough concentration of the anti-C5 antibody of 200 μg/mL or greater.

21. The method of any one of the preceding claims, wherein the anti-C5 antibody is administered intravenously.

22. The method of any one of the preceding claims, wherein the treatment results in a shift towards normal levels of soluble C5b-9.

23. The method of any one of the preceding claims, wherein the treatment results in a shift towards normal levels of myositis-specific autoantibodies.

24. The method of claim 23, wherein the autoantibodies are anti-melanoma differentiation-associated protein 5 antibodies (anti-MDA5 antibodies), anti-nuclear matrix protein 2 antibodies (anti-NXP2/MJ antibodies), or anti-synthetase/Jo 1 antibodies.

25. The method of any one of the preceding claims, wherein the treatment results in a shift towards normal levels of muscle enzymes.

26. The method of any one of the preceding claims, wherein the treatment results in an improvement in the patient as assessed by a International Myositis Assessment and Clinical Studies Total Improvement Scale (IMACS-TIS), compared to baseline.

27. The method of any one of the preceding claims, wherein the treatment results in an at least >20-point Total Improvement Score (TIS) as assessed by an IMACS-TIS score, compared to baseline.

28. The method of any one of the preceding claims, wherein the treatment results in an at least >40-point TIS as assessed by an IMACS-TIS, compared to baseline.

29. The method of any one of the preceding claims, wherein the treatment results in an at least >60-point TIS as assessed by an IMACS-TIS, compared to baseline.

30. The method of any one of the preceding claims, wherein the treatment results in an improvement in the patient as assessed by a Cutaneous Dermatomyositis Disease Area and Severity Index (CDASI), compared to baseline.

31. The method of any one of the preceding claims, wherein the treatment results in a >7-point improvement in the patient as assessed by a CDASI, compared to baseline.

32. The method of any one of the preceding claims, wherein the treatment results in an improvement in the patient as assessed by a European Quality of Life Health 5-item questionnaire dimensions 5 level (EQ-5D-5L) score, compared to baseline.

33. The method of any one of the preceding claims, wherein the treatment results in an improvement in the patient as assessed by a Patient-reported Outcomes Measurement Information System (PROMIS), compared to baseline.

34. The method of any one of the preceding claims, wherein the treatment results in an improvement in the patient as assessed by a Short Form Health Survey (36 questions version) (SF 36), compared to baseline.

35. The method of any one of the preceding claims, wherein the treatment results in an improvement in the patient as assessed by a Dermatomyositis Disease Symptom Questionnaire (DM-DSQ), compared to baseline.

36. The method of any one of the preceding claims, wherein the treatment results in an improvement in the patient as assessed by a 30-Second Chair Stand Test (30s CST), compared to baseline.

37. The method of any one of the preceding claims, wherein the treatment results in a decrease in the patient's detectable rash as assessed by photographic analysis, compared to baseline.

38. The method of any one of the preceding claims, wherein the treatment results in an improvement in the patient as assessed by a 5D-itch scale, compared to baseline.

39. The method of any one of the preceding claims, wherein the treatment results in an improvement in the patient as assessed by a handheld dynamometry performance analysis, compared to baseline.

40. The method of any one of the preceding claims, wherein the treatment results in an improvement in the patient as assessed by a Functional Assessment of Chronic Therapy (FACIT)-Fatigue score, compared to baseline.

41. The method of any one of the preceding claims, wherein the treatment results in an improvement in the patient as assessed by a manual muscle testing subset of 8 muscles (MMT8), compared to baseline.

42. The method of any one of the preceding claims, wherein the treatment results in an improvement in the patient as assessed by a Myositis Disease Activity Assessment Tool (MDAAT), compared to baseline.

43. The method of any one of the preceding claims, wherein the treatment results in an improvement in the patient as assessed by a Physician Global Activity Assessment, compared to baseline.

44. The method of any one of the preceding claims, wherein the treatment results in an improvement in the patient as assessed by a Patient Global Activity Assessment, compared to baseline.

45. The method of any one of the preceding claims, wherein the treatment results in an improvement in the patient as assessed by a Health Assessment Questionnaire (HAQ), compared to baseline.

46. The method of any one of the preceding claims, wherein the treatment results in an improvement in the patient as assessed by a Cutaneous Dermatomyositis Activity Physician's Global Assessment (CD-IGA), compared to baseline.

47. The method of any one of the preceding claims, wherein the treatment results in a reduction or cessation in the patient's rash and/or muscle weakness.

48. The method of any one of the preceding claims, wherein the patient has an MMT-8 of ≥142/150 and two or more of the following prior to treatment:
f) a Patient Global Activity assessment of ≥2.0 cm on a 10 cm VAS
g) a Physician Global Activity assessment of ≥2.0 cm on a 10 cm VAS
h) a HAQ disability index with ≥0.25
i) elevation of at least one muscle enzyme at ≥1.3 times the upper limit of normal (ULN); and/or
j) a global extramuscular disease activity score with ≥2.0 cm on a 10 cm visual analog scale (VAS).

49. The method of claim 48, wherein the global extramuscular disease activity score is based on assessments of activity scores on the constitutional, cutaneous, skeletal, gastrointestinal, pulmonary, and cardiac scales of the MDAAT.

50. The method of any one of the preceding claims, wherein the patient has one or more of the following prior to treatment:
f) muscle or skin biopsy with evidence of active pathological findings of DM within last 6 months prior to or upon initiating treatment;
g) electromyography evidence of active myositis within the last 6 months prior to or upon initiating treatment;
h) magnetic resonance imaging (MRI) muscle evidence of active myositis within the last 6 months prior to or upon initiating treatment;
i) at least one muscle enzyme in an IMACS panel ≥2 times ULN prior to or upon initiating treatment; and/or
j) active DM skin rash characterized by inflammatory changes (CDASI Activity Score ≥7) prior to or upon initiating treatment.

51. The method of claim 48 or 50, wherein the at least one muscle enzyme is selected from the group consisting of creatine kinase (CK), aldolase, lactate dehydrogenase (LDH), alanine aminotransferase (ALT), and aspartate aminotransferase (AST).

52. The method of any one of the proceeding claims, wherein the treatment further comprises administering one or more of the following:
a) Azathioprine;
b) Cyclosporine;
c) Glucocorticoid;
d) Intramuscular glucocorticoids;
e) Hydroxychloroquine;
f) Leflunomide;
g) Methotrexate;
h) Mycophenolate mofetil/mycophenolic acid; and/or
i) Sulfasalazine.

53. The method of any one of the proceeding claims, wherein the treatment further comprises administering one or more of the following:
a) antihistamines;
b) ibuprofen;
c) acetaminophen;
d) anti-pruritics;
e) topical steroids;
f) vitamin B12;
g) vitamin E,
h) creatine,
i) coenzyme Q10, and/or
j) biotin supplements.

54. The method of any one of the proceeding claims, wherein the patient has not previously taken or is not taking any of the following upon initiating or during treatment:
a) intravenous immunoglobulin (IVIg);
b) subcutaneous immunoglobulin (SCIg);
c) IV glucocorticoids;
d) Corticotropin injection;
e) Cyclophosphamide;
f) Rituximab;
g) Infliximab;
h) Adalimumab;
i) Etanercept;
j) Tofacitinib;
k) Ruxolitinib; or
1) Anakinra.

55. The method of any one of the preceding claims, wherein the treatment results in terminal complement inhibition.

56. The method of any one of the preceding claims, wherein the treatment results in a reduction in adverse events.

57. The method of any one of the preceding claims, wherein the human patient is an adult patient.

58. A kit for treating DM in a human patient, the kit comprising:
(a) a dose of an anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and
(b) instructions for using the anti-C5 antibody, or antigen binding fragment thereof, in the method of any one of the preceding claims

59. An anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8, wherein the anti-C5 antibody, or antigen binding fragment thereof, is administered:
(a) at a loading dose of 900 mg, followed by a maintenance dose of 2100 mg two weeks later and then once every eight weeks thereafter to a patient weighing <30 kg;
(b) at a loading dose of 1200 mg, followed by a maintenance dose of 2700 mg two weeks later and then once every eight weeks thereafter to a patient weighing ≥30 to <40 kg;
(c) at a loading dose of 2400 mg, followed by a maintenance dose of 3000 mg two weeks later and then once every eight weeks thereafter to a patient weighing ≥40 to <60 kg;
(d) at a loading dose of 2700 mg, followed by a maintenance dose of 3300 mg two weeks later and then once every eight weeks thereafter to a patient weighing ≥60 to <100 kg; or
(e) at a loading dose of 3000 mg, followed by a maintenance dose of 3600 mg two weeks later and then once every eight weeks thereafter to a patient weighing ≥100.

60. An anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8, wherein the anti-C5 antibody, or antigen binding fragment thereof, is administered:
(a) at a loading dose of 900 mg on Day 1, followed by a maintenance dose of 2100 mg on Day 15 and then once every eight weeks thereafter to a patient weighing <30 kg;
(b) at a loading dose of 1200 mg on Day 1, followed by a maintenance dose of 2700 mg on Day 15 and then once every eight weeks thereafter to a patient weighing ≥30 to <40 kg;
(c) at a loading dose of 2400 mg on Day 1, followed by a maintenance dose of 3000 mg on Day 15 and then once every eight weeks thereafter to a patient weighing ≥40 to <60 kg;
(d) at a loading dose of 2700 mg on Day 1, followed by a maintenance dose of 3300 mg on Day 15 and then once every eight weeks thereafter to a patient weighing ≥60 to <100 kg; or
(e) at a loading dose of 3000 mg on Day 1, followed by a maintenance dose of 3600 mg on Day 15 and then once every eight weeks thereafter to a patient weighing ≥100.

61. The method of any one of the preceding claims, wherein the antibody is determined to be safe, tolerable, efficacious and sufficiently non-immunogenic after multiple IV doses in human patients.


SEQUENCE LISTING (42 sequences)

SEQ ID NO: 1 - protein, 10 aa, Artificial Sequence
  Description of Artificial Sequence Synthetic peptide
GYIFSNYWIQ

SEQ ID NO: 2 - protein, 17 aa, Artificial Sequence
  Description of Artificial Sequence Synthetic peptide
EILPGSGSTEYTENFKD

SEQ ID NO: 3 - protein, 13 aa, Artificial Sequence
  Description of Artificial Sequence Synthetic peptide
YFFGSSPNWYFDV

SEQ ID NO: 4 - protein, 11 aa, Artificial Sequence
  Description of Artificial Sequence Synthetic peptide
GASENIYGALN

SEQ ID NO: 5 - protein, 7 aa, Artificial Sequence
  Description of Artificial Sequence Synthetic peptide
GATNLAD

SEQ ID NO: 6 - protein, 9 aa, Artificial Sequence
  Description of Artificial Sequence Synthetic peptide
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 447 aa, Artificial Sequence
  Description of Artificial Sequence Synthetic polypeptide
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  Description of Artificial Sequence Synthetic polypeptide
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  Description of Artificial Sequence Synthetic polypeptide
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  Description of Artificial Sequence Synthetic peptide
GASENIYHALN

SEQ ID NO: 18 - protein, 17 aa, Artificial Sequence
  Description of Artificial Sequence Synthetic peptide
EILPGSGHTEYTENFKD

SEQ ID NO: 19 - protein, 10 aa, Artificial Sequence
  Description of Artificial Sequence Synthetic peptide
GHIFSNYWIQ

SEQ ID NO: 20 - protein, 448 aa, Artificial Sequence
  Description of Artificial Sequence Synthetic polypeptide
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 5 aa, Artificial Sequence
  Description of Artificial Sequence Synthetic peptide
SYAIS

SEQ ID NO: 22 - protein, 17 aa, Artificial Sequence
  Description of Artificial Sequence Synthetic peptide
GIGPFFGTANYAQKFQG

SEQ ID NO: 23 - protein, 7 aa, Artificial Sequence
  Description of Artificial Sequence Synthetic peptide
DTPYFDY

SEQ ID NO: 24 - protein, 11 aa, Artificial Sequence
  Description of Artificial Sequence Synthetic peptide
SGDSIPNYYVY

SEQ ID NO: 25 - protein, 7 aa, Artificial Sequence
  Description of Artificial Sequence Synthetic peptide
DDSNRPS

SEQ ID NO: 26 - protein, 11 aa, Artificial Sequence
  Description of Artificial Sequence Synthetic peptide
QSFDSSLNAEV

SEQ ID NO: 27 - protein, 116 aa, Artificial Sequence
  Description of Artificial Sequence Synthetic polypeptide
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 28 - protein, 108 aa, Artificial Sequence
  Description of Artificial Sequence Synthetic polypeptide
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 29 - protein, 6 aa, Artificial Sequence
  Description of Artificial Sequence Synthetic peptide
SSYYVA

SEQ ID NO: 30 - protein, 17 aa, Artificial Sequence
  Description of Artificial Sequence Synthetic peptide
AIYTGSGATYKASWAKG

SEQ ID NO: 31 - protein, 13 aa, Artificial Sequence
  Description of Artificial Sequence Synthetic peptide
DGGYDYPTHAMHY

SEQ ID NO: 32 - protein, 11 aa, Artificial Sequence
  Description of Artificial Sequence Synthetic peptide
QASQNIGSSLA

SEQ ID NO: 33 - protein, 7 aa, Artificial Sequence
  Description of Artificial Sequence Synthetic peptide
GASKTHS

SEQ ID NO: 34 - protein, 12 aa, Artificial Sequence
  Description of Artificial Sequence Synthetic peptide
QSTKVGSSYGNH

SEQ ID NO: 35 - protein, 123 aa, Artificial Sequence
  Description of Artificial Sequence Synthetic polypeptide
QVQLVESGGGLVQPGGSLRLSCAASGFTSHSSYYVAWVRQAPGKGLEWVGAIYTGSGATYKASWAKGRFTISKDTSKNQVVLTMTNMDPVDTATYYCASDGGYDYPTHAMHYWGQGTLVTVSS

SEQ ID NO: 36 - protein, 110 aa, Artificial Sequence
  Description of Artificial Sequence Synthetic polypeptide
DVVMTQSPSSLSASVGDRVTITCQASQNIGSSLAWYQQKPGQAPRLLIYGASKTHSGVPSRFSGSGSGTDFTLTISSLQPEDVATYYCQSTKVGSSYGNHFGGGTKVEIK

SEQ ID NO: 37 - protein, 451 aa, Artificial Sequence
  Description of Artificial Sequence Synthetic polypeptide
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 38 - protein, 217 aa, Artificial Sequence
  Description of Artificial Sequence Synthetic polypeptide
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 120 aa, Artificial Sequence
  Description of Artificial Sequence Synthetic polypeptide
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 40 - protein, 107 aa, Artificial Sequence
  Description of Artificial Sequence Synthetic polypeptide
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 41 - protein, 447 aa, Artificial Sequence
  Description of Artificial Sequence Synthetic polypeptide
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 214 aa, Artificial Sequence
  Description of Artificial Sequence Synthetic polypeptide
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC